(19) United States
(12) Patent Application Publication
(10) Pub. No.: US 2024/0226275 A1
(43) Pub. Date: Jul. 11, 2024

(54) COMPOSITIONS AND METHODS OF MRNA VACCINES AGAINST NOVEL CORONAVIRUS INFECTION

(71) Applicant: RNAimmune, Inc., Gaithersburg, MD (US)

(72) Inventors: Neeti Ananthaswamy, Gaithersburg, MD (US); Yong-Sik Bong, Gaithersburg, MD (US); David Brown, Gaithersburg, MD (US); Renxiang Chen, Gaithersburg, MD (US); Ju Hyeong Jeon, Gaithersburg, MD (US); Zhifeng Long, Gaithersburg, MD (US); Dong Shen, Baltimore, MD (US); Chun Lu, Montreal (CA); Patrick Y. Lu, Potomac, MD (US); Shenggao Tang, Guangzhou (CN); Jiaxi He, Guangzhou (CN); Ziyang He, Potomac, MD (US)

(73) Assignee: RNAimmune, Inc., Gaithersburg, MD (US)

(21) Appl. No.: 18/438,342

(22) Filed: Feb. 9, 2024

Related U.S. Application Data
(63) Continuation of application No. PCT/US22/39997, filed on Aug. 10, 2022.
(60) Provisional application No. 63/232,101, filed on Aug. 11, 2021.

(51) Int. Cl. A61K 39/215; A61K 9/127; A61P 37/04; C07K 14/005; A61K 39/00

(52) U.S. Cl. CPC A61K 39/215; A61K 9/1271; A61P 37/04; C07K 14/005; A61K 2039/53; C12N 2770/18022; C12N 2770/18034

(57) ABSTRACT

Provided herein is a ribonucleic acid (RNA) encoding a spike (S) protein or an immunogenic fragment thereof of a severe acute respiratory syndrome coronavirus 2 (SARS-COV-2) comprising at least one non-naturally occurring amino acid mutation. In some embodiments, the S protein is derived from a delta variant. Additionally provided are relevant polynucleotides, vectors, cells, compositions, kits, production methods and methods of use.

### CROSS-REFERENCE TO RELATED APPLICATION

This application is a continuation of International Patent Application No. PCT/US2022/039997, filed Aug. 10, 2022, which claims priority to U.S. Provisional application No. 63/232,101, filed Aug. 11, 2021, the contents of which are hereby incorporated by reference in its entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Oct. 17, 2022, is named 129774-0185.xml and is 126,559 bytes in size.

### TECHNICAL FIELD

Prophylactic and therapeutic agents for vaccination, prevention and treatment of 2019-nCOV infections are provided.

### BACKGROUND

Coronaviruses (CoVs) have repeatedly crossed species barriers and some have emerged as important human pathogens. During the past two decades, two coronaviruses infecting animals have evolved and caused outbreaks in humans: severe acute respiratory syndrome-related coronavirus (SARS-COV, 2002, genus: Betacoronavirus, subgenus: Sarbecovirus), and Middle East respiratory syndrome-related coronavirus (MERS-COV, 2012, genus: Betacoronavirus, subgenus: Merbecovirus). See, for example, Drosten et al., New Engl J Med. 2003; 348: 1967-1976; and Zaki et al., New Engl J Med. 2012; 367:1814-1820.

The SARS-COV-2 2019 (COVID-19) is a new strain of coronavirus that causes coronavirus disease. See, for example, Zhu et al., N Engl J Med. 2020, 382:727-733. Accordingly to the COVID-19 Weekly Epidemiological Update and Weekly Operational Update dated Jul. 26, 2021 and published by World Health Organization (WHO), there are close to 200 million confirmed cases and more than 4 million confirmed deaths worldwide. No cure is available for COVID-19, while steroid has been used for therapeutic management of hospitalized adults, and the Food and Drug Administration (FDA) has issued Emergency Use Authorizations (EUAs) for anti-SARS-COV-2 antibodies in treating non-hospitalized individuals with COVID-19, such as sotrovimab, and the combination of casirivimab and imdevimab. See, for example, COVID-19 Treatment Guidelines Panel. Coronavirus Disease 2019 (COVID-19) Treatment Guidelines. National Institutes of Health. available at www.covid19treatmentguidelines.nih.gov and last accessed on Jul. 27, 2021.

Accordingly, there remains an urgent need for effective prevention and treatment of a SARS-COV-2 infection. This disclosure satisfies this need and provides related advantages as well.

### SUMMARY

Provided herein are spike (s) proteins or immunologic fragments thereof as well as a deoxyribonucleic acid (DNA) or a ribonucleic acid (RNA) encoding a spike (S) protein or an immunogenic fragment thereof of a severe acute respiratory syndrome coronavirus 2 (SARS-COV-2). The S protein or immunogenic fragment thereof comprises or consists essentially of, or yet further consists of at least one non-naturally occurring amino acid mutation, for example, as compared to an S protein of a SARS-COV-2 delta variant, such as SEQ ID NO: 1 or a variant thereof. In some embodiments, the at least one non-naturally occurring amino acid mutation comprises, or alternatively consists essentially of, or yet further consists of one or more of: a serine (S) as the amino acid corresponding to R682 of SEQ ID NO: 1 (R682S), a glycine (G) as the amino acid corresponding to R685 of SEQ ID NO: 1 (R685G), a proline (P) as the amino acid corresponding to F817 of SEQ ID NO: 1 (F817P), a P as the amino acid corresponding to A892 of SEQ ID NO: 1 (A892P), a P as the amino acid corresponding to A899 of SEQ ID NO: 1 (A899P), a P as the amino acid corresponding to A942 of SEQ ID NO: 1 (A942P), a P as the amino acid corresponding to K986 of SEQ ID NO: 1 (K986P), or a P as the amino acid corresponding to V987 of SEQ ID NO: 1 (V987P). In some embodiments, the RNA encoding a spike (S) protein or an immunogenic fragment thereof does not comprise SEQ ID NO: 2. In one aspect, the RNA encoding the S protein or an immunogenic fragment thereof comprises SEQ ID NO: 55. In some embodiments, the RNA is a messenger RNA (mRNA).

In some embodiments, the mRNA encodes an S protein that further comprises, or consisting essentially of, or yet further consisting of, one or more of mutations which can be found in a SARS-COV-2 naturally occurring variant, such as a delta variant. In some embodiments, these mutations comprise, or alternatively consist essentially of, or yet further consist of one or more of: a lysine (K) as the amino acid corresponding to N440 of SEQ ID NO: 1 (N440K), a K as the amino acid corresponding to E484 of SEQ ID NO: 1 (E484K), an arginine (R) as the amino acid corresponding to T19 of SEQ ID NO: 1 (T19R), a phenylalanine (F) as the amino acid corresponding to V70 of SEQ ID NO: 1 (V70F), an isoleucine (I) as the amino acid corresponding to T95 of SEQ ID NO: 1 (T95I), an aspartic acid (D) as the amino acid corresponding to G142 of SEQ ID NO: 1 (G142D), a deletion corresponding to E156 of SEQ ID NO: 1 (E156Δ), a deletion corresponding to F157 of SEQ ID NO: 1 (F157Δ), a G as the amino acid corresponding to R158 of SEQ ID NO: 1 (R158G), a valine (V) as the amino acid corresponding to A222 of SEQ ID NO: 1 (A222V), a leucine (L) as the amino acid corresponding to W258 of SEQ ID NO: 1 (W258L), an asparagine (N) as the amino acid corresponding to K417 of SEQ ID NO: 1 (K417N), an R as the amino acid corresponding to K417 of SEQ ID NO: 1 (L452R), a K as the amino acid corresponding to T478 of SEQ ID NO: 1 (T478K), a G as the amino acid corresponding to D614 of SEQ ID NO: 1 (D614G), an R as the amino acid corresponding to P681 of SEQ ID NO: 1 (P681R), or an N as the amino acid corresponding to D950 of SEQ ID NO: 1 (D950N).

In some embodiments, the S protein or an immunogenic fragment thereof comprises, or alternatively consists essentially of, or yet further consists of the polypeptide of any one of SEQ ID NO: 5, 6, 7, 10, 11, or 14, or an equivalent of each thereof. This disclosure further provides the RNA or DNA encoding these polypeptides and equivalents thereof. In some embodiments, the RNA comprises, or alternatively consists essentially of, or yet further consists of the polynucleotide of SEQ ID NO: 9, 13, or 16, or an equivalent of each thereof. In some embodiments, the RNA further comprises one or more of a three prime untranslated region (3′ UTR), a polyadenylation (polyA) tail and a five prime untranslated region (5′ UTR). In some embodiments, the RNA comprises, or alternatively consists essentially of, or yet further consists of SEQ ID NO: 32. SEQ ID NOs: 9, 13, 16 encode S polypeptides. SEQ ID NO: 32 is SEQ ID NO: 9, further comprising the 5 and 3′ UTRs and a polyA tail.

In one aspect, provided is a polynucleotide, such as a DNA, encoding an RNA as disclosed herein. Also provided are complements of the encoding DNA. In a further aspect, provided is a vector comprising a polynucleotide as disclosed herein. In one embodiment, the vector is a plasmid, optionally comprising, or alternatively consisting essentially of, or yet further consisting of SEQ ID NO: 33 or an equivalent thereof. In yet a further aspect, provided is a cell comprising one or more of: an RNA as disclosed herein, a polynucleotide as disclosed herein, or a vector as disclosed herein. In one aspect, provided is a composition comprising, or alternatively consisting essentially of, or yet further consisting of a carrier, optionally a pharmaceutically acceptable carrier and one or more of: an RNA as disclosed herein, a polynucleotide as disclosed herein, a vector as disclosed herein, or a cell as disclosed herein.

Additionally provided is a method of producing DNA or an RNA as disclosed herein. In some embodiments, the method comprises, or alternatively consists essentially of, or yet further consisting of culturing a cell as disclosed herein under conditions suitable for expressing the DNA and/or the RNA. In other embodiments, the method comprises, or alternatively consists essentially of, or yet further consists of contacting a polynucleotide or a vector as disclosed herein with an RNA polymerase, adenosine triphosphate (ATP), cytidine triphosphate (CTP), guanosine-5′-triphosphate (GTP), and uridine triphosphate (UTP) or a chemically modified UTP (such as N1-methyl pseudouridine trisphosphate) under conditions suitable for expressing the RNA. In further embodiments, a method as disclosed herein further comprises isolating the DNA or the RNA.

In one aspect, provided is a composition comprising, or alternatively consisting essentially of, or yet further consisting of a protein, a polypeptide, an RNA or DNA as disclosed herein and a carrier such as a pharmaceutically acceptable carrier. In some embodiments, the pharmaceutically acceptable carrier comprises, or alternatively consists essentially of, or yet further consists of a polymeric nanoparticle. In further embodiments, the polymeric nanoparticle comprises, or alternatively consists essentially of, or yet further consists of a Histidine-Lysine co-polymer (HKP). In some embodiments, the pharmaceutically acceptable carrier further comprises a lipid, optionally one or more of: a cationic lipid (such as Dlin-MC3-DMA, i.e., MC3), a helper lipid, a cholesterol, or a PEGylated lipid. In some embodiments, the pharmaceutically acceptable carrier comprises, or alternatively consists essentially of, or yet further consists of a lipid nanoparticle (LNP). In some embodiments, the LNP comprises one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof. In further embodiments, the LNP further comprises one or more of: a helper lipid, a cholesterol, or a PEGylated lipid. A further pharmaceutically acceptable carrier can be added to the nanoparticle composition, e.g., phosphate buffered saline and the like.

In further embodiments, the pharmaceutically acceptable carrier further comprises a dilute, an adjuvant, a binder, a stabilizer, a buffer, a salt, a lipophilic solvent, or a preservative. In some embodiments, the nanoparticle is a self-assembled nanoparticle. In a further aspect, provided is a composition comprising, or alternatively consisting essentially of, or yet further consisting of a self-assembled nanoparticle comprising an RNA as disclosed herein. In some embodiments, the nanoparticle encapsulates the RNA. In other embodiments, the nanoparticle is conveniently or non-covalently linked to the RNA.

In a further embodiments, provided is a method of producing the composition. In some embodiments, the method comprises, or alternatively consists essentially of, or yet further consists of contacting an RNA as disclosed herein with an HKP, thereby the RNA and the HKP are self-assembled into nanoparticles. Additionally or alternatively, the method comprises, or alternatively consists essentially of, or yet further consist of contacting an RNA as disclosed herein with a lipid, thereby the RNA and the lipid are self-assembled into nanoparticles. In further embodiment, the contacting step is performed in a microfluidic mixer, such as NanoAssemblr Ignite.

In another aspect, provided is a method of one or more of: (a) preventing a subject from having a symptomatic SARS-COV-2 infection, (b) inducing an immune response to SARS-COV-2 in a subject in need thereof, (c) reducing the binding of a SARS-COV-2 or an S protein thereof with angiotensin converting enzyme 2 (ACE2) in a subject in need thereof, (d) treating a subject infected with SARS-COV-2, or (c) reducing a SARS-COV-2 viral load in a subject in need thereof. The method comprises, or alternatively consists essentially of, or yet further consists of administering to the subject one or more of: an RNA or DNA as disclosed herein, a polynucleotide as disclosed herein, a vector as disclosed herein, or a composition as disclosed herein.

In one aspect, provided is an inhalation system comprising, or alternatively consisting essentially of, or yet further consisting of an RNA or DNA as disclosed herein, a polynucleotide as disclosed herein, a vector as disclosed herein, or a composition as disclosed herein, and a nebulizer.

In another aspect, provided is a method of producing a spike (S) protein or an immunogenic fragment thereof of a severe acute respiratory syndrome coronavirus 2 (SARS-CoV-2). The method comprises, or alternatively consists essentially of, or yet further consists of culturing a cell as disclosed herein under conditions suitable for expressing the S protein or immunogenic fragment thereof. In further embodiments, the method herein further comprises isolating the S protein or immunogenic fragment thereof. Alternatively or in addition, the S protein or an immunogenic fragment thereof can be produced by administering a composition comprising the DNA and/or RNA to a subject.

Additionally, provided is a method for screening a candidate agent reducing or inhibiting the binding of SARS-COV-2 and its receptor, such as ACE2, optionally in a subject or on a cell of the subject or both. The method comprises, or alternatively consist essentially of, or yet further consists of expressing a spike (S) protein or an immunogenic fragment thereof from an RNA as disclosed herein, and measuring the binding between the expressed S protein or immunogenic fragment thereof and the SARS-COV-2 receptor, such as ACE2, with or without the presence of the candidate agent or with different concentrations of the candidate agent. In some embodiments, less binding between the expressed S protein or immunogenic fragment thereof and the SARS-COV-2 receptor with the presence of the candidate agent compared to without the candidate agent, indicates that the candidate agent reduces or inhibits the binding of SARS-COV-2 and its receptor. In some embodiments, decreased binding between the expressed S protein or immunogenic fragment thereof and the SARS-COV-2 receptor while increasing the concentration of the candidate agent indicates the candidate agent reduces or inhibits the binding of SARS-COV-2 and its receptor.

In yet a further aspect, provided is a method for selecting an RNA encoding a spike (S) protein or an immunogenic fragment thereof of a severe acute respiratory syndrome coronavirus 2 (SARS-COV-2). The method comprises, or alternatively consists essentially of, or yet further consists of transducing the RNA into a cell, culturing the cell under conditions suitable for expressing the RNA, and measuring IFN-α or IFN-β or both secreted by the cell. In some embodiments, the method further comprises selecting the RNA if no secretion of IFN-α or IFN-β or both or less secretion of IFN-α or IFN-β or both compared to an RNA encoding a wild type S protein or an immunogenic fragment thereof.

Also provided is a kit for use in a method as described herein. In some embodiments, the kit comprises, or alternatively consists essentially of, or yet further consists of instructions for use and one or more of: an RNA as disclosed herein, a polynucleotide as disclosed herein, a vector as disclosed herein, a composition as disclosed herein, or an inhalation system as disclosed herein.

### BRIEF DESCRIPTION OF THE DRAWINGS

FIG. 1 provides a representative parameter setting of NanoAssemblr Ignite for producing an RNA in a RL-007 lipid. RL-007 is prepared by mixing a final concentration of 6.25 mM of SM-102, 1.25 mM of DSPC, 4.815 mM of Cholesterol, and 0.1875 of mM DMG-PEG2000 (i.e., a 50:10:38:1.5 molar ratio). 0.13 mg/mL of mRNA was mixed in a 3:1 (v/v) ratio with RL-007.

FIGS. 2A-2C provide representative images showing expression of a Blue Fluorescent Protein in cells transfected with RNAs encoding the Blue Fluorescent Protein and comprising various polyA tails (FIG. 2A, polyA 40 (SEQ ID NO: 27 or 56); and FIG. 2B, polyA 60 (SEQ ID NO: 28 or 57)) or no PolyA tail (FIG. 2C).

FIG. 3 provides a bar graph showing expression of a Blue Fluorescent Protein indicated by fluorescence intensity measured at various time points (0 hour, 24 hours, 48 hours, 72 hours, 96 hours and 144 hours) post transfection of RNAs encoding the Blue Fluorescent Protein and comprising different UTRs. The first set of bars from the left represents data acquired using β-globulin 5′ UTR. The second set of bars from the left represents data acquired using SYS 5′ UTR as disclosed herein. The third set of bars from the left represents data acquired using no 5′ UTR. The fourth set of bars from the left represents data acquired from the blank control group. Error bars indicate the standard deviation of 3 replicates.

FIG. 4 shows representative IFN-α and IFN-β ELISA results as detailed in Example 9. S WT stands for spike protein wild type. S RBD stands for spike receptor binding protein. S2P stands for comprising the K986P and V987P mutations. S6P stands for comprising the mutations of F817P, A892P, A899P, A942P, K986P, and V987P. UK stands for the alpha variant firstly identified in United Kingdom. SA stands for the beta variant firstly identified in South Africa. GS stands for a sequence-optimized RNA (SEQ ID NO: 54). A549 indicates negative control A549 lung carcinoma epithelial cells.

FIGS. 5A-5B provide a quantitative result of an IFN-α ELISA as demonstrated in Example 9. FIG. 5A is the standard curve while FIG. 5B provides the corresponding calculation.

FIGS. 6A-6B provide a quantitative result of an IFN-β ELISA result as demonstrated in Example 9. FIG. 6A is the standard curve while FIG. 6B provides the corresponding calculation.

FIG. 7 shows a clinical severity score curve in immunized and non-immunized mice after challenge with SARS-COV-2/human/ITA/INMI1/2020 virus. Mice in Group 1 were administered PBS, Group 2 mice were immunized with mRNA RL-007 1 μg/animal, Group 3 mice were immunized with mRNA RL007 5 μg/animal, Group 4 mice were immunized with mRNA RL007 10 μg/animal before viral challenge.

FIGS. 8A-8D show body weights of treated mice. FIG. 8A shows group mean body weights of non-immunized and immunized mice. FIG. 8B shows group mean body weights of non-immunized and immunized mice after viral challenge with SARS-COV-2/human/ITA/INMI1/2020 isolate (NCBI Accession number: MT066156). Body weight in mice of Groups 1 to 4 after immunization at NLS, and after viral challenge with SARS-COV-2/human/ITA/INMI1/2020 isolate. Body weight of each individual mouse was calculated as a percentage of body weight measured after delivery to NLS, and as a percentage of body weight measured before viral challenging at GMU. The mean and standard error of the mean (SEM) for each group of mice are presented. Group 1—“C” is represents control non-immunized mice. FIG. 8C shows group mean body weights of non-immunized and immunized mice prior to viral challenge with SARS-COV-2 B.1.1617.2 Delta variant. FIG. 8D shows group mean body weights of non-immunized and immunized mice after viral challenge with SARS-COV-2 B.1.1617.2 Delta variant. m-Co is a control vaccine delivered at Low Dose (0.50 μg/mouse) and High Dose (5 μg/mouse). Non-control mice were vaccinated with RV-1730 (labeled “RNAImmune in FIG. 8C and FIG. 8D) which uses an RL-007 carrier.

FIG. 9 shows body temperature curves in mice of Groups 1 to 4 after viral challenge with SARS-COV-2/human/ITA/INMI1/2020 (NCBI Accession number: MT066156). The mean and standard error of the mean (SEM) for each group of mice are represented. Mice in Group 1 were administered PBS, Group 2 mice were immunized with mRNA RL-007 1 μg/animal, Group 3 mice were immunized with mRNA RL007 5 μg/animal, Group 4 mice were immunized with mRNA RL007 10 μg/animal before viral challenge. Experiments are described in Example 10.

FIG. 10 is a Kaplan Meier curve showing the survival rate for mice challenged with SARS-COV-2. Mice in Group 1 were administered PBS, Group 2 mice were immunized with mRNA RL-007 1 μg/animal, Group 3 mice were immunized with mRNA RL007 5 μg/animal, Group 4 mice were immunized with mRNA RL007 10 μg/animal before viral challenge. Experiments are described in Example 10.

FIG. 11A, FIG. 11B, FIG. 11C, and FIG. 11D show mouse serum titration data against SARS-COV-2/human/ITA/INMI1/2020 assessed using plaque reduction neutralization titration (PRNT). The neutralizing titers were calculated as the reciprocal of the lowest dilution that resulted in a greater than 50% reduction (PRNT₅₀) or 90% reduction (PRNT₉₀) in plaque forming units (p.f.u.) relative to negative control sera. FIG. 11A shows PRNT₅₀ of post-prime immunization mouse serum collected on Day 13; FIG. 11B shows PRNT₉₀ of post-prime immunization mouse serum collected on Day 13; FIG. 11C shows PRNT₅₀ of post-boost immunization mouse serum collected on Day 27; FIG. 11D shows PRNT₉₀ of post-boost immunization mouse serum collected on Day 27. Symbols and horizontal lines represent individual titers of each sample and mean titers of each group, respectively. Serum titers were expressed as reciprocals Log₂ dilution. Experiments are described in Example 10.

FIG. 12 shows histopathology score for lung tissues harvested at euthanasia. The histopathology score is graphical representation of the Total lung score, Interstitial pneumonia (pn), and Pervascular cuffs scored on a scale based on percentage of lung involved (0=none; 1=0-25%; 2=26-50%; 3=51-75%; and 4=76-100%) and (specific lung lesions: 0=none; 1=minimal; 2=mild; 3=moderate; and 4=severe) for each group. A portion of lung tissue for mice designated for euthanasia on Day 3 post SARS-COV-2/human/ITA/INMI1/2020 challenge was analyzed for viral titers in lung tissues. Viral titers in lung tissues were determined in five mice (5) from each group three (3) days post viral challenge. Group 1 mice were administered PBS control (identified as “C” in FIG. 7). Groups 2, 3, and 4 mice were immunized with and RL007 mRNA vaccine with dose 1 μg/animal, 5 μg/animal, 10 μg/animal, respectively. Viral titers were calculated as p.f.u./mL. Experiments are described in Example 11

FIG. 13 shows histopathology images for representative lung tissues of mice treated as disclosed in FIG. 7, harvested 13 days post-challenge with SARS-COV-2. H&E stained tissue slides from representative mice for each group were scanned at 20× magnification. Group 1 mice were administered PBS control. Groups 2, 3, and 4 mice were immunized with mRNA vaccine RL007 with dose 1 μg/animal, 5 μg/animal, 10 μg/animal, respectively. Top left: Group 1, mouse R24-101-010; Top Right: Group 2, mouse R24-101-013; Bottom Left: Group 3, mouse R24-101-023; Bottom Right: Group 4, mouse R24-101-034. Experiments are described in Example 11.

FIG. 14 shows Th-1 and Th-2 cytokine measurements of mice treated as disclosed in FIG. 7 nine weeks post-boost. Splenocytes were isolated from 5 mice per group and re-stimulated with no peptides or pools of overlapping peptides from SARS-COV-2 S protein. After 72 hours, the culture supernatants were harvested by centrifugation and the secreted Th1-cytokines (IFN-γ, IL-2, TNF-α) and Th2-cytokines (IL-4, IL-5, IL-13) were measured using the bead-based, 11-plex TH1/TH2 mouse ProcartaPlex multiplex immunoassay (Thermo Fisher Scientific). Fluorescence was measured with a Luminex 100/200 system and analyzed with ProcartaPlex Analyst 1.0 software (Thermo Fisher Scientific). Below the lower limit of quantification were set to zero. **P<0.01, ***P<0.001, and ****P<0.0001 (unpaired Student's t-test, compared to PBS control). Experiments are described in Example 14.

FIG. 15A, FIG. 15B, and FIG. 15C show IFN-γ ELISPOT analysis on splenocytes. 9 weeks post-boost, splenocytes were isolated from 5 mice per group, then splenocytes (2×10⁵ cells per 96 well) were plated onto mouse IFN-γ ELISpot plates (Mabtech) and re-stimulated ex vivo with pools of overlapping peptides from SARS-COV-2 Delta Spike protein subunits S1 and S2 for 16 hours. FIG. 15A shows images taken and quantified by using Cytation7. *P<0.05, ****P<0.001. FIG. 15B is a graph quantifying IFN-γ spots per 2×10⁵ cells re-stimulated ex vivo with Delta Spike protein subunit S1. FIG. 15C is a graph quantifying IFN-γ spots per 2×10⁵ cells re-stimulated ex vivo with Delta Spike protein subunit S2. Experiments are described in Example 15.

FIG. 16 shows cytokine analysis of memory T cells using a flow cytometry. Nine weeks post-boost, splenocytes were isolated from 5 mice per group and re-stimulated with no peptides or pools of overlapping peptides from SARS-COV-2 S protein in the presence of a protein transport inhibitor cocktail. Mice were treated as demonstrated in Example 16. After 16 hours, intracellular cytokine staining (ICS) was performed to quantify CD4⁺ and CD8⁺ T cell responses. Cytokine expression in the presence of no peptides was considered background and subtracted from the responses measured from the S1 and S2 peptide pools for each individual mouse. Experiments are described in Example 16.

FIG. 17 shows endpoint IgG titers of RV-1730 immunized sera against different variant spike proteins. RV-1730 is a representative RL-007 mRNA vaccine formulation, prepared according to Example 3, comprising SEQ ID NO: 52. SEQ ID NO: 52 comprises the open reading frame depicted in SEQ ID NO: 55. RV-1730 uses a RL-007 carrier prepared according to Example 3. Experiments are described in Example 12.

FIG. 18A and FIG. 18B show neutralization antibody titers against SARS-CoV-2 Pseudovirus particles (PP) after RV-1730 immunization at day 14 (FIG. 18A) and day 35 (FIG. 18B) as demonstrated in Example 13. Data are shown for Brazil: Gamma variant, 614D: wild-type, UK: Alpha variant, Delta: Delta variant, 614G: D614G variant, SA: Beta variant pseudovirus particles. Experiments are described in Example 13.

FIGS. 19A-19D show the effect of immunized mouse sera on different SARS-COV-2-PPs infection across five groups of mice (G1, G2, G3, G4 and G5). The x-axis shows compound concentration and the Y-axis shows relative PP infectivity. Data are shown for 614D-PP (FIG. 19A), Delta-PP (FIG. 19B), Omicron-PP (FIG. 19C), BA.2-PP (FIG. 19D). Experiments are described in Example 13.

FIG. 20 is a graph of IC₅₀ (serum %) of heterologous vaccination sera in a pseudovirus neutralizing assay across five groups of mice (G1, G2, G3, G4 and G5). Neutralization antibody titers against SARS-COV-2 pseudovirus particles including wild-type SARS-COV-2, Delta SARS-COV-2, Omicron BA.1 SARS-COV-2, and Omicron BA.2 SARS-CoV-2, were measured following first and second SARS-COV-2 mRNA vaccine immunization doses. Mice received a combination of first and second vaccine immunization doses of RV-1730, or a SARS-COV-2 vaccine as known in the art, such as BNT162b2 available from Pfizer-BioNTech, or mRNA-1273 available from Moderna. Experiments are described in Example 13.

### DETAILED DESCRIPTION

### Definitions

As it would be understood, the section or subsection headings as used herein is for organizational purposes only and are not to be construed as limiting and/or separating the subject matter described.

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press); and Plotkin et al., Plotkin; s Human Vaccines, 7^{th} edition (Elsevier).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

In some embodiments, the terms “first” “second” “third” “fourth” or similar in a component name are used to distinguish and identify more than one components sharing certain identity in their names. For example, “first RNA” and “second RNA” are used to distinguishing two RNAs.

Severe acute respiratory syndrome coronavirus 2 (SARS-COV-2), also referred to as 2019 novel coronavirus (2019-nCOV) or human coronavirus 2019 (HCoV-19 or hCoV-19), is the virus that causes COVID-19 (coronavirus disease 2019), the respiratory illness responsible for the COVID-19 pandemic.

Each SARS-COV-2 virion is 50-200 nanometers in diameter, comprising a linear, positive-sense, single-stranded RNA genome (about 30,000 bases long) and four structural proteins, known as the S (spike), E (envelope), M (membrane), and N (nucleocapsid) proteins. The N protein holds the RNA genome, and the S, E, and M proteins together create the viral envelope. Coronavirus S proteins are glycoproteins that are divided into two functional parts (S1 and S2). In SARS-COV-2, the spike protein is the protein responsible for allowing the virus to attach to and fuse with the membrane of a host cell; specifically, its S1 subunit catalyzes attachment, the S2 subunit fusion. Studies have shown that SARS-COV-2 has sufficient affinity to the receptor angiotensin converting enzyme 2 (ACE2) on human cells to use them as a mechanism of cell entry. Initial spike protein priming by transmembrane protease, serine 2 (TMPRSS2) is also shown as essential for entry of SARS-COV-2. The host protein neuropilin 1 (NRP1) may aid the virus in host cell entry using ACE2. After a SARS-COV-2 virion attaches to a target cell, the cell's TMPRSS2 cuts open the spike protein of the virus, exposing a fusion peptide in the S2 subunit, and the host receptor ACE2. After fusion, an endosome forms around the virion, separating it from the rest of the host cell. The virion escapes when the pH of the endosome drops or when cathepsin, a host cysteine protease, cleaves it. The virion then releases RNA into the cell and forces the cell to produce and disseminate copies of the virus, which infect more cells.

Genetic variants of SARS-COV-2 have been emerging and circulating around the world throughout the COVID-19 pandemic. The B.1.1.7 (Alpha), B.1.351 (Beta), B.1.617.2 (Delta), and P.1 (Gamma) variants circulating in the United States are classified as variants of concern. Other variants are also present, such as B.1.526 (Iota), B.1.427 (Epsilon), B.1.429 (Epsilon), B.1.617 (Kappa, Delta), B.1.525 (Eta), and P.2 (Zeta). Accordingly, the term “SARS-COV-2” as used herein can refer to any one or more or all of the variants. In some embodiments, SARS-COV-2 as used herein refers to a delta variant, which was first identified in India. In further embodiments, SARS-COV-2 delta variant comprises mutations in the gene encoding the S protein causing one or more of the following amino acid mutations in the S protein: T19R, V70F, T95I, G142D, E156Δ, F157Δ, R158G, A222V, W258L, K417N, L452R, T478K, D614G, P681R, or D950N. In some embodiments, the delta variant is a delta plus variant comprising the amino acid mutation of K417N.

Subjects with a SARS-COV-2 infection can experience a range of clinical manifestations, from an asymptomatic SARS-COV-2 infection with no symptoms, to a symptomatic SARS-COV-2 infection with critical illness. SARS-COV-2 infection can be grouped into the following severity of illness categories; however, the criteria for each category may overlap or vary across clinical guidelines and clinical trials, and a patient's clinical status may change over time. Asymptomatic or presymptomatic SARS-CoV-2 infection occurs when a subject tests positive for SARS-COV-2 using a virologic test, but exhibits no symptoms (e.g., fever, cough, sore throat, malaise, headache, muscle pain, nausea, vomiting, diarrhea, loss of taste and smell) consistent with a symptomatic SARS-COV-2 infection. A symptomatic SARS-COV-2 infection occurs when a subject exhibits mild, moderate, severe, or critical illness associated with SARS-COV-2 infection, as defined by the U.S. National Institutes of Health. Subjects with mild illness associated with SARS-COV-2 infection exhibit symptoms including, but not limited to, fever, cough, sore throat, malaise, headache, muscle pain, nausea, vomiting, diarrhea, loss of taste and smell, but do not have shortness of breath, dyspnea, or abnormal chest imaging. Subjects with moderate illness associated with SARS-COV-2 infection show evidence of lower respiratory disease during clinical assessment or imaging and have an oxygen saturation (SpO₂)≥94% on room air at sea level. Subjects with severe illness associated with SARS-COV-2 infection have SpO₂<94% on room air at sea level, a ratio of arterial partial pressure of oxygen to fraction of inspired oxygen (PaO₂/FiO₂)<300 mm Hg, a respiratory rate >30 breaths/min, or lung infiltrates >50%. Subjects with critical illness associated with SARS-COV-2 infection have respiratory failure, septic shock, and/or multiple organ dysfunction.

Symptoms of a coronavirus infection include, but are not limited to, mild symptoms, such as fatigues, tingling, tingling or numbness in the hands and feet, dizziness, confusion, brain fog, body ache, chills, loss of appetite, nausea, vomiting, abdominal pain or discomfort, loss of smell, inability to taste, muscle weakness, photophobia, adenopathy, headaches, cough, dry cough, shortness of breath, sore throat, lower extremity weakness/numbness, diarrhea, low blood O₂, sneezing, runny nose or post-nasal drip; severe symptoms, such as ventilatory use, high fever, severe cough, delirium, seizures, stroke, systematic inflammation, cytokine storm; and other symptoms, such as fever, swollen adenoids, pneumonia, bronchitis, and Dyspnea.

Viral infection of a coronavirus, such as SARS-COV-2, can be detected via a commercially available test known in the art, for example via polymerase chain reaction (PCR) or immunoassay may be used. In some embodiments, a method as disclosed herein further comprises detecting a coronavirus using a test known in the art. In one embodiment, active viral infection refers to an ongoing infection wherein the virus is replicating and producing new virus. Such active viral infection may be detected using polymerase chain reaction (PCR). Non-limiting examples of primers and probes suitable for use in the PCR include 2019-nCOV CDC Probe and Primer Kit for SARS-COV-2 (BioSearch Technologies, Catalog No. KIT-nCOV-PP1-1000), 2019-nCOV Kit, 500 rxn (Integrated DNA Technologies (IDT), Catalog No. 10006606) and 2019-nCOV Kit, 1000 rxn (Integrated DNA Technologies (IDT), Catalog No. 10006770). Also see, www.cdc.gov/coronavirus/2019-ncov/lab/rt-pcr-panel-primer-probes.html and www.cdc.gov/coronavirus/2019-ncov/downloads/List-of-Acceptable-Commercial-Primers-Probes.pdf. Suitable protocols for performing such tests can be found at www.cdc.gov/coronavirus/2019-ncov/lab/virus-requests.html, www.fda.gov/media/134922/download, www.cdc.gov/coronavirus/2019-ncov/downloads/processing-sputum-specimens.pdf, www.fda.gov/media/134922/download, www.fda.gov/media/134919/download, www.fda.gov/media/134922/download, last accessed on Aug. 10, 2021. In some embodiments, diagnostic assays for COVID-19 based on detecting antibodies is combined with those disclosed herein, such as those discussed by Lisboa Bastos M et al. (Diagnostic accuracy of serological tests for covid-19: systematic review and meta-analysis. BMJ. 2020 Jul. 1; 370:m2516. doi: 10.1136/bmj.m2516).

Other commercially available tests include, but not limited to those listed in the Table below.

Commercially available tests for SARS-CoV-2 and COVID-19

| Company Name | Test Name |
|---|---|
| 3D Medicines | SARS-CoV-2 and Influenza A & B RT-qPCR |
| | Detection Kit |
| Abbott | SARS-CoV-2 IgG test |
| Abbott | ID Now COVID-19 |
| Abbott | Abbott RealTime SARS-CoV-2 EUA test |
| Anatolia Geneworks | Bosphore Novel Coronavirus (2019-nCoV) |
| | Detection Kit |
| ARUP Laboratories | COVID-19 |
| A*STAR, Tan Tock Seng | A*STAR Fortitude 2.0 |
| Hospital of Singapore | |
| Assure Tech | COVID-19 IgG/IgM Rapid Test Device |
| Atila BioSystems | iAMP COVID-19 Detection Kit |
| AusDiagnostics | AusDiagnostics SARS-CoV-2, influenza, RSV panel |
| Autobio Diagnostics | Anti-SARS-CoV-2 Rapid Test |
| Avellino Lab | Avellino SARS-CoV-2/COVID-19 (AvellinoCoV2) |
| Bako Diagnostics | BakoDx SARS-CoV-2 RNA test |
| Baptist Hospital Miami | COVID-19 RT-PCR Test |
| Pathology/Laboratory | |
| Medicine Lab | |
| Becton Dickinson | BD SARS-CoV-2 Reagents for BD MAX System |
| Becton Dickinson, BioGx | BioGX SARS-CoV-2 Reagents for the BD MAX |
| | System |
| Beijing Decombio | Novel Coronavirus IgM/IgG Combo Rapid Test- |
| Biotechnology | Cassette |
| Beijing Diagreat | 2019-nCoV IgG, IgM Antibody Determination Kits |
| Biotechnologies | 2019-nCoV IgG/IgM Antibody Rapid Test Kit |
| Beijing Kewei Clinical | Genonto RapidTest10 COVID-19 IgG/IgM Antibody |
| Diagnostic Reagent | Rapid Test Kit |
| Beijing O&D Biotech | Coronavirus disease (COVID-19) Total Antibody |
| | Rapid Test (Colloidal Gold) |
| Beroni Group | SARS-CoV-2 IgG/IgM Antibody Detection Kit |
| BGI | Real-Time Fluorescent RT-PCR kit for detecting |
| | SARS-2019-nCoV |
| Biodesix | SARS-CoV-2 Droplet Digital PCR (ddPCR) test |
| Biolidics | 2019-nCoV IgG/IgM Detection Kit (Colloidal Gold) |
| BioMedomics | COVID-19 IgM-IgG Rapid Test |
| BioMérieux | SARS-COV-2 R-GENE test |
| BioMérieux/BioFire Defense | BioFire COVID-19 test |
| Bioneer | AccuPower COVID-19 Real-Time RT-PCR Kit, |
| | AccuPower SARS-CoV-2 Real-Time RT-PCR Kit |
| Bio-Rad Laboratories | SARS-CoV-2 Total Ab test |
| BioReference Laboratories | Novel Coronavirus COVID-19 |
| Boston Children's Hospital | Childrens-Altona-SARS-CoV-2 assay |
| Infectious Diseases Diagnostic | |
| Laboratory (IDDL) | |
| BTNX | Rapid Response COVID-19 IgG/IgM Test Cassette |
| Cellex | qSARS-CoV-2 IgG/IgM Rapid Test |
| Centers for Disease Control and | CDC 2019-Novel Coronavirus (2019-nCoV) Real- |
| Prevention (performed at | Time RT-PCR Diagnostic Panel (CDC) |
| qualified high-complexity CLIA | |
| laboratories designated by CDC) | |
| Cepheid | Xpert Xpress SARS-CoV-2 test |
| CerTest BioTec | ViaSure SARS-CoV-2 Real Time PCR Detection Kit |
| Chembio Diagnostics | DDP COVID-19 IgM/IgG System |
| Children's Hospital of | SARS-CoV-2 RT-PCR test |
| Philadelphia Infectious Disease | |
| Diagnostics Laboratory | |
| ChromaCode | HDPCR SARS-CoV-2 real-time PCR assay |
| CirrusDx Laboratories | CirrusDx SARS-CoV-2 Assay |
| Co-Diagnostics | Logix Smart Coronavirus Disease 2019 (COVID-19) |
| | Kit |
| Core Technology | CoreTest COVID-19 IgM/IgG Ab Test |
| Credo Diagnostics Biomedical | VitaPCR SARS-CoV2 Assay |
| DiaCarta | QuantiVirus SARS-CoV-2 test kit |
| Diagnostic Solutions Laboratory | COVID-19 Assay |
| DiaSorin Molecular | Simplexa COVID-19 Direct |
| Diatherix Eurofins | COVID-19 Panel |
| Diazyme Laboratories | Diazyme DZ-LITE SARS-CoV-2 IgG, IgM CLIA |
| | Kits |
| Eachy Biopharmaceuticals | AccuRapid SARS-CoV-2 IgM/IgG Test Kit (Lateral |
| | Flow Immunoassay) |
| Euroimmun/PerkinElmer | EuroRealTime SARS-CoV-2 |
| Euroimmun/PerkinElmer | Anti-SARS-CoV-2 ELISAs (IgA and IgG) |
| Exact Sciences | SARS-CoV-2 Test |
| Fosun Pharma USA | Fosun COVID-19 RT-PCR Detection Kit |
| Fulgent Genetics/MedScan | COVID-19 |
| Laboratory | |
| Genetic Signatures | EasyScreen SARS-CoV-2 detection kit |
| Genetron | Detection Kit for Novel Coronavirus (SARS-CoV-2) |
| | RNA (PCR-Fluorescence Probing) |
| GenMark Diagnostics | ePlex SARS-CoV-2 Test |
| Genomica/PharmMar Group | 2 kits: qCOVID-19, CLART COVID-19 |
| GenoSensor | GS COVID-19 RT-PCR Kit |
| Gnomegen | Gnomegen COVID-19 RT-Digital PCR Detection Kit |
| Gold Standard Diagnostics | SARS-CoV-2 IgG, IgM, IgA assays |
| Guangzhou Wondfo Biotech | SARS-CoV-2 Antibody Test |
| Hackensack University Medical | CDI Enhanced COVID-19 Test |
| Center (HUMC) Molecular | |
| Pathology Laboratory | |
| Hangzhou AllTest Biotech | AllTest 2019-nCoV IgG/IgM Rapid Test Cassette, |
| | AllTest COVID IgG/IgM Rapid Test Dipstick |
| Hangzhou Biotest Biotech | COVID-19 IgG/IgM Rapid Test Cassette |
| Hangzhou Clongene Biotech | Clungene COVID-19 IgM/IgG Rapid Test Cassette |
| Hangzhou Testsealabs | One Step SARS-CoV2 (COVID-19) IgG/IgM Test |
| Biotechnology | |
| Healgen Scientific | COVID-19 IgG/IgM Rapid Test Cassette(Whole |
| | Blood/Serum/Plasma) |
| Hologic | Panther Fusion SARS-CoV-2 assay |
| InBios International | Smart Detect SARS-CoV-2 rRT-PCR Kit |
| Innovita (Tangshan) Biological | 2019-nCoV Ab Test (Colloidal Gold) |
| Technology | |
| Integrated DNA | IDT 2019-novel coronavirus kit |
| Technologies/Danaher | |
| Integrity Laboratories | SARS-CoV-2 Assay |
| Ipsum Diagnostics | COV-19 IDx assay |
| Jiangsu Macro & Micro-Test | SARS-CoV-2 IgM/IgG Rapid Assay Kit (Colloidal |
| Med-Tech | Gold) |
| JN Medsys | ProTect Covid-19 kit |
| Kogene Biotech | 2019 Novel Coronavirus Real-time PCR Kit |
| KorvaLabs | Curative-Korva SARS-Cov-2 Assay |
| Laboratory Corporation of | LabCorp 2019 Novel Coronavirus test |
| America | |
| LGC, Biosearch Technologies | 2019-nCoV CDC-qualified Probe and Primer Kits for |
| | SARS-CoV-2 |
| Lifeassay Diagnostics | Test-it COVID-19 IgM/IgG Lateral Flow Assay |
| Luminex | ARIES SARS-CoV-2 Assay |
| Luminex | NxTAGCoV Extended Panel Assay |
| Maccura Biotechnology | SARS-CoV-2 Fluorescent PCR Kit |
| Massachusetts General Hospital | MGH COVID-19 qPCR assay |
| Medical Systems Biotechnology | Coronavirus Disease 2019 Antibody (IgM/IgG) |
| | Combined Test Kit |
| Mesa Biotech | Accula SARS-CoV-2 test |
| Mount Sinai Labs | COVID-19 ELISA IgG Antibody Test |
| Nanjing Liming Bio-products | SARS-CoV-2 IgM/IgG Antibody Rapid Test Kit |
| NanoResearch | NanoMedicina SARS-CoV-2 IgM/IgG Antibody |
| | Rapid Test |
| Nantong Diagnos | (2019-nCoV) New coronavirus Antibody Test |
| Biotechnology | (Colloidal Gold) |
| NeuMoDx Molecular | NeuMoDx SARS-CoV-2 Assay |
| Nirmidas Biotech | COVID-19 (SARS-CoV-2) IgM/IgG Antibody |
| | Detection Kit |
| Northwestern Medicine | SARS-Cov-2 Assay |
| Diagnostic Molecular Laboratory | |
| Novacyt/Primerdesign | COVID-19 Genesig Real-Time PCR assay |
| NY State Department of Health | New York SARS-CoV-2 Real-time Reverse |
| (performed at Wadsworth Center | Transcriptase (RT)-PCR Diagnostic Panel |
| and New York City Department | |
| of Health and Mental Hygiene, | |
| Public Health Laboratories) | |
| Orig3n | Orig3n 2019 Novel Coronavirus (COVID-19) Test |
| Ortho Clinical Diagnostics | Vitros Immunodiagnostic Products Anti-SARS-CoV-2 |
| | Total Reagent Pack and Calibrators |
| Osang Healthcare | GeneFinder COVID-19 Plus RealAmp Kit |
| PathoFinder | RealAccurate Quadruplex Corona-plus PCR Kit |
| PCL | COVID19 IgG/IgM Rapid Gold |
| PerkinElmer | PerkinElmer New Coronavirus Nucleic Acid |
| | Detection Kit |
| Phamatech | COVID19 IgG/IgM Rapid Test |
| Promedical | COVID-19 Rapid Test, Wondfo SARS-CoV-2 |
| | Antibody Test (Lateral Flow Method) |
| Qiagen | QiaStat-Dx Respiratory SARS-CoV-2 Panel |
| Quest Diagnostics | Coronavirus Disease 2019 (COVID-19) Test |
| Quidel | Lyra SARS-CoV-2 Assay |
| Rendu Biotechnology | 2019-nCoV detection kit |
| Roche | Cobas SARS-CoV-2 Test |
| Rutgers University Clinical | ThermoFisher - Applied Biosystems TaqPath COVID- |
| Genomics Laboratory | 19 Combo Kit |
| ScienCell Research | ScienCell SARS-CoV-2 Coronavirus Real-time RT- |
| Laboratories | PCR (RT-qPCR) Detection Kit |
| SD Biosensor | Standard Q COVID-19 IgM/IgG Duo |
| Seegene | Allplex 2019-nCoV Assay |
| Sentinel Diagnostics | STAT-NAT COVID-19 HK kit, B kit |
| Shanghai Fosun Long March | novel coronavirus nucleic acid detection kit |
| Medical Science/Shanghai Fosun | |
| Pharmaceutical | |
| Shenzhen Landwind Medical | COVID-19 IgG/IgM Rapid Test Device |
| Snibe Diagnostics | Maglumi 2019-nCoV (SARS-CoV-2) IgM/IgG kits |
| SolGent | DiaPlexQ Novel Coronavirus (2019-nCoV) Detection |
| | kit |
| Specialty Diagnostic (SDI) | SDI SARS-CoV-2 Assay |
| Laboratories | |
| Stanford Health Care Clinical | SARS-CoV-2 PCR Assay |
| Virology Laboratory | |
| SureScreen Diagnostics | SureScreen COVID19 IgM/IgG Rapid Test Cassette |
| Suzhou Kangheshun Medical | SARS-CoV-2 IgG/IgM Rapid Test Cassette |
| Technology | |
| Systaaq Diagnostic Products | 2019-Novel Coronavirus (COVID-19) Real Time PCR |
| | Kit |
| Telepoint Medical Services | SARS-CoV-2 IgG/IgM Rapid Qualitative Test |
| Thermo Fisher Scientific | TaqPath COVID-19 Combo Kit, RT-PCR CE-IVD Kit |
| Tianjin Beroni Biotechnology | SARS-CoV-2 IgG/IgM Antibody Detection Kit |
| TIB Molbiol Syntheselabor | Sarbecovirus E-gene |
| Trax Management Services | Phoenix Dx 2019-CoV |
| United Biomedical | UBI SARS-CoV-2 ELISA |
| University of North Carolina | UNC Health SARS-CoV-2 real- |
| Medical Center | time RT-PCR test |
| Vela Diagnostics | ViroKey SARS-CoV-2 RT-PCR Test |
| Viracor Eurofins | Viracor SARS-CoV-2 assay |
| Vision Medicals | SARS-CoV-2 Clinical Sequencing assay |
| VivaChek Biotech (Hangzhou) | VivaDiag COVID-19 IgM/IgG Rapid Test |
| Yale New Haven Hospital | SARS-CoV-2 PCR test |
| Clinical Virology Laboratory | |
| YD Diagnostics | MolecuTech Real-Time COVID-19 |
| Zhejiang Orient Gene Biotech | COVID-19 IgG/IgM Rapid Test Cassette |
| Zhengzhou Fortune Bioscience | IgG/IgM Antibody Rapid Test Kits (Colloidal Gold |
| | Immunochromatography method) |
| Zhongshan Bio-Tech | SARS-CoV-2 IgM/IgG (GICA) |
| Zhuhai Encode Medical | Novel Coronavirus (COVID-19) IgG/IgM Rapid Test |
| Engineering | Device |
| Zhuhai Livzon Diagnostics | Diagnostic Kit for IgM/IgG Antibody to Coronavirus |
| | (SARS-CoV-2) (Colloidal Gold) |

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the terms “spike protein,” “spike glycoprotein” or “S protein” are used interchangeably, referring to a glycoprotein projecting from the lipid bilayer of the surface of an enveloped virus, such as SARS-COV-2. In some embodiments, an S protein refers to an S protein of a SARS-COV-2. In further embodiments, an S protein or an equivalent thereof as used herein also refers to an S protein variant (for example, an S protein of a naturally occurring SARS-COV-2 variant, such as a delta variant), an S protein mutant (for example, a mutated S protein as disclosed herein), an S protein fragment (such as an immunogenic fragment), or any combination thereof (such as, a naturally occurring variant engineered with additional mutation(s), or a fragment thereof).

In some embodiments, an S protein as used herein comprises, or consists essentially of, or yet further consists of an S1 polypeptide or an S2 polypeptide or both. In some embodiments, an S protein as used herein is a precursor protein comprising, or consisting essentially of, or yet further consisting of both S1 and S2. Such precursor can be processed into S1 and S2 by Cathepsin L (CTSL), Transmembrane Serine Protease (TMPRSS2) or furin to yield the mature S1 and S2 protein. In some embodiments, the S protein as used herein refers to a protein complex comprising, or consisting essentially of, or yet further consisting of a mature S1 protein and a mature S2 protein. In other embodiments, the S protein as used herein refers to an S1 protein. In yet other embodiments, the S protein as used herein refers to an S2 protein. In some embodiments, the precursor protein comprises, or consists essentially of, or yet further consists of the polypeptide as set forth in SEQ ID NO: 1 or an equivalent thereof. In some embodiments, the S1 protein comprises, or consists essentially or, or yet further consists of the polypeptide as set forth in amino acid (aa) 13 to aa 685 of SEQ ID NO: 1 or an equivalent thereof. In some embodiments, the S2 protein comprises, or consists essentially or, or yet further consists of the polypeptide as set forth in amino acid (aa) 686 to aa 1273 of SEQ ID NO: 1 or an equivalent thereof. In some embodiments, the S2 protein comprises, or consists essentially or, or yet further consists of the polypeptide as set forth in amino acid (aa) 816 to aa 1273 of SEQ ID NO: 1 or an equivalent thereof. Further non-limiting exemplary sequences of an S protein or the underlying gene may be found under Gene ID: 43740568 (retrieved from www.ncbi.nlm.nih.gov/gene/43740568, last accessed on Aug. 1, 2021), NCBI Reference Sequence: NC_045512.2 (retrieved from www.ncbi.nlm.nih.gov/nuccore/NC_045512.2/, last accessed on Aug. 1, 2021) or UniProtKB/Swiss-Prot: PODTC2 (retrieved from www.uniprot.org/uniprot/PODTC2, last accessed on Aug. 1, 2021), which are incorporated by reference herein.

In some embodiments, a fragment (such as an immunogenic fragment) of an S protein comprises, or consists essentially of, or yet further consists of a receptor binding domain (RBD) of the S protein. In some embodiments, a receptor-binding domain (RBD) refers to a short immunogenic fragment from a virus that binds to a specific endogenous receptor sequence to gain entry into target cells. In some embodiments, RBD refer to a part of the ‘spike’ glycoprotein (S-domain) which is needed to interact with endogenous receptors to facilitate membrane fusion and delivery to the cytoplasm. In some embodiments, the RBD as used herein comprises, or consists essentially of, or yet further consists of the polypeptide as set forth in aa 319 to aa 541 of SEQ ID NO: 1 or an equivalent thereof.

As used herein the term “angiotensin converting enzyme 2” or “ACE2” refers to an enzyme attached to the membrane of cells optionally located in the intestines, kidney, testis, gallbladder, and heart. ACE2 serves as the entry point into cells for some coronaviruses, including HCoV-NL63, SARS-COV, and SARS-COV-2. The SARS-COV-2 spike protein itself is known to damage the epithelium via downregulation of ACE2. In some embodiments, the term “ACE2” refers to a human ACE2. Non-limiting exemplary sequences of this protein or the underlying gene may be found under Gene Cards ID: GC0XM015494 (retrieved from www.genecards.org/cgi-bin/carddisp.pl?gene=ACE2, last accessed on Aug. 1, 2021), HGNC: 13557 (retrieved from www.genenames.org/data/gene-symbol-report/#!/hgnc_id/13557, last accessed on Aug. 1, 2021), NCBI Entrez Gene: 59272 (retrieved from www.ncbi.nlm.nih.gov/gene/59272, last accessed on Aug. 1, 2021), Ensembl: ENSG00000130234 (retrieved from useast.ensembl.org/Homo_sapiens/Gene/Summary?g=ENSG00000130234; r=X:15494566-15607236, last accessed on Aug. 1, 2021), OMIM®: 300335 (retrieved from omim.org/entry/300335, last accessed on Aug. 1, 2021), or UniProtKB/Swiss-Prot: Q9BYF1 (retrieved from www.uniprot.org/uniprot/Q9BYF1, last accessed on Aug. 1, 2021), which are incorporated by reference herein.

As used herein the term “Transmembrane Serine Protease 2” or “TMPRSS2” refers to protein comprising a type II transmembrane domain, a receptor class A domain, a scavenger receptor cysteine-rich domain and a protease domain. This protein facilitates entry of viruses into host cells by proteolytically cleaving and activating viral envelope glycoproteins. Viruses found to use this protein for cell entry include Influenza virus and the human coronaviruses HCoV-229E, MERS-COV, SARS-COV and SARS-COV-2. In some embodiments, the term “TMPRSS2” refers to a human TMPRSS2. Non-limiting exemplary sequences of this protein or the underlying gene may be found under Gene Cards ID GC21M041464 (retrieved from www.genecards.org/cgi-bin/carddisp.pl?gene=TMPRSS2, last accessed on Aug. 1, 2021), HGNC: 11876 (retrieved from www.genenames.org/data/gene-symbol-report/#!/hgnc_id/11876, last accessed on Aug. 1, 2021), NCBI Entrez Gene: 7113 (retrieved from www.ncbi.nlm.nih.gov/gene/7113, last accessed on Aug. 1, 2021), Ensembl: ENSG00000184012 (retrieved from uscast.ensembl.org/Homo_sapiens/Gene/Summary?g=ENSG00000184012; r=21:41464300-41531116, last accessed on Aug. 1, 2021), OMIM®: 602060 (retrieved from omim.org/entry/602060, last accessed on Aug. 1, 2021), or UniProtKB/Swiss-Prot: 015393 (retrieved from www.uniprot.org/uniprot/015393, last accessed on Aug. 1, 2021), which are incorporated by reference herein.

As used herein the term “Cathepsin L” or “CTSL” refers to a protein, which is a member of the peptidase Cl family, and a dimer composed of disulfide-linked heavy and light chains, both produced from a single protein precursor. Additionally, this protein cleaves the S1 subunit of the SARS-COV-2 spike protein, which is necessary for entry of the virus into the cell. In some embodiments, the term “CTSL” refers to a human CTSL. Non-limiting exemplary sequences of this protein or the underlying gene may be found under Gene Cards ID GC09P087725 (retrieved from www.genecards.org/cgi-bin/carddisp.pl?gene=CTSL, last accessed on Aug. 1, 2021), HGNC: 2537 (retrieved from www.genenames.org/data/gene-symbol-report/#!/hgnc_id/2537, last accessed on Aug. 1, 2021), NCBI Entrez Gene: 1514 (retrieved from www.ncbi.nlm.nih.gov/gene/1514, last accessed on Aug. 1, 2021), Ensembl: ENSG00000135047 (retrieved from useast.ensembl.org/Homo_sapiens/Gene/Summary?g=ENSG00000135047; r=9:87724051-87731469, last accessed on Aug. 1, 2021), OMIM®: 116880 (retrieved from omim.org/entry/116880, last accessed on Aug. 1, 2021), or UniProtKB/Swiss-Prot: P07711 (retrieved from www.uniprot.org/uniprot/P07711, last accessed on Aug. 1, 2021), which are incorporated by reference herein.

As used herein the term “furin” refers to is a protease enzyme capable of cleavage at the RX(K/R)R (RKR or RRR)) consensus motif. It also facilitates SARS-COV-2 infections by proteolytically cleaving the spike protein at the monobasic S1/S2 cleavage site, RRAR (SEQ ID NO: 48). This cleavage is essential for spike protein-mediated cell-cell fusion and entry into human lung cells. In some embodiments, the term furin refers to a mammal furin, such as a bovine furin, see for example UniProtKB Q28193 retrieved from www.uniprot.org/uniprot/Q28193 last accessed on Aug. 10, 2021. In some embodiments, the term “furin” refers to a human furin. Non-limiting exemplary sequences of this protein or the underlying gene may be found under Gene Cards ID: GC15P090868 (retrieved from www.genecards.org/cgi-bin/carddisp.pl?gene=FURIN, last accessed on Aug. 1, 2021), HGNC: 8568 (retrieved from www.genenames.org/data/gene-symbol-report/#!/hgnc_id/8568, last accessed on Aug. 1, 2021), NCBI Entrez Gene: 5045 (retrieved from www.ncbi.nlm.nih.gov/gene/5045, last accessed on Aug. 1, 2021), Ensembl: ENSG00000140564 (retrieved from useast.ensembl.org/Homo_sapiens/Gene/Summary?g=ENSG00000140564; r=15:90868588-90883564, last accessed on Aug. 1, 2021), OMIM®: 136950 (retrieved from omim.org/entry/136950, last accessed on Aug. 1, 2021), or UniProtKB/Swiss-Prot: P09958 (retrieved from www.uniprot.org/uniprot/P09958, last accessed on Aug. 1, 2021), which are incorporated by reference herein.

In some embodiments, a fragment of a protein can be an immunogenic fragment. As used herein, the term “immunogenic fragment” refers to such a polypeptide fragment, which at least partially retains the immunogenicity of the protein from which it is derived. In some embodiments, the immunogenic fragment is at least about 3 amino acid (aa) long, or at least about 4 aa long, or at least about 5 aa long, or at least about 6 aa long, or at least about 7 aa long, or at least about 8 aa long, or at least about 9 aa long, or at least about 10, aa long, or at least about 15, aa long, or at least about 20 aa long, or at least about 25 aa long, or at least about 30 aa long, or at least about 35 aa long, or at least about 40 aa long, or at least about 50 aa long, or at least about 60 aa long, or at least about 70 aa long, or at least about 80 aa long, or at least about 90 aa long, or at least about 100 aa long, or at least about 120 aa long, or at least about 150 aa long, or at least about 200, or longer. In some embodiments, an immunogenic fragment of an S protein comprises, or alternatively consists essentially of, or yet further consists of an RBD of the S protein.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST. In one aspect, equivalent polynucleotides, proteins and corresponding sequences can be determined using BLAST (accessible at blast.ncbi.nlm.nih.gov/Blast.cgi, last accessed on Aug. 1, 2021).

As used herein, an amino acid mutation is referred to herein as two letters separated by an integer, such as T19R. The first letter provides the one letter code of the original amino acid residue to be mutated; while the last letter provides the mutation, such as A indicating a deletion, or one letter code of the mutated amino acid residue. In some embodiments, the integer is the numbering of the to-be-mutated amino acid residue in the amino acid sequence free of the mutation, optionally counting from the N terminus to the C terminus. In some embodiments, the integer is the numbering of the mutated amino acid residue in the mutated amino acid sequence, optionally counting from the N terminus to the C terminus. In some embodiments, the integer is the numbering of the amino acid residue in SEQ ID NO: 1 that corresponds to (such as aligned to) the to-be-mutated residue or the mutated residue or both.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

An equivalent of a reference polypeptide comprises, consists essentially of, or alternatively consists of an polypeptide having at least 80%, or at least 85%, or at least 90%, or at least 95%, or at least about 96%, or at least 97%, or at least 98%, or at least 99% amino acid identity to the reference polypeptide (as determined, in one aspect using the Clustal Omega alignment program), or a polypeptide that is encoded by a polynucleotide that hybridizes under conditions of high stringency to the complementary sequence of a polynucleotide encoding the reference polypeptide, optionally wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “RNA” as used herein refers to its generally accepted meaning in the art. Generally, the term RNA refers to a polynucleotide comprising at least one ribofuranoside moiety. The term can include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, for example at one or more nucleotides of the RNA. Nucleotides in the nucleic acid molecules can also comprise non-standard nucleotides, such as non-naturally occurring nucleotides or chemically synthesized nucleotides or deoxynucleotides. These altered RNAs can be referred to as analogs or analogs of naturally-occurring RNA. In some embodiments, the RNA is a messenger RNA (mRNA).

“Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some embodiments, an mRNA as disclosed herein comprises, or consists essentially of, or yet further consists of at least one coding region, a 5′ untranslated region (UTR), a 3′ UTR, a 5′ cap and a poly-A tail.

Vaccination is the most successful medical approach to disease prevention and control. The successful development and use of vaccines has saved thousands of lives and large amounts of money. A key advantage of RNA vaccines is that RNA can be produced in the laboratory from a DNA template using readily available materials, less expensively and faster than conventional vaccine production, which can require the use of chicken eggs or other mammalian cells. In addition, mRNA vaccines have the potential to streamline vaccine discovery and development, and facilitate a rapid response to emerging infectious diseases, see, for example, Maruggi et al., Mol Ther. 2019; 27(4): 757-772.

Preclinical and clinical trials have shown that mRNA vaccines provide a safe and long-lasting immune response in animal models and humans. mRNA vaccines against infectious diseases may be developed as prophylactic or therapeutic treatments. mRNA vaccines expressing antigens of infectious pathogens have been shown to induce potent T cell and humoral immune responses. See, for example, Pardi et al., Nat Rev Drug Discov. 2018; 17:261-279. The production procedure to generate mRNA vaccines is cell-free, simple, and rapid, compared to production of whole microbe, live attenuated, and subunit vaccines. This fast and simple manufacturing process makes mRNA a promising bio-product that can potentially fill the gap between emerging infectious disease and the desperate need for effective vaccines.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, or protein, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, or protein, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “gene” refers to a polynucleotide containing at least one open reading frame (ORF) that is capable of encoding a particular polypeptide or protein after being transcribed and translated.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. In some embodiments, the term refers to the ribonucleotide modifications in naturally occurring 5′-terminal mRNA cap moieties. In further embodiments, the chemical modification is selected from pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, or 2′-O-methyl uridine. In some embodiments the extent of incorporation of chemically modified nucleotides has been optimized for improved immune responses to the vaccine formulation. In other embodiments, the term excludes the ribonucleotide modifications in naturally occurring 5′-terminal mRNA cap moieties.

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of residues of the RNA is chemically modified by one or more of modifications as disclosed herein. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of uridine residues of the RNA is chemically modified by one or more of modifications as disclosed herein.

In some embodiments, an RNA as disclosed herein is optimized. Optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide.

A “3′ untranslated region” (3′ UTR) refers to a region of an mRNA that is directly downstream (i.e., 3′) from the stop codon (i.e., the codon of an mRNA transcript that signals a termination of translation) that does not encode a polypeptide. In some embodiments, a 3′ UTR as used herein comprises, or consists essentially of, or yet further consists of one or more of SEQ ID NOs: 18, 22, or 24.

A “5′ untranslated region” (5′ UTR) refers to a region of an RNA that is directly upstream (i.e., 5′) from the start codon (i.e., the first codon of an mRNA transcript translated by a ribosome) that does not encode a polypeptide. In some embodiments, a 5′ UTR as used herein comprises, or consists essentially of, or yet further consists of one or both of SEQ ID NO: 20 or 26.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. Additionally or alternatively, in a relevant biological setting (e.g., in cells, in vivo) the polyA tail functions to protect mRNA from enzymatic degradation, e.g., in the cytoplasm, and aids in transcription termination, export of the mRNA from the nucleus and translation. In some embodiments, a polyA tail as used herein comprises, or consists essentially of, or yet further consists of one or more of: SEQ ID NOs: 27, 28, 30, or 56-57.

In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et al., Methods Mol Biol. 703:29-41(2011); Rio et al. RNA: A Laboratory Manual. Cold Spring Harbor: Cold Spring Harbor Laboratory Press, 2011, 205-220; and Cooper, Geoffery M. The Cell: A Molecular Approach. 4th ed. Washington D.C.: ASM Press, 2007, 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that an RNA as disclosed herein can be made using any appropriate synthesis method. For example, in some embodiments, an RNA is made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., a T7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

“Under transcriptional control”, which is also used herein as “directing expression of” or any grammatical variation thereof, is a term well understood in the art and indicates that transcription and optionally translation of a polynucleotide sequence, usually a DNA sequence, depends on its being operatively linked to an element which contributes to the initiation of, or promotes, transcription.

“Operatively linked” intends the polynucleotides are arranged in a manner that allows them to function in a cell.

The term “a regulatory sequence”, “an expression control element” or “promoter” as used herein, intends a polynucleotide that is operatively linked to a target polynucleotide to be transcribed or replicated, and facilitates the expression or replication of the target polynucleotide.

A promoter is an example of an expression control element or a regulatory sequence. Promoters can be located 5′ or upstream of a gene or other polynucleotide, that provides a control point for regulated gene transcription. In some embodiments, a promoter as used herein is corresponding to the RNA polymerase. In further embodiments, a promoter as sued herein comprises, or consists essentially of, or yet further consists of a T7 promoter, or a SP6 promoter, or a T3 promoter. Non-limiting examples of suitable promoters are provided in WO2001009377A1.

An “RNA polymerase” refers to an enzyme that produces a polyribonucleotide sequence, complementary to a pre-existing template polynucleotide (DNA or RNA). In some embodiments, the RNA polymerase is a bacteriophage RNA polymerase, optionally a T7 RNA polymerase, or a SP6 RNA polymerase, or a T3 RNA polymerase. Non-limiting examples of suitable polymerase are further detailed in U.S. Ser. No. 10/526,629B2.

In some embodiments, the term “vector” intends a recombinant vector that retains the ability to infect and transduce non-dividing and/or slowly-dividing cells and optionally integrate into the target cell's genome. Non-limiting examples of vectors include a plasmid, a nanoparticle, a liposome, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. In one embodiment, the viral vector is a retroviral vector. In one embodiment, the vector is a plasmid. In one embodiment, the vector is a nanoparticle, optionally a polymeric nanoparticle or a lipid nanoparticle.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide to be delivered into a host cell, either in vivo, ex vivo or in vitro. As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. Sec, Schlesinger and Dubensky (1999) Curr. Opin. Biotechnol. 5:434-439 and Ying, et al. (1999) Nat. Med. 5(7):823-827. As used herein, Multiplicity of infection (MOI) refers to the number of viral particles that are added per cell during infection.

A retrovirus such as a gammaretrovirus and/or a lentivirus comprises (a) envelope comprising lipids and glycoprotein, (b) a vector genome, which is a RNA (usually a dimer RNA comprising a cap at the 5′ end and a polyA tail at the 3′ end flanked by LTRs) derived to the target cell, (c) a capsid, and (d) proteins, such as a protease. U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters.

A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, the identity is calculated between two peptides or polynucleotides over their full-length, or over the shorter sequence of the two, or over the longer sequence of the two.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: blast.ncbi.nlm.nih.gov/Blast.cgi, last accessed on Aug. 1, 2021.

In some embodiments, the polynucleotide as disclosed herein is a RNA or an analog thereof. In some embodiments, the polynucleotide as disclosed herein is a DNA or an analog thereof. In some embodiments, the polynucleotide as disclosed herein is a hybrid of DNA and RNA or an analog thereof.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complementary. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” refers to the process whereby a foreign nucleotide sequence is introduced into a cell. In some embodiments, this transduction is done via a vector, viral or non-viral.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, protein or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, cosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

A “selection marker” refers to a protein or a gene encoding the protein necessary for survival or growth of a cell grown in a selective culture regimen. Typical selection markers include sequences that encode proteins, which confer resistance to selective agents, such as antibiotics, herbicides, or other toxins. Examples of selection markers include genes for conferring resistance to antibiotics, such as spectinomycin, streptomycin, tetracycline, ampicillin, kanamycin, G 418, neomycin, bleomycin, hygromycin, methotrexate, dicamba, glufosinate, or glyphosate.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

In some embodiments, the cell as disclosed herein is a eukaryotic cell or a prokaryotic cell. In some embodiments, the cell is a human cell. In some embodiments, the cell is a cell line, such as a human embryonic kidney 293 cell (HEK 293 cell or 293 cell), a 293T cell, or an a549 cell.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The host cell can be a prokaryotic or a eukaryotic cell. In some embodiments, the host cell is a cell line, such as a human embryonic kidney 293 cell (HEK 293 cell or 293 cell), a 293T cell, or an a549 cell.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, canine, bovine, porcine, murine, rat, avian, reptilian and human.

“Prokaryotic cells” that usually lack a nucleus or any other membrane-bound organelles and are divided into two domains, bacteria and archaca. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for case of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A unit dose, dosage, or regimen can be determined from the IC50 of a given polynucleotide, vector, cell, composition, or kit, for neutralizing activity against a SARS-CoV-2 polypeptide or polynucleotide, preferably a SARS-COV-2 spike protein. “IC50” means the concentration of polynucleotide, vector, cell, composition, or kit required for 50% inhibition. Alternatively, the effective amount can be determined from the EC50 of a given polynucleotide, vector, cell, composition, or kit. “EC50” means the plasma concentration required to obtain 50% of the maximum neutralizing effect in vivo.

A combination as used herein intends that the individual active ingredients of the compositions are separately formulated for use in combination, and can be separately packaged with or without specific dosages. The active ingredients of the combination can be administered concurrently or sequentially.

The four-branched histidine-lysine (HK) peptide polymer H2K4b has been shown to be a good carrier of large molecular weight DNA plasmids (Leng et al. Nucleic Acids Res 2005; 33:e40.), but a poor carrier of relatively low molecular weight siRNA (Leng et al. J Gene Med 2005; 7:977-986). Two histidine-rich peptides analogs of H2K4b, namely H3K4b and H3K(+H)4b, were shown to be effective carriers of siRNA (Leng et al. J Gene Med 2005; 7: 977-986. Chou et al. Biomaterials 2014; 35:846-855), although H3K(+H)4b appeared to be modestly more effective (Leng et al. Mol Ther 2012; 20:2282-2290). Moreover, the H3K4b carrier of siRNA induced cytokines to a significantly greater degree in vitro and in vivo than H3K(+H)4b siRNA polyplexes (Leng et al. Mol Ther 2012; 20:2282-2290). Suitable HK polypeptides are described in WO/2001/047496, WO/2003/090719, and WO/2006/060182, the contents of each of which are incorporated herein in their entireties. These polypeptides have a lysine backbone (three lysine residues) where the lysine side chain ε-amino groups and the N-terminus are coupled to various HK sequences. HK polypeptide carriers can be synthesized by methods that are well-known in the art including, for example, solid-phase synthesis.

It was found that such histidine-lysine peptide polymers (“HK polymers” or “HKP”) were surprisingly effective as mRNA carriers, and that they can be used, alone or in combination with liposomes, to provide effective delivery of mRNA into target cells. Similar to PEI and other carriers, initial results suggested HK polymers differ in their ability to carry and release nucleic acids. However, because HK polymers can be reproducibly made on a peptide synthesizer, their amino acid sequence can be easily varied, thereby allowing fine control of the binding and release of RNAs, as well as the stability of polyplexes containing the HK polymers and RNA (Chou et al. Biomaterials 2014; 35:846-855. Midoux et al. Bioconjug Chem 1999; 10:406-411. Henig et al. Journal of American Chemical Society 1999; 121:5123-5126). When mRNA molecules are admixed with one or more HKP carriers the components self-assemble into nanoparticles.

As described herein, advantageously the HK polymer comprises four short peptide branches linked to a three-lysine amino acid core. The peptide branches consist of histidine and lysine amino acids, in different configurations. The general structure of these histidine-lysine peptide polymers (HK polymers) is shown in Formula I, where R represents the peptide branches and K is the amino acid L-lysine.

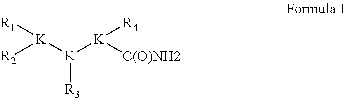

In Formula I where K is L-lysine and each of R₁, R₂, R₃ and R₄ is independently a histidine-lysine peptide. The R₁-4 branches may be the same or different in the HK polymers of the invention. When a R branch is “different”, the amino acid sequence of that branch differs from each of the other R branches in the polymer. Suitable R branches used in the HK polymers of the invention shown in Formula I include, but are not limited to, the following R branches R_{A}-R_{-J}:

(SEQ ID NO: 38)
R_{A} = KHKHHKHHKHHKHHKHHKHK-
(SEQ ID NO: 39)
R_{B} = KHHHKHHHKHHHKHHHK-
(SEQ ID NO: 40)
R_{C} = KHHHKHHHKHHHHKHHHK-
(SEQ ID NO: 41)
R_{D} = kHHHkHHHkHHHHkHHHk-
(SEQ ID NO: 42)
R_{E} = HKHHHKHHHKHHHHKHHHK-
(SEQ ID NO: 43)
R_{F} = HHKHHHKHHHKHHHHKHHHK-
(SEQ ID NO: 44)
R_{G} = KHHHHKHHHHKHHHHKHHHHK-
(SEQ ID NO: 45)
R_{H} = KHHHKHHHKHHHKHHHHK-
(SEQ ID NO: 46)
R_{I} = KHHHKHHHHKHHHKHHHK-
(SEQ ID NO: 47)
R_{J} = KHHHKHHHHKHHHKHHHHK-

Specific HK polymers that may be used in the mRNA compositions include, but are not limited to, HK polymers where each of R₁, R₂, R₃ and R₄ is the same and selected from R_{A}-R_{J} (Table 1). These HK polymers are termed H2K4b, H3K4b, H3K(+H)4b, H3k(+H)4b, H-H3K(+H)4b, HH-H3K(+H)4b, H4K4b, H3K(1+H)4b, H3K(3+H)4b and H3K(1,3+H)4b, respectively. In each of these 10 examples, upper case “K” represents a L-lysine, and lower case “k” represents D-lysine. Extra histidine residues, in comparison to H3K4b, are underlined within the branch sequences. Nomenclature of the HK polymers is as follows:
1) for H3K4b, the dominant repeating sequence in the branches is -HHHK- (SEQ ID NO: 49), thus “H3K” is part of the name; the “4b” refers to the number of branches;
2) there are four -HHHK- (SEQ ID NO: 49) motifs in each branch of H3K4b and analogues; the first -HHHK- motif (SEQ ID NO: 49) (“1”) is closest to the lysine core;
3) H3K(+H)4b is an analogue of H3K4b in which one extra histidine is inserted in the second -HHHK- motif (SEQ ID NO: 49) (motif 2) of H3K4b;
4) for H3K(1+H)4b and H3K(3+H)4b peptides, there is an extra histidine in the first (motif 1) and third (motif 3) motifs, respectively;
5) for H3K(1,3+H)4b, there are two extra histidines in both the first and the third motifs of the branches.

TABLE 1

| | | Sequence |
|---|---|---|
| Polymer | Branch Sequence | Identifier |
| H2K4b | R_{A} = KHKHHKHHKHHKHHKHHKHK- | (SEQ ID |
| | | NO: 38) |
| | 4 3 2 1 | |
| H3K4b | R_{B} = KHHHKHHHKHHHKHHHK - | (SEQ ID |
| | | NO: 39) |
| H3K(+H)4b | R_{C} = KHHHKHHHKHHHHKHHHK- | (SEQ ID |
| | | NO: 40) |
| H3k(+H)4b | R_{D} = kHHHkHHHkHHHHkHHHk- | (SEQ ID |
| | | NO: 41) |
| H- | R_{E} = HKHHHKHHHKHHHHKHHHK- | (SEQ ID |
| H3K(+H)4b | | NO: 42) |
| HH- | R_{F} = HHKHHHKHHHKHHHHKHHHK- | (SEQ ID |
| H3K(+H)4b | | NO: 43) |
| H4K4b | R_{G} = KHHHHKHHHHKHHHHKHHHHK- | (SEQ ID |
| | | NO: 44) |
| H3K | R_{H} = KHHHKHHHKHHHKHHHHK- | (SEQ ID |
| (1+H)4b | | NO: 45) |
| H3K | R_{I} = KHHHKHHHHKHHHKHHHK- | (SEQ ID |
| (3+H)4b | | NO: 46) |
| H3K | R_{J} = KHHHKHHHHKHHHKHHHHK- | (SEQ ID |
| (1,3+H)4b | | NO: 47) |

Methods well known in the art, including gel retardation assays, heparin displacement assays and flow cytometry can be performed to assess performance of different formulations containing HK polymer plus liposome in successfully delivering mRNA. Suitable methods are described in, for example, Gujrati et al, Mol. Pharmaceutics 11:2734-2744 (2014), and Pärnaste et al., Mol Ther Nucleic Acids. 7: 1-10 (2017).

Detection of mRNA uptake into cells can also be achieved using SMARTFLARE® technology (Millipore Sigma). These smart flares are beads that have a sequence attached that, when recognizing the RNA sequence in the cell, produce an increase in fluorescence that can be analyzed with a fluorescent microscope.

Other methods include measuring protein expressions from an mRNA, for example, an mRNA encoding luciferase can be used to measure the efficiency of transfection. See, for example, He et al (J Gene Med. 2021 February; 23(2):e3295) demonstrating the efficacy of delivering mRNA using a luciferase model HKP and liposome formulation.

In the luciferase model as described above, the combination of H3K(+H)4b and DOTAP (a cationic lipid) surprisingly was synergistic in its ability to carry mRNA into MDA-MB-231 cells (H3K(+H)4b/liposomes vs liposomes, P<0.0001). The combination was about 3-fold and 8-fold more effective as carriers of mRNA than the polymer alone and the cationic lipid carrier, respectively. Not all HK peptides demonstrated the synergistic activity with DOTAP lipid. For example, the combination of H3K4b and DOTAP was less effective than the DOTAP liposomes as carriers of luciferase mRNA. Besides DOTAP, other cationic lipids that may be used with HK peptides include Lipofectin (ThermoFisher), Lipofectamine (ThermoFisher), and DOSPER.

In the luciferase model as described above, the D-isomer of H3k (+H)4b, in which the L-lysines in the branches are replaced with D-lysines, was the most effective polymeric carrier (H3k(+H)4b vs. H3K(+H)4b, P<0.05). The D-isomer/liposome carrier of mRNA was nearly 4-fold and 10-fold more effective than the H3k(+H)4b alone and liposome carrier, respectively. Although the D-H3k(+H)4b/lipid combination was modestly more effective than the L-H3K(+H)4b/lipidmbination, this comparison was not statistically different.

Both H3K4b and H3K(+H)4b can be used as carriers of nucleic acids in vitro See, for example, Leng et al. J Gene Med 2005; 7: 977-986; and Chou et al., Cancer Gene Ther 2011; 18: 707-716. Despite these previous findings, H3K(+H)4b was markedly better as a carrier of mRNA compared to other similar analogues in the luciferase model (Table 2).

TABLE 2

| Polymer | Ratio(wt:wt; mRNA:Polymer) | RLU/ug-Protein |
|---|---|---|
| H3K(+H)4b | 1:4 | 1532.9 ± 122.9 |
| | 1:8 | 1656.3 ± 202.5 |
| | 1:12 | 1033.4 ± 197 |
| H3k(+H)4b | 1:4 | 1851.6 ± 138.3 |
| | 1:8 | 1787.2 ± 195.2 |
| | 1:12 | 1982.3 ± 210.7 |
| H3K4b | 1:4 | 156.8 ± 41.8 |
| | 1:8 | 62.1 ± 13.2 |
| | 1:12 | 18.1 ± 4.0 |
| H3K(3 + H)4b | 1:4 | 61.7 ± 5.7 |
| | 1:8 | 68.7 ± 3.1 |
| | 1:12 | 59.0 ± 7.5 |
| H3K(1 + H)4b | 1:4 | 24.3 ± 4.5 |
| | 1:8 | 15.0 ± 3.6 |
| | 1:12 | 7.3 ± 2.5 |
| H-H3K(+H)4b | 1:4 | 1107.5 ± 140.4 |
| | 1:8 | 874.6 ± 65.2 |
| | 1:12 | 676.4 ± 25.7 |
| HH-H3K(+H)4b | 1:4 | 1101.9 ± 106.6 |
| | 1:8 | 832.2 ± 75.3 |
| | 1:12 | 739.8 ± 105.4 |
| H4K4b | 1:4 | 896.4 ± 112.6 |
| | 1:8 | 821.8 ± 115.6 |
| | 1:12 | 522.4 ± 69.2 |
| H3K(1,3 + H)4b | 1:4 | 518.3 ± 134.7 |
| | 1:8 | 427.7 ± 18.1 |
| | 1:12 | 378 ± 5.2 |
| H2K4b | 1:4 | 546.7 ± 70.1 |
| | 1:8 | 132.3 ± 58.5 |
| | 1:12 | 194.7 ± 18.4 |

Especially, it has higher mRNA transfection efficiency than H3K4b in various weight:weight (HK:mRNA) ratios. At a 4:1 ratio, luciferase expression was 10-fold higher with H3K(+H)4b than H3K4b in MDA-MB-231 cells without significant cytotoxicity. Moreover, the buffering capacity does not seem to be an essential factor in their transfection differences since the percent of histidines (by weight) in H3K4b and H3K(+H)4b is 68.9 and 70.6%, respectively.

Gel retardation assays show that the electrophoretic mobility of mRNA was delayed by the HK polymers. The retardation effect increased with higher peptide to mRNA weight ratios. However, mRNA was completely retarded in 2:1 ratio of H3K(+H)4b, whereas it was not completely retarded by H3K4b. This suggested that H3K(+H)4b could form a more stable polyplex, which was advantageous for its ability to be a suitable carrier for mRNA delivery.

Further confirmation that the H3K(+H)4b peptide binds more tightly to mRNA was demonstrated with a heparin-displacement assay. Various concentrations of heparin was added into the polyplexes formed with mRNA and HK and it was observed that, particularly at the lower concentrations of heparin, mRNA was released by the H3K4b polymer more readily than the H3K(+H)4b polymer. These data suggest H3K(+H)4b could bind to mRNA and form a more stable polyplex than H3K4b.

With the luciferase mRNA labeled with cyanine-5, the uptake of H3K4b and H3K(+H)4b polyplexes into MDA-MB-231 cells was compared using flow cytometry. At different time points (1, 2, and 4 h), the H3K(+H)4b polyplexes were imported into the cells more efficiently than H3K4b polyplexes. Similar to these results, fluorescent microscopy indicated that H3K(+H)4b polyplexes localized within the acidic endosomal vesicles significantly more than H3K4b polyplexes (H3K4b vs. H3K(+H)4b, P<0.001). Interestingly, irregularly-shaped H3K4b polyplexes, which did not overlap endocytic vesicles, were likely extracellular and were not observed with H3K(+H)4b polyplexes.

It is known both that HK polymers and cationic lipids (i.e., DOTAP) significantly and independently increase transfection with plasmids. See, for example, Chen et al. Gene Ther 2000; 7: 1698-1705. Consequently, whether these lipids together with HK polymers enhanced mRNA transfection was investigated. Notably, the H3K(+H)4b and H3k(+H)4b carriers were significantly better carriers of mRNA than the DOTAP liposomes. The combination of H3K(+H)4b and DOTAP lipid was synergistic in the ability to carry mRNA into MDA-MB-231 cells. The combination was about 3-fold and 8-fold more effective as carriers of mRNA than the polymer alone and the liposome carrier, respectively (H3K(+H)4b/lipid vs. liposomes or H3K(+H)4b). Notably, not all HK peptides demonstrated improved activity with DOTAP lipid. The combination of H3K4b and DOTAP carriers was less effective than the DOTAP liposomes as carriers of luciferase mRNA. The combination of DOTAP and H3K(+H)4b carriers were found to be synergistic in their ability to carry mRNA into cells. Sec, for example, He et al. J Gene Med. 2020 Nov. 10:03295.

In some embodiments, the carrier, such as the NKP nanoparticle, further comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA, or MC3), and di((Z)-non-2-en-1-yl) 9-((4-(dimethylamino)butanoyl)oxy)heptadecanedioate (L319).

In some embodiments, the carrier is a lipid nanoparticle (LNP). In some embodiments, the LNP has a mean diameter of about 50 nm to about 200 nm. In some embodiments, Lipid nanoparticle carriers/formulations typically comprise, or alternatively consist essentially of, or yet further consist of a lipid, in particular, an ionizable cationic lipid, for example, SM-102 as disclosed herein, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319). In some embodiments, the LNP carriers/formulations further comprise a neutral lipid, a sterol (such as a cholesterol) and a molecule capable of reducing particle aggregation, for example a PEG or PEG-modified lipid (also referred to herein as PEGylated lipid). Additional exemplary lipid nanoparticle compositions and methods of making same are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21:1570-1578, the contents of each of which are incorporated herein by reference in their entirety.

In one embodiment, the term “disease” or “disorder” as used herein refers to a SARS-COV-2 infection, a status of being diagnosed with a SARS-COV-2 infection, a status of being suspect of having a SARS-COV-2 infection, or a status of at high risk of having a SARS-COV-2 infection. In one embodiment, the term “disease” or “disorder” as used herein refers to a symptomatic SARS-COV-2 infection, a status of being diagnosed with a symptomatic SARS-COV-2 infection, a status of being suspect of having a symptomatic SARS-COV-2 infection, or a status of at high risk of having a symptomatic SARS-COV-2 infection.

In one embodiment, the term “disease” or “disorder” as used herein refers to COVID-19, a status of being diagnosed with COVID-19, a status of being suspect of having COVID-19, or a status of at high risk of having COVID-19. In one embodiment, the term “disease” or “disorder” as used herein refers to a symptomatic COVID-19, a status of being diagnosed with a symptomatic COVID-19, a status of being suspect of having a symptomatic COVID-19, or a status of at high risk of having a symptomatic COVID-19.

As used herein, the term “animal” refers to living multi-cellular vertebrate organisms, a category that includes, for example, mammals and birds. The term “mammal” includes both human and non-human mammals such as non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, bat, rat, rabbit, guinea pig).

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, bat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

In some embodiments, the terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, “viral load”, also known as “viral burden,” “viral titer”, “viral level” or “viral expression” in some embodiments, is a measure of the severity of a viral infection, and can be calculated by estimating the amount of virus in an infected organism, an involved body fluid, or a biological sample.

As used herein, a biological sample, or a sample, is obtained from a subject. Exemplary samples include, but are not limited to, cell sample, tissue sample, biopsy, liquid samples such as blood and other liquid samples of biological origin, including, but not limited to, anterior nasal swab, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, car, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

The term “adjuvant” refers to a substance or mixture that enhances the immune response to an antigen. As non-limiting example, the adjuvant can comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (See e.g., U.S. Pat. No. 8,241,610). In another embodiment, the synthetic nanocarrier may comprise at least one polynucleotide and an adjuvant. As a non-limiting example, the synthetic nanocarrier comprising and adjuvant can be formulated by the methods described in WO2011150240 and US20110293700, each of which is herein incorporated by reference in its entirety.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a polynucleotide, vector, cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include inhalation, intramuscular administration, nasal administration, oral administration, intraperitoneal, infusion, injection, and topical application. In preferred embodiments, the route of administration is inhalation or intramuscular administration. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

In some embodiments, an RNA, polynucleotide, vector, cell or composition as disclosed herein is administered in an effective amount. An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

As used herein, the term “RL-007” and RL007″ refers to an lipid nanoparticle formulation or mix that is prepared by mixing a final concentration of 6.25 mM of SM-102, 1.25 mM of DSPC, 4.815 mM of Cholesterol, and 0.1875 of mM DMG-PEG2000 (i.e., a 50:10:38:1.5 molar ratio). The Terms “RL-007 vaccine”, “RL007 vaccine”, “RL-007 mRNA vaccine”, or “RL007 mRNA vaccine” refer to a vaccine which comprises, or alternatively consists essentially of, or yet further consists of an RL-007 carrier. In some embodiments, the pharmaceutically acceptable carrier comprises, or alternatively consists essentially of, or yet further consists of RL-007.

### Modes for Carrying Out the Disclosure

### RNA

The disclosure herein provides a ribonucleic acid (RNA) or DNA encoding a spike (S) protein or a fragment thereof (such as an immunogenic fragment) of a severe acute respiratory syndrome coronavirus 2 (SARS-COV-2). The S protein or fragment comprises, or consists essentially of, or yet further consists of, at least one non-naturally occurring amino acid mutation.

In some embodiments, the fragment or immunogenic fragment comprises, or alternatively consists essentially of, or yet further consists of a receptor binding domain (RBD) of the S protein or an equivalent thereof, such as a fragment corresponding (such as aligning) to aa 319 to aa 541 of SEQ ID NO: 1. In some embodiments, a receptor-binding domain (RBD) refers to a short immunogenic fragment from a virus that binds to a specific endogenous receptor sequence to gain entry into target cells. In some embodiments, RBD refer to a part of the ‘spike’ glycoprotein which is needed to interact with endogenous receptors to facilitate membrane fusion and delivery to the cytoplasm. In some embodiments, the RBD as used herein comprises, or consists essentially of, or yet further consists of the polypeptide as set forth in aa 319 to aa 541 of SEQ ID NO: 1 or an equivalent thereof. In some embodiments, the fragment or immunogenic fragment is at least about 5 amino acids long, or at least about 8 amino acids long, or at least about 10 amino acids long, or at least about 15 amino acids long, or at least about 20 amino acids long, or at least about 25 amino acids long, or at least about 30 amino acids long, or at least about 40 amino acids long, or at least about 50 amino acids long, or at least about 60 amino acids long, or at least about 70 amino acids long, or at least about 80 amino acids long, or at least about 100 amino acids long, or at least about 125 amino acids long, or at least about 150 amino acids long, or at least about 160 amino acids long, or at least about 170 amino acids long, or at least about 180 amino acids long, or at least about 190 amino acids long, or at least about 200 amino acids long, or at least about 250 amino acids long, or at least about 300, or longer; and optionally comprises, consists essentially of, or yet further consists of a RBD of the S protein or an equivalent thereof. The immunogenic fragment is useful for inducing an immune response to the SARS-COV-2, or reducing or inhibiting the binding of SARS-COV-2 to its receptor, such as ACE2, or both and a fragment that is non-immunogenic is useful as a control in the assays as provided herein.

In some embodiments, the at least one non-naturally occurring amino acid mutation comprises, or alternatively consists essentially of, or yet further consists of a mutation in a furin-like cleavage site. In further embodiments, the furin-like cleavage site comprises, or alternatively consists of, or yet further consists of RRAR (SEQ ID NO: 48). In yet further embodiments, a furin-like cleavage site in an S protein or a fragment thereof as disclosed herein corresponds to (e.g., aligns to) amino acid (aa) 682 to aa 685 of SEQ ID NO: 1. In some embodiments, the at least one non-naturally occurring amino acid mutation comprises, or alternatively consists essentially of, or yet further consists of one or more of: a serine (S) as the amino acid corresponding to R682 of SEQ ID NO: 1 (R682S), an S as the amino acid corresponding to R683 of SEQ ID NO: 1 (R683S), a glycine (G) as the amino acid corresponding to R685 of SEQ ID NO: 1 (R685G), or a G as the amino acid corresponding to R682 of SEQ ID NO: 1 (R682G). In some embodiments, the at least one non-naturally occurring amino acid mutation comprises, or alternatively consists essentially of, or yet further consists R682S and R685G. In some embodiments, a mutated furin-like cleavage site stabilize the S protein or fragment thereof as disclosed herein.

Additionally or alternatively, the at least one non-naturally occurring amino acid mutation comprises, or alternatively consists essentially of, or yet further consists of a proline (P) as the amino acid corresponding to any one or more (such as any two, or any three, or any four, or any five, or all six) of F817 of SEQ ID NO: 1 (F817P), A892 of SEQ ID NO: 1 (A892P), A899 of SEQ ID NO: 1 (A899P), A942 of SEQ ID NO: 1 (A942P), K986 of SEQ ID NO: 1 (K986P), or V987 of SEQ ID NO: 1 (V987P). In some embodiments, the at least one non-naturally occurring amino acid mutation comprises, or alternatively consists essentially of, yet further consists of K986P and V987P. These two mutations are referred to herein as S2P. In some embodiments, the at least one non-naturally occurring amino acid mutation comprises, or alternatively consists essentially of, yet further consists of F817P, A892P, A899P, A942P, K986P, and V987P. These six mutations are referred to herein as “S-HexaPro” or “S6P”. In some embodiments, an S protein or a fragment (such as an immunogenic fragment thereof) comprising the S2P or S6P mutations has one or more of the following properties compared to those without the S2P or S6P mutations: increased expression level in vivo or in vitro or both; higher stability, such as room temperature, under heat, or after freeze-thaws; or maintaining the protein conformation.

In some embodiments, the at least one non-naturally occurring amino acid mutation comprises, or alternatively consists essentially of, or yet further consists of one or more of: a serine (S) as the amino acid corresponding to R682 of SEQ ID NO: 1 (R682S), a glycine (G) as the amino acid corresponding to R685 of SEQ ID NO: 1 (R685G), a proline (P) as the amino acid corresponding to F817 of SEQ ID NO: 1 (F817P), a P as the amino acid corresponding to A892 of SEQ ID NO: 1 (A892P), a P as the amino acid corresponding to A899 of SEQ ID NO: 1 (A899P), a P as the amino acid corresponding to A942 of SEQ ID NO: 1 (A942P), a P as the amino acid corresponding to K986 of SEQ ID NO: 1 (K986P), or a P as the amino acid corresponding to V987 of SEQ ID NO: 1 (V987P).

Without wishing to be bound by the theory, an S protein or a fragment thereof comprises a mutated furin-like cleavage site and S2P or S6P mutations shows an advantage (such as an higher expression level in vivo and in vitro, or a better stability, or both) over those free of these mutations, or comprising a mutated furin-like cleavage site alone, or comprising S2P or S6P alone. In some embodiments, the advantage of comprising both mutation sets is synergistic.

In some embodiments, the S protein or fragment thereof is derived from a naturally occurring SARS-COV-2 variant, such as an alpha variant, a beta variant, a delta variant, or a gamma variant. For example, the at least one non-naturally occurring amino acid mutations as disclosed herein can be engineered to a S protein variant or a fragment thereof, such as an alpha variant, a beta variant, a delta variant, or a gamma variant, thus arriving at a mutated S protein or a fragment thereof.

In further embodiments, the S protein or fragment thereof is derived from a chimeric SARS-COV-2 S protein. The chimeric S protein comprises, or alternatively consists essentially of, or yet further consists of a first naturally occurring S variant having one or more of its amino acids or continuous segments substituted with the corresponding amino acids or continuous segments of a second naturally occurring S variant.

In some embodiments, the S protein or fragment thereof further comprises one or more mutations, which was found in a naturally occurring SARS-COV-2 variant, such as a delta variant. In some embodiments, the one or more mutations comprises, or alternatively consists essentially of, or yet further consists of one or both of: a lysine (K) as the amino acid corresponding to N440 of SEQ ID NO: 1 (N440K), or a K as the amino acid corresponding to E484 of SEQ ID NO: 1 (E484K). Additionally or alternatively, the one or more mutations comprises, or alternatively consists essentially of, or yet further consists of an arginine (R) as the amino acid corresponding to T19 of SEQ ID NO: 1 (T19R), a phenylalanine (F) as the amino acid corresponding to V70 of SEQ ID NO: 1 (V70F), an isoleucine (I) as the amino acid corresponding to T95 of SEQ ID NO: 1 (T95I), an aspartic acid (D) as the amino acid corresponding to G142 of SEQ ID NO: 1 (G142D), a deletion corresponding to E156 of SEQ ID NO: 1 (E156Δ), a deletion corresponding to F157 of SEQ ID NO: 1 (F157Δ), a G as the amino acid corresponding to R158 of SEQ ID NO:1 (R158G), a valine (V) as the amino acid corresponding to A222 of SEQ ID NO: 1 (A222V), a leucine (L) as the amino acid corresponding to W258 of SEQ ID NO: 1 (W258L), an asparagine (N) as the amino acid corresponding to K417 of SEQ ID NO: 1 (K417N), an R as the amino acid corresponding to K417 of SEQ ID NO: 1 (L452R), a K as the amino acid corresponding to T478 of SEQ ID NO: 1 (T478K), a G as the amino acid corresponding to D614 of SEQ ID NO: 1 (D614G), an R as the amino acid corresponding to P681 of SEQ ID NO: 1 (P681R), or an N as the amino acid corresponding to D950 of SEQ ID NO: 1 (D950N). In some embodiments, the one or more mutations comprises, or alternatively consists essentially of, or yet further consists of T19R, T95I, G142D, E156Δ, F157Δ, R158G, L452R, T478K, D614G, P681R and D950N. In some embodiments, the one or more mutations comprises, or alternatively consists essentially of, or yet further consists of T19R, T95I, G142D, E156Δ, F157Δ, R158G, L452R, T478K, D614G and D950N. In some embodiments, the one or more mutations comprises, or alternatively consists essentially of, or yet further consists of T19R, V70F, T95I, G142D, E156Δ, F157Δ, R158G, A222V, W258L, K417N, L452R, T478K, D614G, P681R and D950N. In some embodiments, the one or more mutations comprises, or alternatively consists essentially of, or yet further consists of T19R, V70F, T95I, G142D, E156Δ, F157Δ, R158G, A222V, W258L, K417N, N440K, LA52R, T478K, E484K, D614G, P681R and D950N.

In some embodiments, the S protein comprises, or alternatively consists essentially of, or yet further consists of the polypeptide of SEQ ID NO: 5 or an equivalent thereof. In some embodiments, the equivalent of SEQ ID NO: 5 retains the mutations of SEQ ID NO: 5, i.e., T19R, T95I, G142D, E156Δ, F157Δ, R158G, L452R, T478K, D614G, P681R, R682S, R685G, D950N, K986P, and V987P. In further embodiments, the RNA or DNA comprises, or alternatively consists essentially of, or yet further consists of a polynucleotide encoding SEQ ID NO: 5 or an equivalent thereof.

In some embodiments, the S protein comprises, or alternatively consists essentially of, or yet further consists of the polypeptide of SEQ ID NO: 6 or an equivalent thereof. In some embodiments, the equivalent of SEQ ID NO: 6 retains the mutations of SEQ ID NO: 6, i.e., T19R, T95I, G142D, E156Δ, F157Δ, R158G, L452R, T478K, D614G, R682S, R685G, D950N, K986P, and V987P. In further embodiments, the RNA or DNA comprises, or alternatively consists essentially of, or yet further consists of a polynucleotide encoding SEQ ID NO: 6 or an equivalent thereof.

In some embodiments, the S protein comprises, or alternatively consists essentially of, or yet further consists of the polypeptide of SEQ ID NO: 7 or an equivalent thereof. In some embodiments, the equivalent of SEQ ID NO: 7 retains the mutations of SEQ ID NO: 7, i.e., T19R, T95I, G142D, E156Δ, F157Δ, R158G, L452R, T478K, D614G, P681R, D950N, F817P, A892P, A899P, A942P, K986P, V987P, R682S and R685G. In further embodiments, the RNA comprises, or alternatively consists essentially of, or yet further consists of the polynucleotide of SEQ ID NO: 9 or an equivalent thereof. In some embodiments, the equivalent of SEQ ID NO: 9 still encodes SEQ ID NO: 7 or an equivalent thereof.

In some embodiments, the S protein comprises, or alternatively consists essentially of, or yet further consists of the polypeptide of SEQ ID NO: 10 or an equivalent thereof. In some embodiments, the equivalent of SEQ ID NO: 10 retains the mutations of SEQ ID NO: 10, i.e., T19R, T95I, G142D, E156Δ, F157Δ, R158G, L452R, T478K, D614G, D950N, F817P, A892P, A899P, A942P, K986P, V987P, R682S and R685G. In further embodiments, the RNA or DNA comprises, or alternatively consists essentially of, or yet further consists of a polynucleotide encoding SEQ ID NO: 10 or an equivalent thereof.

In some embodiments, the S protein comprises, or alternatively consists essentially of, or yet further consists of the polypeptide of SEQ ID NO: 11 or an equivalent thereof. In some embodiments, the equivalent of SEQ ID NO: 11 retains the mutations of SEQ ID NO: 11, i.e., T19R, V70F, T95I, G142D, E156Δ, F157Δ, R158G, A222V, W258L, K417N, L452R, T478K, D614G, P681R, D950N, F817P, A892P, A899P, A942P, K986P, V987P, R682S and R685G. In further embodiments, the RNA comprises, or alternatively consists essentially of, or yet further consists of the polynucleotide of SEQ ID NO: 13 or an equivalent thereof. In some embodiments, the equivalent of SEQ ID NO: 13 still encodes SEQ ID NO: 11 or an equivalent thereof.

In some embodiments, the S protein comprises, or alternatively consists essentially of, or yet further consists of the polypeptide of SEQ ID NO: 14 or an equivalent thereof. In some embodiments, the equivalent of SEQ ID NO: 14 retains the mutations of SEQ ID NO: 14, i.e., T19R, V70F, T95I, G142D, E156Δ, F157Δ, R158G, A222V, W258L, K417N, L452R, T478K, D614G, P681R, D950N, E484K, N440K, F817P, A892P, A899P, A942P, K986P, V987P, R682S and R685G. In further embodiments, the RNA comprises, or alternatively consists essentially of, or yet further consists of the polynucleotide of SEQ ID NO: 16 or an equivalent thereof. In some embodiments, the equivalent of SEQ ID NO: 16 still encodes SEQ ID NO: 14 or an equivalent thereof. In one aspect, the RNA comprises, or consists essentially of, or yet further consists of a sequence disclosed in SEQ ID NO: 55, or an equivalent thereof.

In some embodiments, the equivalent of any one of SEQ ID NOs: 9, 13, or 16 consists of an GC content of about 35% to about 70% across the full length of the equivalent.

In some embodiments, the equivalent is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or more identical to the full-length reference sequence.

In some embodiments, the RNA further comprises a 3′ UTR. In further embodiments, the 3′UTR comprises, or alternatively consists essentially of, or consists of any one of SEQ ID NOs: 18, 22, or 24.

In some embodiments, the RNA further comprises a 5′ UTR. In further embodiments, the 5′ UTR comprises, or alternatively consists essentially of, or consists of SEQ ID NO: 20 or 26.

In some embodiments, the RNA further comprises a polyA tail. In further embodiments, the poly A tail comprises any one of SEQ ID NOs: 27, 28, or 30.

In some embodiments, the RNA further comprises a 5′ cap. In further embodiments, the 5′ cap comprises, or alternatively consists of, or yet further consists of a 5′ CleanCap. This structure uses an initiating capped trimer to yield a naturally occurring 5′ cap structure.

In some embodiments, the RNA comprises, or alternatively consists essentially of, or consist of, optionally from 5′ to 3′, a 5′UTR, a coding sequence encoding an S protein or a fragment as disclosed herein, a 3′UTR and a polyA. In further embodiments, the RNA comprises, or alternatively consists essentially of, or consists of SEQ ID NO: 32 or 52.

In some embodiments, the RNA is a messenger RNA (mRNA). Further provided are DNA molecules encoding these RNA as well as the complements thereof.

In some embodiments, the RNA is chemically modified. In further embodiments, the modification comprises, or alternatively consists essentially of, or consists of modifying a uridine (U) residue to an N1-methyl-pseudouridine residue. Additionally or alternatively, the modification comprises, or alternatively consists essentially of, or consist of modifying a U residue to a pseudouridine residue.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of residues of the RNA is chemically modified.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of uridine residues of the RNA is chemically modified, optionally to N1-methyl pseudouridine or pseudouridine. In further embodiments, at least about 50%, or at least about 70%, or about 100% of the uridine residues in the RNA are N1-methyl pseudouridine or pseudouridine.

In some embodiments, all or some of uridine residues are replaced by pseudouridines during in vitro transcription. This modification stabilizes the mRNA against enzymatic degradation in the cell, leading to enhanced translation efficiency of the mRNA. The pseudouridines used can be N1-methyl-pseudouridine, or other modifications that are well known in the art such as N6m-ethyladenosine (m6A), inosine, pseudouridine, 5-methylcytidine (m5C), 5-hydroxymethylcytidine (hm5C), and N1-methyladenosine (m1A). The modification optionally is made throughout the entire mRNA. The skilled artisan will recognize that other modified RNA residues can be used to stabilize the protein's 3 dimensional structure and increase protein translation.

Without wishing to be bound by the theory, an RNA encoding a naturally occurring S protein activates an endosomal RNA sensing pathway such as TLR3, TLR7, and TLR8 (Toll-like receptor), thereby induces innate immunity which in turn inhibits spike protein translation. In addition, a secreted IFN-β provokes tumor cell death upon binding of cognate receptor expressed on the cell surface by activation of the downstream apoptotic pathway. However, an optimized RNA expressing a mutated S protein as disclosed herein avoids this disadvantage, and thus presents an improved translation efficiency (innate immunity) which in turn inhibits spike protein translation. In some embodiments, the optimized RNA can be administered to a subject in need thereof, expressing the mutated S protein in vivo. In further embodiments, the expressed S protein can induce an immune response in the subject, which in turns preventing or treating a SARS-COV-2 infection as disclosed herein. Additionally or alternatively, the optimized RNA expresses the mutated S protein in vitro and optionally such expressed S protein can activate an immune cell in vitro. The activated immune cells can then be used to treat a subject in need thereof.

In another aspect, provided is a method of producing a spike (S) protein or an immunogenic fragment thereof of a severe acute respiratory syndrome coronavirus 2 (SARS-CoV-2). In some embodiments, the method is an in vitro method. The method comprises, or alternatively consists essentially of, or yet further consists of culturing a cell as disclosed herein under conditions suitable for expressing the S protein or immunogenic fragment thereof. In further embodiments, the method herein further comprises isolating the S protein or immunogenic fragment thereof. In some embodiments, the cell is a host cell as disclosed herein.

Additionally, provided is a method for screening a candidate agent reducing or inhibiting the binding of SARS-COV-2 and its receptor, such as ACE2, optionally in a subject or on a cell of the subject or both. The method comprises, or alternatively consist essentially of, or yet further consists of expressing a spike (S) protein or an immunogenic fragment thereof from an RNA as disclosed herein, and measuring the binding between the expressed S protein or immunogenic fragment thereof and the SARS-COV-2 receptor, such as ACE2, with or without the presence of the candidate agent or with different concentrations of the candidate agent. In some embodiments, less binding between the expressed S protein or immunogenic fragment thereof and the SARS-COV-2 receptor with the presence of the candidate agent compared to without the candidate agent, indicates that the candidate agent reduces or inhibits the binding of SARS-COV-2 and its receptor. In some embodiments, decreased binding between the expressed S protein or immunogenic fragment thereof and the SARS-COV-2 receptor while increasing the concentration of the candidate agent indicates the candidate agent reduces or inhibits the binding of SARS-COV-2 and its receptor. In some embodiments, the S protein or immunogenic fragment thereof is expressed in a host cell in the measuring step. Additionally or alternatively, the receptor, such as ACE2, is expressed in a host cell in the measuring step. In some embodiments, the receptor, such as ACE2, is isolated from a host cell in the measuring step, In other embodiments, the S protein or immunogenic fragment thereof is isolated from a host cell in the measuring step. In some embodiments, the isolated S protein or immunogenic fragment thereof or the isolated receptor further comprises a detectable label, such as a fluorescent protein. In further embodiments, the binding between the S protein or immunogenic fragment thereof and the receptor is performed using a fluorescence-based assay, such as fluorescent microscopy or Fluorescence-Activated Cell Sorting (FACS)

In yet a further aspect, provided is a method for selecting an RNA encoding a spike (S) protein or an immunogenic fragment thereof of a severe acute respiratory syndrome coronavirus 2 (SARS-COV-2). The method comprises, or alternatively consists essentially of, or yet further consists of transducing the RNA into a cell, culturing the cell under conditions suitable for expressing the RNA, and measuring IFN-α or IFN-β or both secreted by the cell. In some embodiments, the method further comprises selecting the RNA if no secretion of IFN-α or IFN-β or both or less secretion of IFN-α or IFN-β or both compared to an RNA encoding a wild type S protein or an immunogenic fragment thereof. In some embodiments, the IFN-α or IFN-β or both is measured using enzyme-linked immunosorbent assay.

### Polynucleotides, Vectors, Cells and Related Methods

In one aspect, provided is a polynucleotide encoding an RNA as disclosed herein, or a polynucleotide complementary thereto. In some embodiments, the polynucleotide is selected from the group of: a deoxyribonucleic acid (DNA), an RNA, a hybrid of DNA and RNA, or an analog of each thereof.

In some embodiments, the disclosure provides a polynucleotide encoding a spike (S) protein or an immunogenic fragment thereof of a severe acute respiratory syndrome coronavirus 2 (SARS-COV-2) the S protein or immunogenic fragment thereof comprising at least one non-naturally occurring amino acid mutation. A non-limiting example of such polynucleotide comprises, or alternatively consists essentially of, or consists of a the sequence of SEQ ID NO: 55. In some embodiments, the disclosure provides a polynucleotide encoding a spike (S) protein or an immunogenic fragment thereof of a severe acute respiratory syndrome coronavirus 2 (SARS-COV-2) comprising at least one non-naturally occurring amino acid mutation, wherein the polynucleotide comprises, or alternatively consists essentially of, or consists of a the sequence of SEQ ID NO: 52.

In a further aspect, provided is a vector comprising, or alternatively consisting essentially of, or consisting of a polynucleotide as disclosed herein. In some embodiments, the polynucleotide is a vector comprising, or alternatively consisting essentially of, or consisting of the sequence of SEQ ID NO: 53.

In some embodiments, the vector further comprises a regulatory sequence operatively linked to the polynucleotide to direct the transcription thereof. In some embodiments, the regulatory sequence is suitable for use in an in vitro transcription system. In further embodiments, the regulatory sequence comprises, or alternatively consists essentially of, or consists of a promotor. In yet further embodiments, the promoter is an RNA polymerase promoter, optionally a bacteriophage RNA polymerase promoter. In some embodiment, the promoter comprises, or consists essentially of, or further consists of a T7 promoter, or a SP6 promoter, or a T3 promoter. In some embodiments, the T7 promoter comprises, or consists essentially of, or yet further consists of TAATACGACTCACTATAA (SEQ ID NO: 51). In some embodiments, the regulatory sequence is suitable for use in a cell to expressing an RNA as disclosed herein. In further embodiments, the regulatory sequence comprises, or alternatively consists essentially of, or yet further consists of a promotor, or an enhancer or both.

In some embodiments, the vector further comprises a regulatory sequence operatively linked to the polynucleotide to direct the replication thereof. In further embodiments, the regulatory sequence comprises, or alternatively consists essentially of, or yet further consists of one or more of the following: an origin of replication or a primer annealing site, a promoter, or an enhancer.

In some embodiments, an RNA, or a polynucleotide, or a vector further comprises a marker selected from a detectable marker, a purification marker, or a selection marker.

In some embodiments, the vector is a non-viral vector, optionally a plasmid, or a liposome, or a micelle. In some embodiments, the plasmid comprises, or alternatively consists essentially of, or consists of SEQ ID NO: 33 or an equivalent thereof. In some embodiments, the vector is a viral vector, optionally an adenoviral vector, or an adeno-associated viral vector, or a retroviral vector, or a lentiviral vector, or a plant viral vector.

In some embodiments, a polynucleotide or a vector as disclosed herein is suitable for producing (such as transcribing or expressing or replicating) an RNA as disclosed herein. Such production can be in vivo or in vitro. For example, the polynucleotide or vector can be used to produce or replicate the RNA in vitro. Such RNA is then administrated to a subject in need thereof optionally with a suitable pharmaceutical acceptable carrier. Alternatively, the polynucleotide or vector can be used as a gene therapy and directly administrated to a subject in need thereof optionally with a suitable pharmaceutical acceptable carrier. In further embodiments, the gene therapy can additionally deliver other prophylactic or therapeutic agent to the subject.

In another aspect, a cell comprising one or more of: an RNA as disclosed herein, a polynucleotide as disclosed herein, or a vector as disclosed herein. In some embodiments, the cell is a prokaryotic cell, optionally an Escherichia coli cell. In some embodiments, the cell is a eukaryotic cell, optionally a mammal cell, an insect cell, or a yeast cell. In some embodiments, the cell is a human embryonic kidney 293 cell (HEK 293 cell or 293 cell) or a 293T cell.

In some embodiments, a cell as disclosed herein is suitable for producing (such as transcribing or expressing) an RNA as disclosed herein. Such production can be in vivo or in vitro. For example, the cell can be used to produce the RNA in vitro. Such RNA is then administrated to a subject in need thereof optionally with a suitable pharmaceutical acceptable carrier. Alternatively, the cell can be used as a cell therapy and directly administrated to a subject in need thereof optionally with a suitable pharmaceutical acceptable carrier. In further embodiments, the cell therapy can additionally deliver other prophylactic or therapeutic agent to the subject.

In yet another aspect, provided is a composition comprising, or alternatively consisting essentially of, or yet further consisting of a carrier, optionally a pharmaceutically acceptable carrier, and one or more of: an RNA as disclosed herein, a polynucleotide as disclosed herein, a vector as disclosed herein, or a cell as disclosed herein.

In some embodiments, the composition further comprises an additional prophylactic or therapeutic agent.

In some embodiments, the additional prophylactic or therapeutic agent is suitable for preventing or treating a SARS-COV-2 related disease as disclosed herein. In further embodiments, the additional prophylactic or therapeutic agent comprises, or alternatively consists essentially of, or yet further consists of an anti-viral agent, optionally remdesivir, lopinavir, ritonavir, ivermectin, tamiflu, or favipiravir; an anti-inflammatory agent, optionally dexamethasone, tocilizumab, kevzara, colcrys, hydroxychloroquine, chloroquine, or a kinase inhibitor; a covalescent plasma from a subject recovered from a SARS-COV-2 infection; an antibody binding to SARS-COV-2, optionally bamlanivimab, etesevimab, casirivimab, or imdevimab; or an antibiotic agent, optionally azithromycin.

In some embodiments, the additional prophylactic agent is suitable for preventing a disease that is not related to SARS-COV-2. For example, the additional prophylactic agent comprises, or alternatively consists essentially of, or yet further consists of a vaccine for another coronavirus, such as SARS-COV or MERS-COV. Additionally or alternatively, the additional prophylactic agent comprises, or alternatively consists essentially of, or yet further consists of a vaccine for another virus, such as an influenza (flu) vaccine, a papillomavirus vaccine, an Hepatitis A vaccine, an Hepatitis B vaccine, an Hepatitis c vaccine, a polio vaccine, a chickenpox varicella vaccine, a measles vaccine, a mumps vaccine, a rubella vaccine, a rotavirus vaccine. In some embodiments, the additional prophylactic agent comprises, or alternatively consists essentially of, or yet further consists of a vaccine for a bacterium or other pathogen, such as a diphtheria vaccine, a Haemophilus influenzae type b vaccine, a Pertussis vaccine, a pneumococcus vaccine, a Tetanus vaccine, or a Meningococcal vaccine. In some embodiments, the additional prophylactic agent comprises, or alternatively consists essentially of, or yet further consists of a vaccine for a non-infectious disease, such as a cancer.

In some embodiments, the composition further comprises an adjuvant.

In one aspect, provided is a method of producing an RNA as disclosed herein. In some embodiments, the method comprises, or alternatively consists essentially of, or yet further consists of culturing a cell as disclosure herein under conditions suitable for expressing and/or replicating the RNA. In further embodiments, the RNA is produced by a plasmid DNA (pDNA) vector delivery system. In yet further embodiments, the plasmid vectors can be adapted for mRNA vaccine production. Commonly used plasmids include pSFV1, pcDNA3 and pTK126, which are all commercially available. One unique mRNA expression system is pEVL (see, Grier et al. Mol Ther Nucleic Acids. 19; 5:e306 (2016)).

In some embodiments, the method comprises, or alternatively consists essentially of, or yet further consists of contacting a polynucleotide as disclosed herein or a vector as disclosed herein with an RNA polymerase, adenosine triphosphate (ATP), cytidine triphosphate (CTP), guanosine-5′-triphosphate (GTP), and uridine triphosphate (UTP) or a chemically modified UTP under conditions suitable for expressing the RNA. In some embodiments, the RNA is produced in a linear in vitro transcription (IVT) system from a linear DNA template comprising a bacteriophage promoter, UTRs and a coding sequence, by using a RNA polymerase (T7, T3 or SP6) and a mix of the different nucleosides. In some embodiments, the method further comprises isolating the RNA. In further embodiments, the method further comprises storing the RNA.

### Formulation and Related Methods

In one aspect, provided is a composition comprising, or alternatively consisting essentially of, or yet further consisting of an RNA as disclosed herein and a pharmaceutically acceptable carrier.

In some embodiments, the pharmaceutically acceptable carrier comprises, or alternatively consists essentially of, or yet further consists of a polymeric nanoparticle. In some embodiments, the polymeric nanoparticle comprises, or alternatively consists essentially of, or yet further consists of a Histidine-Lysine co-polymer (HKP). In some embodiments, the HKP comprises a side chain selected from SEQ ID NOs: 34-47. Without wishing to be bound by the theory, RNA in the composition has a higher stability compared to the RNA free of a nanoparticle.

In some embodiments, the pharmaceutically acceptable carrier further comprises a lipid. In further embodiments, the lipid comprises a cationic lipid, optionally ionizable. In yet further embodiments, the cationic lipid comprises Dlin-MC3-DMA (MC3) or dioleoyloxy-3-(trimethylammonio)propane (DOTAP) or both. In some embodiments, the lipid further comprises one or more of: a helper lipid, a cholesterol, or a PEGylated lipid.

In some embodiments, the pharmaceutically acceptable carrier comprises, or alternatively consists essentially of, or yet further consists of a lipid nanoparticle (LNP). In further embodiments, the LNP comprises, or alternatively consists essentially of, or yet further consists of one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof. In yet further embodiments, the LNP further comprises one or more of: a helper lipid, a cholesterol, or a PEGylated lipid.

In some embodiments, the helper lipid comprises, or alternatively consists essentially of, or yet further consists of one or more of: disteroylphosphatidyl choline (DSPC), Dipalmitoylphosphatidylcholine (DPPC), (2R)-3-(Hexadecanoyloxy)-2-{[(9Z)-octadec-9-enoyl]oxy}propyl 2-(trimethylazaniumyl)ethyl phosphate (POPC), or dioleoyl phosphatidylethanolamine (DOPE).

As used herein, the term “RL-007” and RL007″ refers to an ionizable lipid equivalent that is prepared by mixing a final concentration of 6.25 mM of SM-102, 1.25 mM of DSPC, 4.815 mM of Cholesterol, and 0.1875 of mM DMG-PEG2000 (i.e., a 50:10:38:1.5 molar ratio). The Terms “RL-007 vaccine”, “RL007 vaccine”, “RL-007 mRNA vaccine”, or “RL007 mRNA vaccine” refer to a vaccine which comprises, or alternatively consists essentially of, or yet further consists of an RL-007 carrier. In some embodiments, the pharmaceutically acceptable carrier comprises, or alternatively consists essentially of, or yet further consists of RL-007.

In some embodiments, wherein the cholesterol comprises, or alternatively consists essentially of, or yet further consists of a plant cholesterol or an animal cholesterol or both.

In some embodiments, the PEGylated lipid comprises, or alternatively consists essentially of, or yet further consists of one or more of: PEG-c-DOMG (R-3-[(ω-methoxy-poly(ethyleneglycol)2000)carbamoyl)]-1,2-dimyristyloxypropyl-3-amine), PEG-DSG (1,2-Distearoyl-sn-glycerol, methoxypolyethylene glycol), PEG-DMG (1,2-Dimyristoyl-sn-glycerol) optionally PEG2000-DMG ((1,2-dimyristoyl-sn-glycero-3-phophoethanolamine-N-[methoxy(polyethylene glycol)-2000)], or PEG-DPG (1,2-Dipalmitoyl-sn-glycerol, methoxypolyethylene glycol).

In a further aspect, provided is a method of producing a composition comprising, or alternatively consisting essentially of, or yet further consisting of an RNA as disclosed herein and an HKP. The method comprises, or alternatively consists essentially of, or yet further consists of contacting the RNA with an HKP, thereby the RNA and the HKP are self-assembled into nanoparticles.

In some embodiments, the mass ratio of HKP and the RNA in the contacting step is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of HKP and the RNA in the contacting step is about 2.5:1. In another embodiment, the mass ratio of HKP and the RNA in the contacting step is about 4:1.

In some embodiments, the method further comprises contacting the HKP and RNA with a cationic lipid. In further embodiments, the cationic lipid comprises, or consists essentially of, or yet further consists of Dlin-MC3-DMA (MC3) or DOTAP (dioleoyloxy-3-(trimethylammonio)propane) or both. In yet further embodiments, the mass ratio of the cationic lipid and the RNA in the contacting step is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the RNA and the cationic lipid in the contacting step is about 1:1. Accordingly, the mass ratio of the HKP, the RNA and the cationic lipid in the contacting step can be calculated based on the ratio between the HKP and the RNA and the ratio between the RNA and the cationic lipid. For example, if the ratio of the HKP to the RNA is about 4:1 and the ratio of the RNA to the cationic lipid is about 1:1, the ratio of the HKP to the RNA to the cationic lipid is about 4:1:1.

In one embodiments, HKP(+H) stock solution (10 mg/mL) was prepared in nuclease free water. A concentrated stock solution was diluted to 2.5 mg/mL in water. mRNA working solution (1 mg/mL) was prepared in 1 mM citrate buffer (pH 6.0). The mRNA/HKP(+H) polyplex was formed by mixing equal volumes of 2.5 mg/mL HKP(+H) and 1 mg/mL mRNA using microfluidics. The mass ratio of HKP(+H) to mRNA is 2.5:1. The mRNA/HKP(+H) polyplex was incubated for 30 min at room temperature before use. For preparation of all peptide based polyplexes, the size was determined with the Zetasizer (Malvern Panalytical) prior to its transfection or injection.

In some embodiments, the pharmaceutical comprises, or consists essentially of or yet further consists of a polymeric nanoparticle or a lipid nanoparticle both of which comprises a cationic lipid (such as one or more of those as disclosed herein), a helper lipid (such as one or more of those as disclosed herein), a cholesterol (such as one or more of those as disclosed herein) and a PEGylated lipid (such as one or more of those as disclosed herein). In further embodiments, a polymeric nanoparticle further comprises HKP. In some embodiments, the mass ratio of the cationic lipid, helper lipid, cholesterol and PEGylated lipid is about 1:1:1:1:1.

In some embodiments, the mass ratio of the cationic lipid and the helper lipid is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and the helper lipid is about 1:1.

In some embodiments, the mass ratio of the cationic lipid and cholesterol is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and cholesterol is about 1:1.

In some embodiments, the mass ratio of the cationic lipid and PEGylated lipid is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and PEGylated lipid is about 1:1.

The mass ratio of the cationic lipid, helper lipid, cholesterol and PEGylated lipid can be calculated by one of skill in the art based on the ratios of the cationic lipid and the helper lipid, the cationic lipid and the cholesterol and the cationic lipid and the PEGylated lipid as disclosed herein.

In some embodiments, the LNPs comprise, or consists essentially of, or yet further consists of SM-102, DSPC, cholesterol and PEG2000-DMG. In one embodiment, the mass ratio of the SM-102, DSPC, cholesterol and PEG200-DMG is about 1:1:1:1 and/or wherein the molar ratio of the SM-102, DSPC, cholesterol and PEG2000-DMG is about 50:10:38.5:1.5.

In some embodiments, a mass ratio as provided here can be substituted with another parameter, such as a molar ratio, a weight percentage over the total weight, a component's weight over the total volume, or a molar percentage over the total molar amount. Knowing the component and its molecular weight, one of skill in the art would have no difficulty in converting a mass ratio to a molar ratio or other equivalent parameters.

In a further aspect, provided is a method of producing a composition comprising, or alternatively consisting essentially of, or yet further consisting of an RNA as disclosed herein and an LNP. The method comprises, or alternatively consists essentially of, or yet further consists of contacting the RNA with the LNP, thereby the RNA and the LNP are self-assembled into nanoparticles.

In some embodiments, the contacting step is performed in a microfluidic mixer, optionally selected from a slit interdigitial micromixer, or a staggered herringbone micromixer (SHM). In one embodiment, the microfluidic mixer is NanoAssemblr Ignite.

In some embodiments, the composition further comprises an additional prophylactic or therapeutic agent, such as those as disclosed herein. As used herein, the term “prophylactic or therapeutic agent” comprises, consists essentially of, or further consists of a nucleic acid (e.g., an mRNA), compound, polypeptide, antibody, antigen-binding portion thereof, composition, vector, antigen, host cell, and/or any pharmaceutically acceptable compositions comprising antigens, host cells, and/or additional therapeutic agents (e.g., formulations). In some embodiments, the additional prophylactic or therapeutic agent is suitable for preventing or treating a SARS-COV-2 related disease as disclosed herein. In some embodiments, the additional prophylactic or therapeutic agent comprises, or alternatively consists essentially of, or yet further consists of an anti-viral agent, vaccine, or effective dose of a nucleic acid for vaccination, prevention, and treatment against SARS-CoV-2. In some embodiments, the additional prophylactic or therapeutic agent is suitable for preventing or treating a SARS-COV-2 naturally occurring variant, such as a Alpha, Beta, Gamma, Delta, and/or Omicron variant. In some further embodiments, the additional prophylactic or therapeutic agent is suitable for preventing or treating a SARS-CoV-2 naturally occurring variant and its descendent lineages. As used herein, a descendent lineage of a SARS-COV-2 naturally occurring variant is a group of closely related viruses with a common ancestor, all of which cause COVID-19. Descendent lineages of a SARS-CoV-2 naturally occurring variant include, but are not limited to Alpha B.1.1.7 and Q lineages; Beta B.1.351; Gamma P.1; Delta B.1.617.2 and AY lineages; Epsilon B.1.427 and B.1.429; Eta B.1.525; Iota B.1.526; Kappa B.1.617.1; Mu B.1.621, B.1.621.1; Zeta P.2; and/or Omicron B.1.1.529, BA.1, BA.1.1, BA.2, BA.3, BA.4 and BA.5 lineages.

In further embodiments, the additional prophylactic or therapeutic agent comprises, or alternatively consists essentially of, or yet further consists of an anti-viral agent, optionally remdesivir, lopinavir, ritonavir, ivermectin, tamiflu, or favipiravir; an anti-inflammatory agent, optionally dexamethasone, tocilizumab, kevzara, colcrys, hydroxychloroquine, chloroquine, or a kinase inhibitor; a covalescent plasma from a subject recovered from a SARS-COV-2 infection; an antibody binding to SARS-COV-2, optionally bamlanivimab, etesevimab, casirivimab, or imdevimab; or an antibiotic agent, optionally azithromycin.

In some embodiments, the additional prophylactic or therapeutic agent is suitable for preventing a disease that is not related to SARS-COV-2. For example, the additional prophylactic or therapeutic agent comprises, or alternatively consists essentially of, or yet further consists of a vaccine for another coronavirus, such as SARS-COV or MERS-CoV. Additionally or alternatively, the additional prophylactic or therapeutic agent comprises, or alternatively consists essentially of, or yet further consists of a vaccine for another virus, such as an influenza (flu) vaccine, a papillomavirus vaccine, an Hepatitis A vaccine, an Hepatitis B vaccine, an Hepatitis c vaccine, a polio vaccine, a chickenpox varicella vaccine, a measles vaccine, a mumps vaccine, a rubella vaccine, a rotavirus vaccine. In some embodiments, the additional prophylactic or therapeutic agent comprises, or alternatively consists essentially of, or yet further consists of a vaccine for a bacterium or other pathogen, such as a diphtheria vaccine, a Haemophilus influenzae type b vaccine, a Pertussis vaccine, a pneumococcus vaccine, a Tetanus vaccine, or a Meningococcal vaccine. In some embodiments, the additional prophylactic or therapeutic agent comprises, or alternatively consists essentially of, or yet further consists of a vaccine for a non-infectious disease, such as a cancer.

### Methods of Treatment

In one aspect, provided is a method for preventing or treating a disease as disclosed herein. Additionally or alternatively, provided is a method of one or more of: (a) preventing a subject from having a symptomatic SARS-COV-2 infection; (b) preventing a subject from hospitalization after infection by SARS-COV-2; (c) preventing a subject from requiring intensive care (such as in an intensive care unit (ICU)) or a ventilator or both after infection by a SARS-COV; (d) inducing an immune response to SARS-COV-2 in a subject in need thereof; (c) reducing the binding of a SARS-COV-2 or an S protein thereof with its receptor, such as angiotensin converting enzyme 2 (ACE2), in a subject in need thereof; (f) treating a subject infected with SARS-COV-2; or (g) reducing a SARS-COV-2 viral load in a subject in need thereof.

Host neutralizing antibodies block the binding of a SARS-COV-2 or an S protein thereof with its receptors, such as ACE2, resulting in neutralized virus and decreased SARS-COV-2 viral loads. Viral load is a measure of the severity of a viral infection, and can be calculated directly by estimating the amount of virus in an infected organism, an involved body fluid, or a biological sample. Quantification of viral load can generally be performed by utilizing a Plaque Reduction Neutralization Assay (PRNT). PRNT is a serological test which utilizes the ability of a specific antibody to neutralize a virus, in turn, preventing the virus from causing the formation of plaques in a cell monolayer. The assay involves mixing a constant amount of virus with dilutions of the serum specimen being tested, followed by plating of the mixture onto cells of an appropriate cell line for the individual virus. The concentration of plaque forming units can be determined by the number of plaques formed after a few days. A dye is added for visualization of the plaques, and the number of plaques in an individual plate is divided by the original number of virons to calculate the percentage neutralization. Depending on the virus, the plaque forming units (p.f.u.) are measured by microscopic observations, fluorescent antibodies, or specific dyes that react with the infected cell. Neutralizing titers can be calculated as the reciprocal of the lowest dilution that resulted in a greater than 50% reduction (PRNT₅₀) or 90% reduction (PRNT₉₀) in p.f.u. (plaque forming units) relative to negative control sera. Sera collected from two fully vaccinated subjects and serum from unvaccinated subjects can be used as a positive and negative controls, respectively.

An induced immune response in a subject therefore comprises inducing increased neutralizing antibody titers, increased splenocyte Th1-cytokine levels (IFN-γ, IL-2, TNF-α) and Th2-cytokines levels (IL-4, IL-5, IL-13), and/or inducing decreased viral loads. The present disclosure provides a method of inducing an immune response in a subject against SARS-COV-2. In some embodiments, the method comprises administering to the subject an effective dose of an RNA, or polynucleotide, or vector, or cell or composition as disclosed herein, thereby inducing in the subject an immune response specific to a SARS-CoV-2 antigen, wherein neutralizing antibody titers in the subject are increased following vaccination relative to neutralizing antibody titers in an unvaccinated subject. In some embodiments, the neutralizing antibody titers in the subject are increased 1 log to 10 log following vaccination relative to neutralizing antibody titers in an unvaccinated subject. In some embodiments, the neutralizing antibody titers in the subject are increased 1 log, 2 log, 3 log, 4 log, 5 log, or 10 log following vaccination relative to neutralizing antibody titers in an unvaccinated subject.

In other embodiments, the immune response is assessed by measuring splenocytes Th1-cytokine levels (IFN-γ, IL-2, TNF-α) and Th2-cytokine levels (IL-4, IL-5, IL-13) in the subject. In some embodiments, the method comprises administering to the subject an effective dose of an RNA, or polynucleotide, or vector, or cell or composition as disclosed herein, thereby inducing in the subject an immune response specific to a SARS-CoV-2 antigen, wherein splenocytes Th1-cytokine levels (IFN-γ, IL-2, TNF-α) and/or Th2-cytokine levels (IL-4, IL-5, IL-13) in the subject are increased following vaccination relative to neutralizing antibody titers in an unvaccinated subject.

The method comprises, or alternatively consists essentially of, or yet further consists of administering to the subject, optionally an effective amount of, one or more of: an RNA as disclosed herein, a polynucleotide as disclosed herein, a vector as disclosed herein, a cell as disclosed herein, or a composition as disclosed herein.

In some embodiments, the method further comprises treating the subject in need thereof, such as administering to the subject, an additional prophylactic or therapeutic agent.

In some embodiments, the additional prophylactic or therapeutic agent is suitable for preventing or treating a SARS-COV-2 related disease as disclosed herein. In further embodiments, the additional prophylactic or therapeutic agent comprises, or alternatively consists essentially of, or yet further consists of an anti-viral agent, optionally remdesivir, lopinavir, ritonavir, ivermectin, tamiflu, or favipiravir; an anti-inflammatory agent, optionally dexamethasone, tocilizumab, kevzara, colcrys, hydroxychloroquine, chloroquine, or a kinase inhibitor; a covalescent plasma from a subject recovered from a SARS-COV-2 infection; an antibody binding to SARS-COV-2, optionally bamlanivimab, etesevimab, casirivimab, or imdevimab; or an antibiotic agent, optionally azithromycin.

In some embodiments, the additional prophylactic agent is suitable for preventing a disease that is not related to SARS-COV-2. For example, the additional prophylactic agent comprises, or alternatively consists essentially of, or yet further consists of a vaccine for another coronavirus, such as SARS-COV or MERS-COV. Additionally or alternatively, the additional prophylactic agent comprises, or alternatively consists essentially of, or yet further consists of a vaccine for another virus, such as an influenza (flu) vaccine, a papillomavirus vaccine, an Hepatitis A vaccine, an Hepatitis B vaccine, an Hepatitis c vaccine, a polio vaccine, a chickenpox varicella vaccine, a measles vaccine, a mumps vaccine, a rubella vaccine, a rotavirus vaccine. In some embodiments, the additional prophylactic agent comprises, or alternatively consists essentially of, or yet further consists of a vaccine for a bacterium or other pathogen, such as a diphtheria vaccine, a Haemophilus influenzae type b vaccine, a Pertussis vaccine, a pneumococcus vaccine, a Tetanus vaccine, or a Meningococcal vaccine. In some embodiments, the additional prophylactic agent comprises, or alternatively consists essentially of, or yet further consists of a vaccine for a non-infectious disease, such as a cancer.

In some embodiments, the subject does not have a SARS-COV-2 infection when administrated with the RNA or the composition. In some embodiments, a SARS-COV-2 infection can be diagnosed using a conventional method, such as a nucleic acid amplification test (NAATs), an antigen test, or an antibody test. NAATs for SARS-COV-2 specifically identify the RNA (ribonucleic acid) sequences that comprise the genetic material of the virus, including but not limited to reverse transcription polymerase chain reaction (RT-PCR), or an isothermal amplification (such as nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR) or strand displacement amplification (SDA)). Antigen tests are immunoassays that detect the presence of a specific viral antigen, which implies current viral infection. More details are available at www.cdc.gov/coronavirus/2019-ncov/lab/resources/antigen-tests-guidelines.html assessed on Aug. 1, 2021. Antibody or serology tests look for antibodies in blood that fight SARS-CoV-2, and are commonly used to indicate a past infection or a successful vaccination. However, IgM antibody can persist for weeks to months following infection, though its persistence appears to be shorter than IgG; therefore, detection of IgM may suggest relatively recent infection. More details are available at www.cdc.gov/coronavirus/2019-ncov/lab/resources/antibody-tests-guidelines.html assessed on Aug. 1, 2021.

In some embodiments, the subject is at risk of having a disease as disclosed herein, such as SARS-COV-2 infection. In some embodiments, the subject has not been exposed to SARS-COV-2. In some embodiments, the subject is at risk of exposing to SARS-CoV-2.

In some embodiments, the subject is more likely than others to become severely ill after being infected by SARS-COV-2. For example, they can require hospitalization, intensive care, or a ventilator, or die, after the infection. In some embodiments, the subject is over age 65. In some embodiments, the subject is over age 45. In some embodiments, the subject has one or more of the following medical conditions: a cancer, a chronic kidney disease, a chronic lung diseases (such as chronic obstructive pulmonary disease (COPD), asthma (moderate-to-severe), interstitial lung disease, cystic fibrosis, or pulmonary hypertension), dementia or other neurological conditions, diabetes (type 1 or type 2), Down syndrome, a heart condition (such as heart failure, coronary artery disease, cardiomyopathies or hypertension), an HIV infection, an immunocompromised state (weakened immune system), a liver disease, overweight, obesity, pregnancy, a sickle cell disease, thalassemia, smoking (current or former), a solid organ or blood stem cell transplant, stroke or cerebrovascular disease (such as those affecting blood flow to the brain), or a substance use disorder.

In some embodiments, the administrations is by inhalation. In further embodiments, the RNA or the composition is atomized by a nebulizer inhalation system prior to or during administration. In yet further embodiments, the nebulizer system is a portable nebulizer for whole respiratory tract drug delivery.

In some embodiments, the administration is by subcutaneous injection. In some embodiments, the administration is by intramuscular injection. In some embodiments, the administration is by intraperitoneal injection (i.p).

In some embodiment, a composition as disclosed herein can be in the form of an aerosol, dispersion, solution, or suspension and can be formulated for inhalation, intramuscular, oral, sublingual, buccal, parenteral, nasal, subcutaneous, intradermal, or topical administration. The term parenteral as used herein includes percutaneous, subcutaneous, intravascular (e.g., intravenous), intramuscular, or intrathecal injection or infusion techniques and the like.

As used herein, an effective dose of an RNA, or polynucleotide, or vector, or cell or composition as disclosed herein is the dose required to produce a protective immune response in the subject to be administered. A protective immune response in the present context is one that prevents or ameliorates disease in a subject challenged with SARS-COV-2 or a pseudovirus thereof. The RNA, or polynucleotide, or vector, or cell or composition as disclosed herein can be administered one or more times. An initial measurement of an immune response to the vaccine may be made by measuring production of antibodies in the subject receiving the RNA, or polynucleotide, or vector, or cell, or composition. Methods of measuring antibody production in this manner are also well known in the art, is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the formulated composition. In some embodiments, an effective dose of an RNA, or polynucleotide, or vector, or cell or composition as disclosed herein is administered twice. In some embodiments, an effective dose of an RNA, or polynucleotide, or vector, or cell or composition as disclosed herein is administered twice at an interval of at least 21 days, at least 28 days, at least 35 days, at least 42 days, at least 49 days, at least 56 days, or at least 64 days

In a further aspect, provided is an inhalation system comprising, or alternatively consisting essentially of, or yet further consisting of an RNA as disclosed herein, a polynucleotide as disclosed herein, a vector as disclosed herein, or a composition as disclosed herein and a nebulizer. In further embodiments, the nebulizer is a portable nebulizer for whole respiratory tract drug delivery.

In some embodiments, the RNA compositions can be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic or prophylactic effect. The desired dosage can be delivered three times a day, two times a day, once a day, every other day, every third day, every week, every two weeks, every three weeks, every four weeks, every 2 months, every three months, every 6 months, etc. In certain embodiments, the desired dosage can be delivered using multiple administrations (e.g., two, three, four, five, six, seven, eight, nine, ten, eleven, twelve, thirteen, fourteen, or more administrations). When multiple administrations are employed, split dosing regimens such as those described herein can be used. In some embodiments, the RNA compositions can be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the RNA compositions can be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the RNA compositions can be administered at an effective dosage level. The effective dose of the RNA, as provided herein, may range from about 10 μg-500 pg, administered as a single dose or as multiple (e.g., booster) doses. In some embodiments, a single dose of a vaccine composition (e.g., administered once, twice, three times, or more) comprises about 10 μg RNA. In some embodiments, a single dose of a vaccine composition (e.g., administered once, twice, three times, or more) comprises about 20 μg RNA. In some embodiments, a single dose of a vaccine composition (e.g., administered once, twice, three times, or more) comprises about 30 μg RNA. In some embodiments, a single dose of a vaccine composition (e.g., administered once, twice, three times, or more) comprises about 40 μg RNA. In some embodiments, a single dose of a vaccine composition (e.g., administered once, twice, three times, or more) comprises about 50 μg RNA. In some embodiments, a single dose of a vaccine composition (e.g., administered once, twice, three times, or more) comprises about 100 μg RNA. In some embodiments, a single dose of a vaccine composition (e.g., administered once, twice, three times, or more) comprises at least 25 pg RNA. In some embodiments, a single dose of a vaccine composition (e.g., administered once, twice, three times, or more) comprises less than 100 μg RNA. In some embodiments, a single dose of a vaccine composition (e.g., administered once, twice, three times, or more) comprises 100 μg or less RNA. In some embodiments, a single dose of a vaccine composition (e.g., administered once, twice, three times, or more) comprises about 250 μg RNA.

In some embodiments, a total amount of RNA administered to a subject is about 10 μg, about 20 μg, about 30 μg, about 40 μg, about 50 μg, about 100 μg, about 200 μg, about 250 μg, or about 500 μg mRNA. In some embodiments, a total amount of RNA administered to a subject is about 50 μg. In some embodiments, a total amount of RNA administered to a subject is about 100 μg. In some embodiments, a total amount of RNA administered to a subject is about 250 μg. In some embodiments, a total amount of RNA administered to a subject is about 500 μg.

In some embodiments, the RNA compositions can be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a the RNA composition can be administered three or four times.

### Kits

In one aspect, provided is a kit for use in a method as disclosed herein.

In some embodiments, the kit comprises, or alternatively consists essentially of, or yet further consist of instructions for use and one or more of: a RNA as disclosed herein, a polynucleotide as disclosed herein, a vector as disclosed herein, a composition as disclosed herein, or an inhalation system as disclosed herein. In further embodiments, the kit is suitable for use in a method of treatment as disclosed herein.

In some embodiments, the kit comprises, or alternatively consists essentially of, or yet further consist of instructions for use and one or more of: an RNA as disclosed herein, a polynucleotide as disclosed herein, a vector as disclosed herein, a cell as disclosed herein, a composition as disclosed herein, an HKP, or a lipid optionally a cationic lipid. In further embodiments, the kit is suitable for use in a method producing an RNA or a composition as disclosed herein.

In some embodiments, the kit comprises, or alternatively consists essentially of, or yet further consist of instructions of use, a polynucleotide or a vector as disclosed herein, an RNA polymerase, ATP, CTP, GTP, and UTP or a chemically modified UTP. In further embodiments, the kit is suitable for use in an in vitro method producing an RNA or a composition as disclosed herein.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: RNA Synthesis

A tested RNA as disclosed herein was synthesized, for example by in vitro transcription (IVT) using a vector as disclosed herein and an IVT kit available to one of skill in the art (such as MAXIscript™ T7 Transcription Kit from ThermoFisher Scientific), and then purified by selective binding of dsRNA to cellulose in an ethanol-containing chromatography buffer containing 10 mM HEPES (pH 7.2), 0.1 mM EDTA, 125 mM NaCl, 16% ethanol and cellulose fibers and centrifugation. Almost 90% of dsRNA could be removed after this procedure. See, for example, Baiersdorfer et. al, 2019, Mol Ther Nucleic Acids. 2019 Apr. 15; 15:26-35. Contaminants could be also eliminated using FPLC and HPLC, see, for example, Kariko et. al, 2011, Nucleic Acids Res. 2011 November; 39(21):e142.

### Example 2: Peptides (HK Polymers) Preparation

The HK peptide polymers were synthesized on a Rainin Voyager synthesizer (Tucson, AZ) by the biopolymer core facility at the University of Maryland.

### Example 3: In Vitro Formulation Preparation

PNI-Genvoy lipid nanoparticle (LNP) formulation: Lipid nanoparticles were formulated using the GenVoy Platform with PNI NanoAssemblr (Precision NanoSystems, Vancouver, British Columbia, Canada) as the positive control in both in vitro and in vivo assays.

HKP(+H) formulation version 1: HKP(+H) stock solution (10 mg/mL) was prepared in nuclease free water. A concentrated stock solution was diluted to 2.5 mg/mL in water. mRNA working solution (1 mg/mL) was prepared in 1 mM citrate buffer (pH 6.0). The mRNA/HKP(+H) polyplex was formed by mixing equal volumes of 2.5 mg/mL HKP(+H) and 1 mg/mL mRNA using microfluidics. The mass ratio of HKP(+H) to mRNA is 2.5:1. The mRNA/HKP(+H) polyplex was incubated for 30 min at room temperature before use. For preparation of all peptide based polyplexes, the size was determined with the Zetasizer (Malvern Panalytical) prior to its transfection or injection.

HKP(+H) formulation version 2: HKP(+H) stock solution (10 mg/mL) was prepared in nuclease free water. A concentrated stock solution was diluted to 4 mg/mL in water. mRNA working solution (1 mg/mL) was prepared in 1 mM citrate buffer (pH 6.0). The mRNA/HKP(+H) polyplex was formed by mixing equal volumes of 4 mg/mL HKP(+H) and 1 mg/mL mRNA. The mass ratio of HKP(+H) to mRNA is 4:1. The mRNA/HKP(+H) polyplex was incubated for 30 min at room temperature before use. The size of each peptide-based polyplex was determined with the Zetasizer (Malvern Panalytical) prior to transfection or injection.

HKP(+H)/DOTAP formulation (post-mixed DOTAP): HKP(+H) stock solution (10 mg/mL) was prepared in nuclease free water. A concentrated stock solution was diluted to 4 mg/mL in water. DOTAP (Sigma-Aldrich) is 1 mg/mL in aqueous buffered solution. mRNA working solution (1 mg/mL) was prepared in 1 mM citrate buffer (pH 6.0). First the mRNA/HKP(+H) polyplex was formed by mixing equal volumes of 4 mg/mL HKP(+H) and 1 mg/mL mRNA. The mRNA/HKP(+H) polyplex was incubated for 30 min at room temperature. Next, the same volume of DOTAP to HKP(+H) solution was added to the mRNA/HKP(+H) polyplex. The mass ratio of HKP(+H)/DOTAP to mRNA was 4:1:1. The mRNA/HKP(+H)/DOTAP nanoparticle was incubated for 30 min at room temperature before use.

HKP(+H)/MC3 or HKP(+H)/DOTAP formulations (pre-mixed MC3 or DOTAP): HKP(+H) stock solution (10 mg/mL) was prepared in nuclease-free water. A concentrated stock solution was diluted to 4 mg/mL in water. DOTAP or MC3 is 1 mg/mL in aqueous buffered solution. mRNA working solution (1 mg/mL) was prepared in 1 mM citrate buffer (pH 6.0). Equal volumes of HKP(+H) and MC3 were pre-mixed at a 4:1 mass ratio and the same volume of mRNA to HKP(+H) solution was added to a pre-mixed HKP(+H)/MC3. The mRNA/HKP(+H)/MC3 nanoparticle was formed by mixing pre-mixed 4 mg/mL HKP(+H)/1 mg/mL MC3 and 1 mg/mL mRNA. The mass ratio of HKP(+H)/MC3 to mRNA is 4:1:1. The mRNA/HKP(+H)/MC3 nanoparticle was incubated for 30 min at room temperature before use.

HKP(+H)/PLA NP formulation: HKP(+H) stock solution (10 mg/mL) was prepared in nuclease free water. A concentrated stock solution was diluted to 4 mg/mL in water. A poly-L-Lactic Acid (PLA) nanoparticle (5 mg/mL) was prepared in water. mRNA working solution (1 mg/mL) was prepared in 1 mM citrate buffer (pH 6.0). Equal volumes of HKP(+H) and mRNA were mixed at a 4:1 mass ratio. The mRNA/HKP(+H) polyplex was incubated for 30 min at room temperature, and then the same volume of PLA nanoparticle to HKP(+H) solution was added to the mRNA/HKP(+H) nanoparticle so that the mRNA/HKP(+H) polyplex was adsorbed on the surface of the PLA nanoparticle. The mass ratio of HKP(+H)/PLA to mRNA was 4:5:1. The mRNA/HKP(+H)/PLA nanoparticle was incubated for 30 min at room temperature before use.

TABLE 6
Lipids and RNA working solutions were prepared according to the Table below.

| | | | Final | 4x | |
|---|---|---|---|---|---|
| | | | working | Stock(mM | |
| | | | solution(12.5 | each) in | |
| | Molar | | mM | 100% | Supplier and Product |
| | ratio | M.W. | lipid) | Ethanol | Information |
| Ionizable | 50 | | 6.25 | 25.00 | |
| lipid | | | | | |
| equivalent | | | | | |
| (e.g., | | | | | |
| SM-102) | | | | | |
| DSPC | 10 | 790.145 | 1.25 | 5.00 | avantilipids.com/product/850365 |
| | | | | | (last accessed on Aug. 9th, 2021) |
| Cholesterol | 38.5 | 386.654 | 4.8125 | 19.25 | avantilipids.com/product/700100 |
| | | | | | (last accessed on Aug. 9th, 2021) |
| PEG2000- | 1.5 | 2509.2 | 0.1875 | 0.75 | broadpharm.com/web/product.php?catalog=BP- |
| DMG | | | | | 25496 |
| | | | | | (last accessed on Aug. 9th, 2021) |
| mRNA | TriLink | | in 50 mM | | www.trilinkbiotech.com/custom- |
| | Clean | | Sodium | | mrna-synthesis |
| | Cap | | Citrate | | (last accessed on Aug. 9th, 2021) |
| | mRNA | | | | |

Briefly, the working area was cleaned thoroughly with 70% ethanol. A 4× stock solution of each lipids was made in 100% Ethanol and stored at −20° C. until use. Lipid working solution was prepared by combining each of the following components in a ratio of 1:1:1:1 (final concentration 12.5 mM): Ionizable cationic lipid ((heptadecan-9-yl 8-((2-hydroxyethyl) (6-oxo-6-(undecyloxy) hexyl) amino) octanoate}) SM-102 equivalent, a helper lipid (such as 1,2-Distearoyl-sn-glycero-3 phosphocholine (DSPC)), a cholesterol, and PEG2000-DMG (i.e., 1-monomethoxypolyethyleneglycol-2,3-dimyristylglycerol with polyethylene glycol of average molecular weight 2000). The RNA working solution was prepared in Formulation Buffer (50 mM Sodium Citrate pH4.0). RNA concentration depends on Lipid concentrations, the Flow Rate Ratio and the N/P ratio.

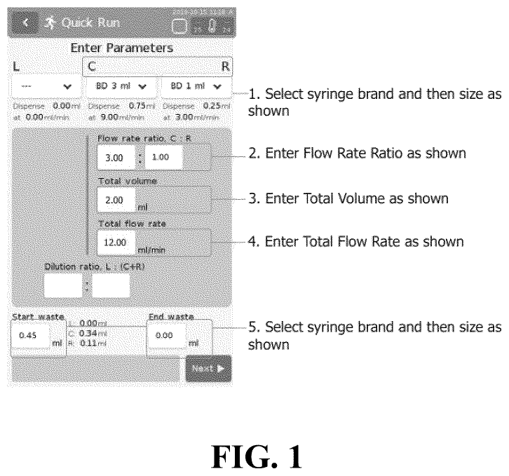

The RNA and lipid nanoparticle was prepared: The NanoAssemblr Ignite was turned on and “Quick Run” was selected from the Main Menu. The parameters were set as shown in FIG. 1 by selecting a field, selecting a value from the drop-down menu or entering the number with the on-screen keyboard and then tapping the check mark. The lid of the Ignite was open and the Cartridge Adaptor was ensured as installed over the “L” inlet of the cartridge slot with the arrow facing upwards, then a NxGen™ cartridge was removed from the package and inserted in the cartridge slot. The rotating block was raised until the cartridge luers were visible. At least 1.5 mL prepared RNA working solution was drawn into a 3 mL syringe. A blunt needle was used if necessary. The needle was removed, air bubbles were cleared from the syringe and the plunger was used to advance the liquid to the tip. Drips from the syringe tip were avoided. The syringe was inserted into the “C” inlet of the Ignite Cartridge and twisted clockwise to engage the Luer Lock. At least 0.5 mL prepared Lipid Working solution was drawn into a 1 mL syringe using a blunt needle if necessary. The needle was removed, air bubbles were cleared from the syringe and the plunger was used to advance the liquid to the tip. Drips from the syringe tip were avoided. The syringe was inserted into the “R” inlet of the Ignite Cartridge. The rotating block was returned to the downwards position. The sample switch arm for two 15 mL conical tubes was ensured as installed. A 15 mL conical collection tube was marked with “RNA-LNP” and pushed into the clip labeled “Sample”. Another tube was marked with “waste” and pushed into the clip labeled “Waste”. The Ignite lid was closed and “Next” on the screen was tapped. The parameters and the information in the dialog box were confirmed. The “Start” button was pressed. The pushers of the Ignite™ were then injecting the fluids into the microfluidic cartridge. The formulation was collected in the tube labeled “RNA-LNP”. After the motors were positioned themselves back in the home position, the screen indicated when it was safe to open the lid. The lid was open once it was safe and the conical collection tube labeled “RNA-LNP” was removed and set aside for characterization and further processing immediately. The rotating block was raised and the syringes were removed from Ignite™ and discarded. The rotating block was returned to the downward position and the NxGen cartridge was removed and discarded. To make additional samples, the back “<” button was tapped to return to the Quick Run Screen and the steps described in this paragraph were repeated except the initial quick run setting step.

For the characterization of formulated lipid nanoparticles (LNPs), following preparation, 25-50 μL of the sample fraction was mixed with 650 μL of ultra pure water (Invitrogen) and the intensity-averaged particle size (Z-average) was measured on ZetaSizer (Malvern Instruments Inc.).

The sample fraction was transferred immediately to a Slide-a-lyzer G2 dialysis cassette (10000 MWCO, Thermo Fischer Scientific Inc.) and dialyzed over night at 4° C. against PBS (pH7.4).

The LNP formulations were concentrated using Amicon ultra-centrifugal filters (EMD Millipore, Billerica, MA, USA), passed through a 0.22-μm filter (Acrodisc) and stored at 4° C. (PBS).

The sample fraction was also collected and measured for the particle size (post dialysis particle size).

The final mRNA concentration and encapsulation efficiency (EE) were measured using Quant-it Ribogreen Assay Kit (Thermo Fischer Scientific).

### Example 4: In-Vitro Transfection of mRNA

To verify the proper protein expression of RNAs, an EGFP mRNA or a tested RNA as disclosed herein is transiently transfected into human embryonic kidney 293T cells (293T cells). Briefly, 4.8×10⁵ cells are plated into a 6 well plate containing 2 ml of DMEM (10% fetal bovine serum and 1% Penicillin-Streptomycin (ThermoFisher Scientific)). After 24 hr, when the cells are 70-90% confluent, the EGFR mRNA or the tested RNA is transfected into 293T cells using Lipofectamine MessengerMAX Transfection Reagent (ThermoFisher Scientific) according to the manual protocol. The transfected 293T cells are cultured for two day and then measured for in vitro protein expression.

Various formulations/carriers as disclosed herein are also examined for their ability to carry the EGFP mRNA or the tested RNA into a target host cell, such as human embryonic kidney 293 cells (HEK 293 cells). Briefly, 4.8×10⁵ cells are plated into a 6 well plate containing 2 ml of DMEM (10% fetal bovine serum and Penicillin-Streptomycin (ThermoFisher Scientific)). After 24 hr, when the cells are 70-90% confluent, a formulation/carrier with the EGFP mRNA or the tested RNA as disclosed herein is added into each well. 293T cells are cultured for two day and then measured for in vitro protein expression.

### Example 5: In Vitro Protein Expression Measurement

Immunofluorescence analysis: Two days post transfection, protein expression is measured by immunofluorescence imaging using a Cytation5 Cell Imaging Multi-Mode Reader (Biotek, Winooski, VT).

Cell lysate preparation: Two days post transfection, culture media is aspirated and cells are washed on ice with ice-cold PBS. Ice-cold lysis buffer (RIPA, ThermoFisher Scientific) with protease inhibitor (ThermoFisher Scientific) is added and the cells are incubated for 30 minutes at 4□C. Cells are then harvested using a cell scraper and lysed by sonication. Centrifugation at 10,000 g for 20 minutes at 4° C. pellets cell debris, and the supernatant is transferred to a fresh microcentrifuge tube. The protein concentration of the lysate is determined by Bradford or BCA protein assay for Western blot.

Western blot: Briefly, in each well of a gel, 20-50 μg of protein is mixed with 4×SDS sample buffer (ThermoFisher Scientific), 10× Reducing buffer (ThermoFisher Scientific), and additional ddH₂O (ThermoFisher Scientific) with a total loading volume of 25 μl/well. The mixture is denatured by heating at 95° C. for 5 minutes and cooled to room temperature and centrifuged before loading onto a NUPAGE™ 4 to 12%, Bis-Tris gel (ThermoFisher Scientific). After electrophoretic separation, the gel is removed from the cassette and transferred using an IBLOT™ 2 Dry Blotting System (ThermoFisher Scientific). The transferred membrane is blocked with 5% fat-free milk powder in TBST for 1 hr at room temperature (RT), incubated with primary antibody for overnight at 4° C., washed three times with TBST (0.05% Tween20 in TBS) buffer, and incubated with secondary antibody, which is HRP conjugated Mouse IgG (H+L) Secondary Antibody (ThermoFisher Scientific, A24512) for 1 hr at RT. Transferred membrane is then developed by Pierce ECL Western Blotting Substrate (ThermoFisher Scientific) and is imaged using chemiluminescent imaging system.

In some embodiments, the primary antibody specifically recognizes and binds to an S protein of SARS-COV-2 or a fragment thereof, such as an S2 protein or a RBD of the S protein or both. Accordingly, expression of a tested RNA to its protein product is assessed. In further embodiments, a loading control for the Western blot was performed. For example, the same sample was tested using Western blot as described above, except the primary antibody specifically recognizes and binds to β-actin. A substantially similar protein levels among groups are found in the loading control.

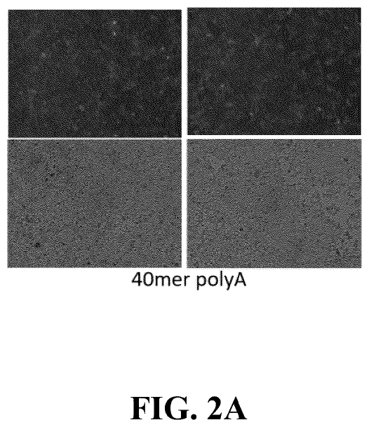

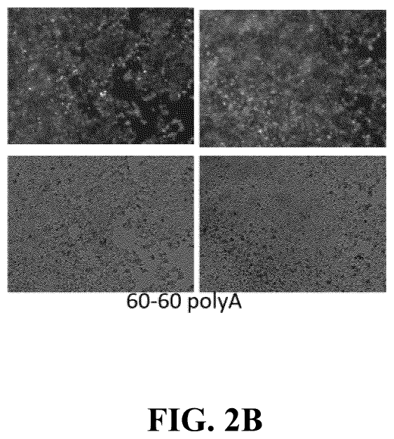

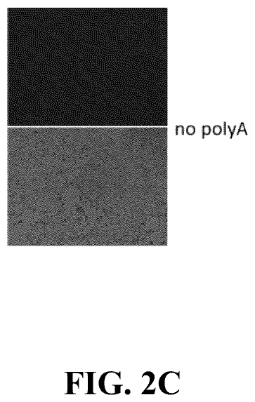

RNAs with different polyA tails, such as a 40 mer polyA (referred to herein as “polyA 40”) (SEQ ID NO: 27) or a 60-60 polyA (see “polyA 60”) (SEQ ID NO: 28), were synthesized, purified, transfected to cells, and tested for protein expression. Briefly, DNA constructs comprising β-globulin 5′ and 3′ UTRs, Blue Fluorescent Protein 2 (BFP2) coding sequence, and different synthetic polyA tails were synthesized by Twist Bioscience. In further embodiments, the DNA constructs further comprise a plasmid backbone comprising, or consisting essentially of, or yet further consisting of a kanamycin selection marker, the pUC57 backbone, and a T7 promoter to make the plasmid in vitro transcription (IVT) competent. IVT was performed as described previously. RNA was transfected into HEK293T cells and Blue Fluorescent Protein (BFP) florescence intensity was observed using a Citation 5 microscope from BioTek. The results are shown in FIG. 2. A top panel of each of FIGS. 2A-2C provides a representative image showing the BFP fluorescence, while a bottom panel of each of FIGS. 2A-2C provides the corresponding bright filed image.

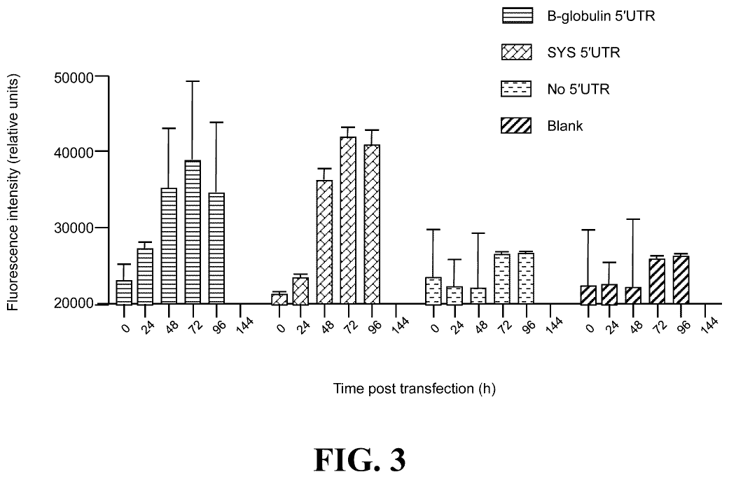

Additionally, RNAs with various UTRs, such as a β-globulin UTR or a SYS UTR as disclosed herein, were synthesized, purified, transfected to cells, and tested for protein expression. Briefly, DNA constructs comprising different 5′UTRs and BFP2 were synthesized by Twist Bioscience. The constructs further comprise a plasmid backbone comprising, or consisting essentially of, or yet further consisting of a kanamycin selection marker, the pUC57 backbone, and a T7 promoter to make the plasmid in vitro transcription (IVT) competent. Once plasmids were prepared and purified, IVT was performed as described previously. RNA was transfected into HEK293T cells using MessengerMax transfection reagent (Thermofisher) and BFP florescent intensity was measured using a Citation 5 microscope from BioTek. Different 5′UTRs were compared at various time points. Sec, FIG. 3. Error bars indicate the standard deviation of 3 replicates.

### Example 6: Binding with Human ACE2

Further investigated is binding between an S protein, a variant and/or a mutant thereof expressed by a tested RNA as disclosed herein and its receptor on a human cell, such as human ACE2. For example, cells (such as HEK293T (Cat #CRL-3216, ATCC) or a549 (Cat #CCL-185, ATCC)) transfected with the tested RNA and expressing the S protein, variant and/or mutant thereof are incubated with a human ACE2 directly or indirectly labeled with a fluorescent protein, such as FITC. The incubated cells are optionally washed to remove the unbound ACE2. Flow cytometry is performed and the mean fluorescence intensity (MFI) of each cell was measured qualitatively. The higher the MFI is, the more ACE2 binds to the cell, indicating a stronger binding (such as showing a higher binding affinity) between ACE2 and the S protein, variant and/or mutant thereof expressed by the cell.

### Example 7: In Vivo Animal Model and Injection

An in vivo study was performed. Briefly, 6-8 week old female BALB/C are randomized into each group, 4 mice per each group and injected intramuscularly into the right flank with 30 μg of an EGFP mRNA with different formulations. With the same formulations mentioned above, a tested RNA is also prepared for in vivo analysis and antibody titer measurement and binding.

On day 28, a second injection for boosting is made and on day 35 serum is collected and analyzed by immunoassay (ELISA) for measurement of antibody titer.

### Example 8: In Vivo Animal Models

Other suitable animal models can be used to investigate a tested RNA in a formulation/carrier as disclosed herein and assess its efficiency for use in a method as disclosed herein and for therapy alone or in combination with other possible therapies, such as anti-inflammatory therapies.

In one example, used herein is a genetically modified animal model expressing human ACE2 optionally under a tissue-specific promoter (for example, the Krt 18 promoter for epithelial cells; K18-hACE2 mice), or a universal promoter (cytomegalovirus enhancer followed by the chicken β-actin promoter) or the endogenous mouse Ace2 promoter. All of these mice are susceptible to infection by SARS-COV-2, but differences in their expression of human ACE2 result in a pathogenic range of mild to lethal disease. In some embodiments, the animal is mouse. Alternative animal models include syrian hamsters, whose ACE2 is significantly similar to human ones and considered as susceptible to infection with SARS-CoV-2; ferrets; and non-human primates.

A RNA composition, such as a tested RNA in a formulation/carrier as disclosed herein, was administered to the animal prior to or concurrently with a challenge with SARS-COV-2 or a pseudovirus thereof. The administration of the RNA composition was repeated at least once or twice. A second administration of the RNA composition occurred about two or about three weeks apart. Animals not challenged with the SARS-COV-2 or pseudovirus served as a negative control while those challenged with the SARS-COV-2 or pseudovirus but not treated served as a positive control. Additional controls were used, such as animals treated with a SARS-COV-2 vaccine as known in the art, such as BNT162b2 available from Pfizer-BioNTech, mRNA-1273 available from Moderna, or JNJ-78436735 from Johnson & Johnson's Janssen, and challenged with the SARS-COV-2 or pseudovirus.

Viral load, lung pathology, immune cell infiltration to the lung, cytokine release, body weight, fur, posture, respiratory distress (such as laboured breathing), lethargy or not, nasal discharge, wheezing, oropharyngeal build-up of mucus, sneezing, loose stools and etc. were monitored after the administration of the RNA composition in order to assess the effects.

### Example 9: Confirmation of Type-I Interferon Secretion (IFN-α and IFN-β) from the A549 Cells Transfected with a Modified or Unmodified SARS-COV-2 Spike mRNA Vaccine Candidates

Experiments were performed to test whether the viral RNA sensing pathway activates the innate immunity in A549 cells transfected with SARS-COV-2 spike mRNA (SBI), results in generation of IFN-β and subsequently repression of SARS-COV-2 S protein translation. The same experimental setting was applied to an RNA expressing a SARS-COV-2 S protein (wild type or comprising S2P or S6P mutations)s, such as an RNA encoding a wild-type SARS-COV-2 S protein (labeled as “SARS-COV-2 Spike WT(SBI)”), an RNA encoding a wild-type SARS-COV-2 S protein with all uridine residues of the RNA chemically modified, optionally to pseudouridine, further optionally to N1-methyl pseudouridine (labeled as “SARS-COV-2 Spike WT(100% modified)”), an RNA encoding a SARS-COV-2 S protein which is a wild type mutated at K986P and V987P with at least one uridine residues of the RNA chemically modified, optionally to pseudouridine, further optionally to N1-methyl pseudouridine (labeled as “SARS-COV-2 Spike S2P WT”), an RNA encoding a SARS-COV-2 S protein which is a wild type mutated at D614G, K986P and V987P with at least one uridine residues of the RNA chemically modified, optionally to pseudouridine, further optionally to N1-methyl pseudouridine (labeled as “SARS-COV-2 Spike S2P D614G”), an RNA encoding a SARS-COV-2 S protein which is a wild type mutated at D614G with at least one uridine residues of the RNA chemically modified, optionally to pseudouridine, further optionally to N1-methyl pseudouridine (labeled as “SARS-COV-2 Spike D614G WT”), a sequence optimized RNA encoding a SARS-COV-2 S protein which is a wild type mutated at F817P, A892P, A899P, A942P, K986P, and V987P with at least one uridine residues of the RNA chemically modified, optionally to pseudouridine, further optionally to N1-methyl pseudouridine (labeled as “SARS-COV-2 Spike S6P (RNAimmune)”), an RNA encoding a beta variant of SARS-COV-2 S protein firstly identified in South Africa and further mutated at F817P, A892P, A899P, A942P, K986P, and V987P with at least one uridine residues of the RNA chemically modified, optionally to pseudouridine, further optionally to N1-methyl pseudouridine (labeled as “SARS-COV-2 Spike S6P_SA”), a sequence optimized RNA encoding a beta variant of SARS-COV-2 S protein firstly identified in South Africa and further mutated at F817P, A892P, A899P, A942P, K986P, and V987P with at least one uridine residues of the RNA chemically modified, optionally to pseudouridine, further optionally to N1-methyl pseudouridine (labeled as “SARS-COV-2 Spike S6P_SA_GS”), or an RNA encoding a RBD of a SARS-COV-2 S protein with at least one uridine residues of the RNA chemically modified, optionally to pseudouridine, further optionally to N1-methyl pseudouridine (labeled as “RBD”). See more details in the table below. In further embodiments, the wild type SARS-COV-2 S protein comprises, or consists essentially of, or yet further consists of a polypeptide as set forth in SEQ ID NO: 1.

TABLE 7

| | | Sequence | Modified |
|---|---|---|---|
| Sequence | Description | Optimized? | Uridine? |
| SARS-CoV-2 Spike | wild type spike protein | No | No |
| WT(SBI) | | | |
| SARS-CoV-2 Spike | wild type spike protein | No | Yes |
| WT(100% modified) | with 100% uridine | | |
| | modification | | |
| SARS-CoV-2 Spike | wild type spike with | No | Yes |
| S2P WT | S2P mutation | | |
| SARS-CoV-2 Spike | wild type spike with | No | Yes |
| S2P D614G | S2P mutation and | | |
| | D614G mutation | | |
| SARS-CoV-2 Spike | wild type spike with | No | Yes |
| D614G WT | D614G mutation | | |
| SARS-CoV-2 Spike | Spike with S6P | Yes | Yes |
| S6P (RNAimmune) | mutation | | |
| SARS-CoV-2 Spike | Spike with S6P and | No | Yes |
| S6P_SA | South Africa mutation | | |
| SARS-CoV-2 Spike | | Yes | Yes |
| S6P_SA_GS | | | |
| RBD | RBD region only | No | Yes |

Additionally, the same experimental setting is applied to a tested RNA in a formulation/carrier as disclosed herein.

### Materials and Methods

293T cells were transiently transfected with RNAs encoding one of the following and formulated at an about 1:3 ratio of RNA to lipid:SARS-COV-2 Spike WT(SBI), SARS-COV-2 Spike WT(100% modified), SARS-COV-2 Spike S2P WT, SARS-CoV-2 Spike S2P D614G, SARS-COV-2 Spike D614G WT, SARS-COV-2 Spike S6P (RNAimmune), SARS-COV-2 Spike S6P_SA, SARS-COV-2 Spike S6P_SA_GS, or RBD.

2 ml of A549 cells were plated at a density of 2.5×10⁵ cells in 12 well plates. The cells were incubated for 24 hours at 37° C. in a 5% CO₂ incubator. RNA(lug) was diluted in 50 μl of Opti-MEM-I. 7.5 μl of Lipofectamine MessengerMAX (Invitrogen, Cat #LMRNA008) was also diluted and incubated for 10 minutes at RT. The diluted mRNA mixture was added to the diluted lipid. Incubation was performed for 5 minutes at RT. mRNA-lipid complex was added to the cells and incubated for 24 hours at 37° C. in a 5% CO₂ incubator.

ELISA detecting IFN-α was performed using VeriKine Human IFN-α ELISA Kit (Cat 41100, pbl assay science). 1 ml of the culture supernatant was transferred to a new tube and centrifuged for 5 minutes at 3000 rpm. The culture supernatant was then transferred to a new tube and stored at −80° C. until use.

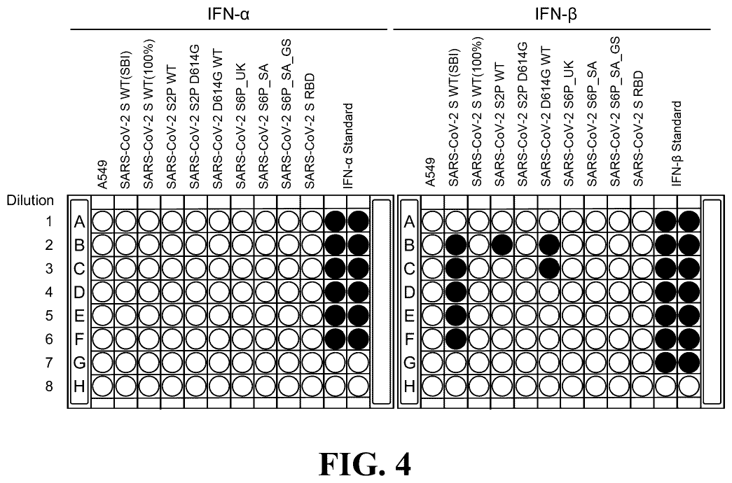

A standard curve was generated: a high sensitivity standard curve 12.5-500 pg/ml or extended range standard curve 156-5000 pg/ml was constructed. Six polypropylene tubes (S1-S6) were labeled and filled with 250 μl Dilution Buffer. Polypropylene tips were used to add the Human IFN-α Standard to S6 and mix gently. Tips were changed between each dilution. 250 μl was removed from S6 and added to S5. The same series dilution was performed for S4-S1. All dilutions were refrigerated until use (FIG. 4).

ELISA detecting IFN-β was performed using VeriKine Human IFN-β ELISA Kit (Cat 41410, pbl assay science). 1 ml of the culture supernatant was transferred to a new tube and centrifuged for 5 minutes at 3000 rpm. The culture supernatant was then transferred to a new tube and stored at −80° C. until use.

A standard curve was generated: Seven polypropylene tubes were labeled (S1-S7). Using polypropylene tips, 250 μl Standard Diluent was added to each tube, except S7 where 492.5 μl Standard Diluent was added. 10 μl of the Human IFN-β Standard was added to 90 μl of Standard Diluent. 7.5 μl of prediluted standard was added to S7 and mixed thoroughly. 250 μl of S7 was transferred to S6 and mixed thoroughly. The same series dilution was performed for the reset S5-S1. All dilutions were set aside until use.

The number of microplate strips required to test the desired number of samples were determined plus the appropriate number of wells needed to run blanks and standards. 100 μl of the interferon standard, blank or sample were added to each well, covered with a plate sealer and incubated for 1 hour. After 1 hour, the contents of the plate were emptied and the wells were washed for one time only with diluted Wash Buffer. 100 μl of diluted Antibody Solution was added to each well, covered with a plate sealer and incubated for 1 hour. After 1 hour, the contents of the plate were emptied and the wells were washed for three times with diluted Wash Buffer. 100 μl of diluted HRP Solution was added to each well, covered with a plate sealer and incubated for 1 hour. During this incubation period, the TMB Substrate Solution was warmed to RT (22-25° C.). After 1 hour, the contents of the plate were emptied and the wells were washed for four times with diluted Wash Buffer. 100 μl of the TMB Substrate Solution was added to each well and incubate, in the dark, at RT (22-25° C.), for 15 minutes. A plate sealer was not used during the incubation. After the 15 minutes incubation of TMB, 100 μl of Stop Solution was added to each well. Using a microplate reader, the absorbance at 450 nm was determined within 5 minutes after the addition of the Stop Solution.

In some embodiments, 293T cells are transiently transfected with a tested RNA in a formulation/carrier as disclosed herein, for example using the transfection method as described above. IFN-α and IFN-β release of the transfected cells are measured, for example, using a method as described above.

### Results

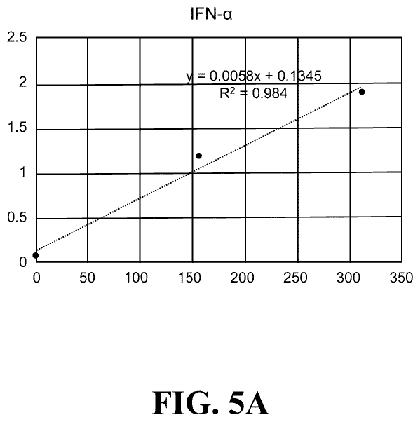

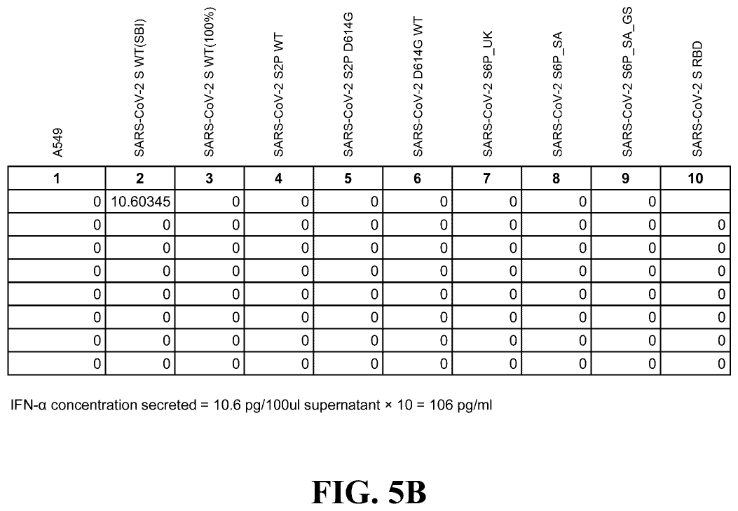

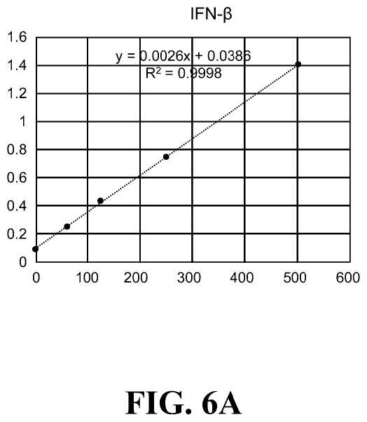

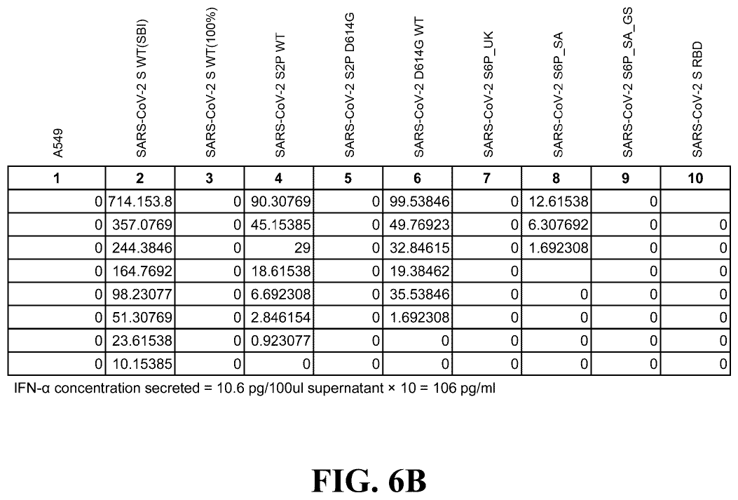

To assess IFN-α and IFN-β secretion from A549 cells, ELISA was performed as described herein. A representative resultant ELISA plate was photographed and is shown in FIG. 4, while the results were further quantified as show in FIGS. 5-6. The data demonstrated that interferon-α (IFN-α or IFN-Alpha) only secreted from the A549 cells transfected a unmodified SARS-COV-2 Spike mRNA(SBI) and interferon-β (IFN-β or IFN-Beta) secreted from the A549 cells transfected an unmodified SARS-COV-2 Spike mRNA(SBI), S2P WT, D614G WT and S6P_SA.

### Conclusions

Interferon-α only secreted from the A549 cells transfected an unmodified SARS-COV-2 Spike mRNA(SBI). Interferon-β secreted from the A549 cells transfected an unmodified RNA encoding the SARS-COV-2 Spike WT(SBI), or SARS-COV-2 Spike S2P WT, or SARS-COV-2 Spike D614G WT, or SARS-COV-2 Spike S6P_SA. Accordingly, without wishing to be bound by the theory, the observed lack of spike protein expression in A549 cell transfected with an unmodified RNA encoding the SARS-COV-2 Spike WT(SBI) (data not shown) may be caused by the activation of strong cGAS-STING pathway (innate immunity) which inhibits spike protein translation.

### Example 10: Evaluation of Immunogenicity and Efficacy of a SARS-COV-2 Spike mRNA Vaccine Against SARS-COV-2 in K18-hACE2 Mice

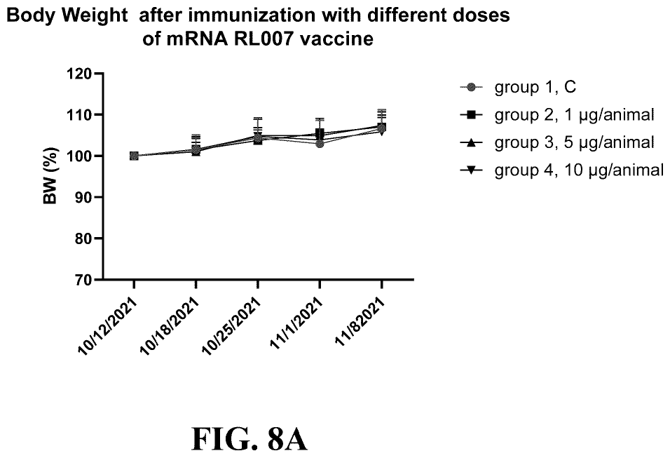

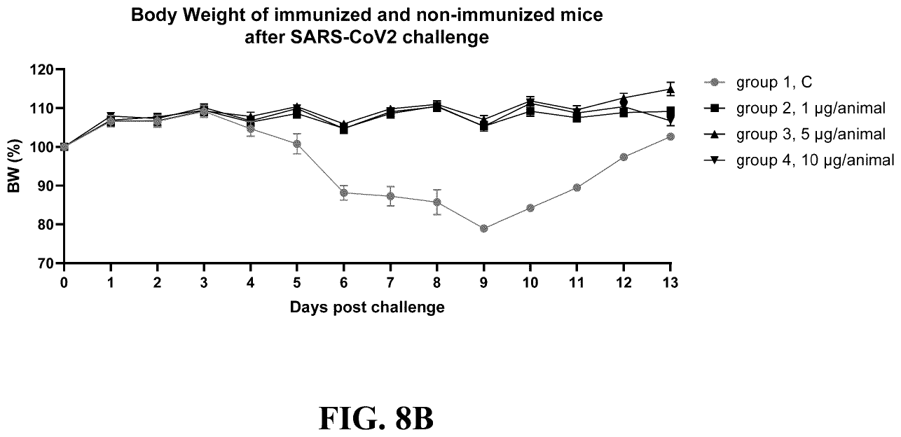

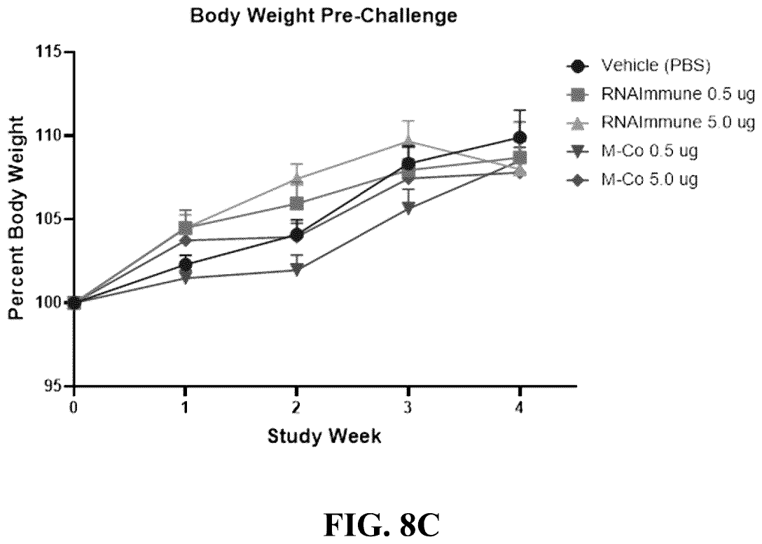

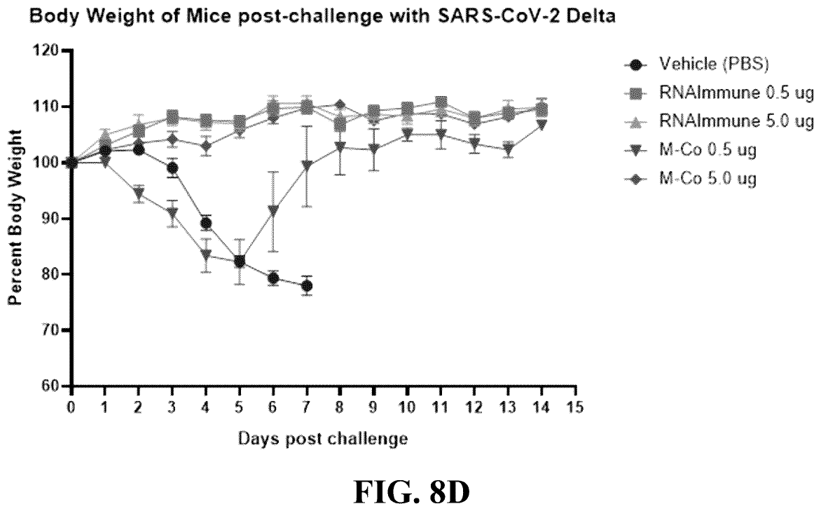

A total of forty (40) six- to eight-week-old (6-8) female K18-hACE2 mice were purchased from Jackson Laboratory. All animal experiments were conducted at the Noble Life Science in collaboration with George Mason University (GMU). The study was conducted without direct involvement of Quality Assurance but adhered to applicable NLS SOPs and following the guidelines and approval of the Institutional of Animal Care and Use Committee (IACUC), GMU Biomedical Research Laboratories. Mice were assigned to four (4) groups consisting of ten (10) mice each following an acclimation period. Table 3 shows dosing groups for SARS-COV-2/human/ITA/INMI1/2020 challenge. Table 4 shows dose groups for ARS-COV-2 B.1.1617.2 delta variant. On Day 0 and Day 14, animals in group one (1) and were inoculated intramuscularly with vehicle and mice in groups two (2), three (3), and four (4) were inoculated intramuscularly with escalating doses of a representative RL-007 mRNA vaccine formulation, prepared according to Example 3, comprising SEQ ID NO: 52. This vaccine formulation is designated “RV-1730” and uses an RL-007 carrier. Blood was collected from the mice on Days −1, 13, and 27 and serum samples were used for plaque-reduction neutralization test (PRNT). On Day 28, animals were challenged intranasally with 5.5×10⁴ PFU/mouse SARS-COV-2/human/ITA/INMI1/2020 wild type variant (NLS-GMU-WB2-042121) (FIG. 8A and FIG. 8B) or 1.5×10⁴ PFU/mouse SARS-CoV-2 B.1.1617.2 Delta variant (FIG. 8C and FIG. 8D). All mice were monitored daily for mortality, body weight, body temperature and scored for clinical signs of distress for thirteen (13) days after challenge. Mice that had a clinical severity score of >10 or lost >20% of their pre-challenge body weight were humanely euthanized and recorded as non-survivors. All surviving mice were euthanized thirteen (13) days after challenge. A portion of lung tissue was harvested from euthanized mice, placed into 10% formalin and shipped to Histoserv for histopathological assays by H&E staining.

TABLE 3
Dosing Groups for SARS-CoV-2/human/ITA/INMI1/2020 challenge study

| | | | | | | | | | | Post-SARS- |
|---|---|---|---|---|---|---|---|---|---|---|
| | | Animal | | Injection | | | Transfer | Serum | Challenge | CoV-2 |
| | Mouse | Number/ | | Volume | | | mice to | Samples | Virus | challenge |
| Group | Strain | Sex | Test Article | (mL) | Route | Frequency | GMU | for PRNT | (D 28) | monitoring |
| 1 | K18- | 10/ | 1xPBS | 50 | I.M. | D 0, D 14 | D 21 | D −1, D 13, | SARS- | D 28 to D41 |
| 2 | hACE2 | Female | RL007 mRNA | | | | | D 27 | CoV-2/ | |
| | Mice | | vaccine 20 | | | | | | human/ | |
| | | | mg/mL (1 | | | | | | ITA/ | |
| | | | mg/mouse) | | | | | | INMI1/ | |
| 3 | | | RL007 mRNA | | | | | | 2020 | |
| | | | vaccine 100 | | | | | | | |
| | | | mg/mL (5 | | | | | | | |
| | | | mg/mouse | | | | | | | |
| 4 | | | RL007 mRNA | | | | | | | |
| | | | vaccine 200 | | | | | | | |
| | | | mg/mL (10 | | | | | | | |
| | | | mg/mouse) | | | | | | | |

TABLE 4
Dose Groups for ARS-CoV-2 B.1.1617.2 Delta variant

| | | | | | | | | Challenge | Post- SARS- |
|---|---|---|---|---|---|---|---|---|---|
| | | Animal | | | | Transfer | Serum | Virus | CoV-2 |
| | Mouse | Number/ | Test | | | mice to | Samples | (D 27, | challenge |
| Group | Strain | Sex | Article | Route | Frequency | GMU | for PRNT | D 28) | monitoring |
| 1 | K18- | 15/ | 1xPBS | I.M. | D 0, D 14 | D 21 | D −1, D 13, | SARS- | D 28 to D 42 |
| 2 | hACE2 | Female | RNAImmune | | | | | CoV-2 | |
| | Mice | | mRNA | | | | D 27 | B.1.1617.2 | |
| | | | vaccine Low | | | | | Delta | |
| | | | Dose (0.50 | | | | | variant | |
| | | | μg/mouse) | | | | | | |
| 3 | | | RNAImmune | | | | | | |
| | | | mRNA | | | | | | |
| | | | vaccine High | | | | | | |
| | | | Dose (5 | | | | | | |
| | | | μg/mouse) | | | | | | |
| 4 | | | m-Co control | | | | | | |
| | | | vaccine Low | | | | | | |
| | | | Dose (0.50 | | | | | | |
| | | | μg/mouse) | | | | | | |
| 5 | | | M-Co | | | | | | |
| | | | control | | | | | | |
| | | | vaccine High | | | | | | |
| | | | Dose (5 | | | | | | |
| | | | μg/mouse) | | | | | | |

Mice were monitored daily for up to fourteen (14) days post challenge for mortality, clinical signs of distress, body weight, and body temperature. A severity score for clinical distress was recorded daily for each mouse based on a GMU-IACUC approved Animal Study Clinical Monitoring Chart (Table 5). Briefly, a score from 0 (normal) to 3 (severely sick) in four (4) observable phenotypic categories 1) Appearance, 2) Mobility, 3) Attitude, 4) Respiratory Distress, and body weight were recorded daily for each individual mouse. Mice that had a cumulative score of 10 or greater were humanely euthanized.

TABLE 5
Clinical Severity Scoring Matrix

| | | | Respiratory | Body | Total |
|---|---|---|---|---|---|
| Appearance | Mobility | Attitude | Distress | Weight | Score |
| 0 - Smooth | 0 - Active/ | 0 - Alert | 0 - Normal | 0 = Normal | 0-5 and all |
| coat, bright | Scurrying | | | | categories ≤1 = |
| eyes | | | | | Normal |
| | | | | | 1X daily |
| | | | | | monitoring |
| 1 - Slightly | 1 - Walking | 1 - Alert, | 1 - Ocular | 1 = 0 to | 6-9 or any |
| scruffy | | playful when | and nasal | 10% weight | single |
| and/or | | stimulated | discharge, | loss | score >1 = |
| hunched | | | Sneezing | | 2X daily |
| at rest | | | | | monitoring |
| 2 - Scruffy | 2 - Slow | 2 - Alert, not | 2 - Heavy | 2 = 10-20% | ≥10 or any |
| and/or | movement | playful when | ocular and | weight | single score = |
| hunched | | stimulated | nasal | loss | 3 = |
| at rest, | | | discharge, | | Euthanize |
| watery | | | cough and | | |
| eyes | | | wheezing | | |
| 3 - Very | 3 - No movement/ | 3 - Not alert, | 3. Severe | 3 = Greater | FD = Found |
| scruffy | Unresponsive | not playful | respiratory | than 20% | Dead |
| and/or | when touched/ | | distress, | weight | |
| hunched, | stimulated | | Open | loss | |
| | | | mouth | | |
| | | | breathing | | |

The neutralizing titers of serum specimens collected on Study Day −1, Day 13, and Day 27 were assessed using plaque reduction neutralization titration (PRNT). All sera were heat inactivated by incubation at 56° C. for 30 minutes prior to testing. Six serial two-fold dilutions of each serum were prepared in EMEM. Dilutions ranged from 1:4 to 1:3200 and were adjusted to account for increased neutralizing activity at different time points and treatment groups. Twenty-five microliters (25 μL) of each dilution was combined with 25 μL of SARS-COV-2 B.1.1617.2 Delta variant BEI: NR-55672 viral stock, mixed and incubated at 37° C. for one (1) hour. Supernatant was removed from Vero-E6 cells seeded the night before in 12 well plates and replaced with 300 μL of Eagle Minimum Essential Medium (EMEM) in each well. The incubated virus+diluted serum mixtures were added on top of Vero E6 monolayer and plates were incubated at 37° C./5% CO₂ for 1 hour. Plates were shaken every 10-15 minutes during incubation. After one-hour incubation, 1.5 ml overlayer containing 2×EMEM and 0.6% agarose at a ratio of 1:1 was added to the plates. Plates were incubated for 72 hours at 37° C./5% CO₂ after solidification of agarose at room temperature. Following incubation, cells were fixed by adding 0.5 ml of 10% formaldehyde into each well on top of the agarose and incubating at room temperature overnight. The palettes of agarose were gently removed, and the cellular monolayers were stained for 10 to 15 minutes with the addition of 0.5 ml of 1% crystal violet. After staining, the plates were washed with water to remove excess stain solution and plaques were manually enumerated for each viral dilution. The neutralizing titers were calculated as the reciprocal of the lowest dilution that resulted in a greater than 50% reduction (PRNT₅₀) or 90% reduction (PRNT₉₀) in p.f.u. (plaque forming units) relative to negative control sera. Human sera collected from two fully vaccinated males and serum from naïve mice were used as a positive and negative controls, respectively.

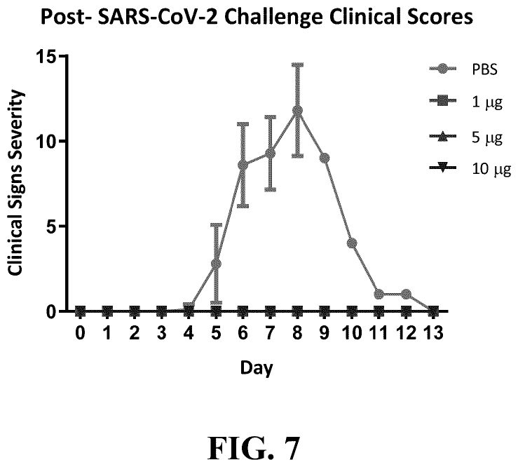

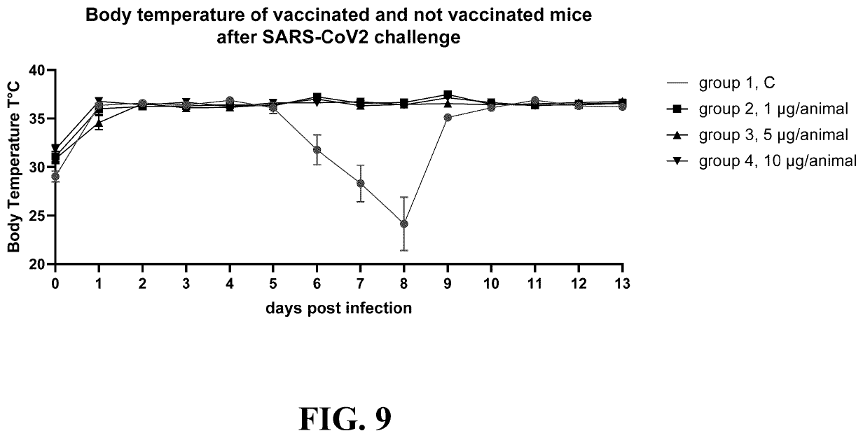

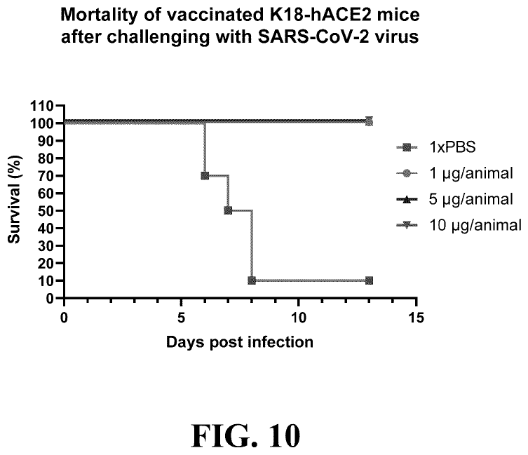

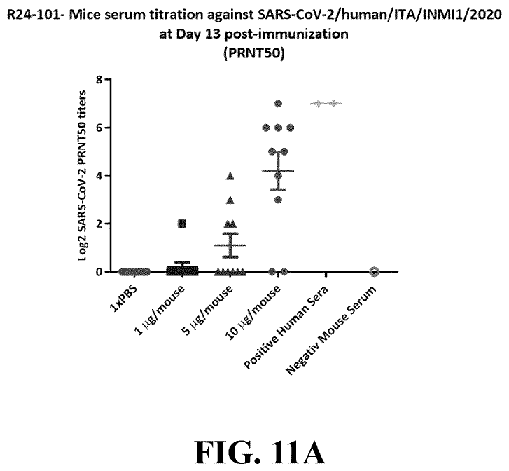

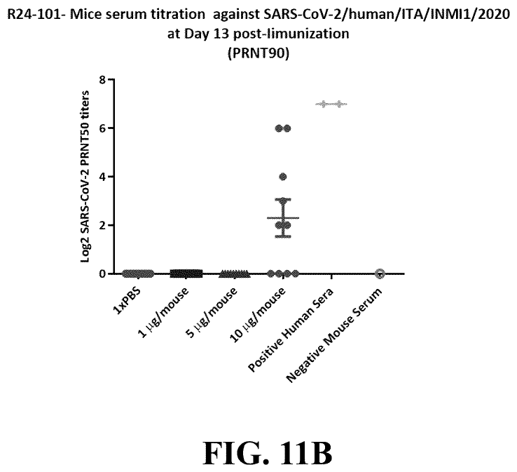

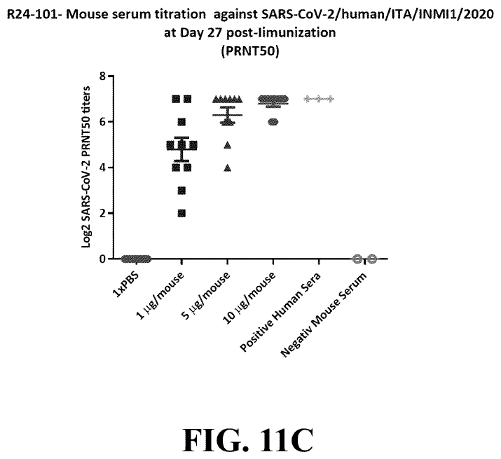

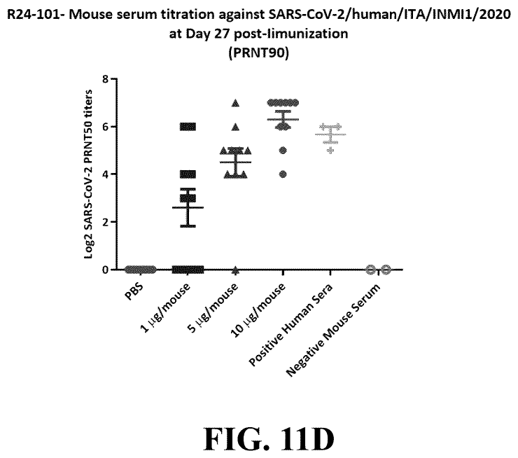

Results: All mice immunized with RL-007 (Group 2, 3, and 4 mice) exhibited no increase in clinical sign severity following challenge with SARS-COV-2. In contrast, Group 1 mice exhibited sharp increases in clinical sign severity following SARS-COV-2 challenge (FIG. 7). The SARS-COV-2 spike mRNA vaccine was shown to have no effect on group 2, 3, or 4 mouse bodyweight (FIG. 8A). Following SARS-COV-2 challenge, mice administered all mice immunized with SARS-COV-2 spike mRNA vaccine (Group 2, 3, and 4 mice) exhibited no change in bodyweight as compared to the control (group 1) mice, which exhibited declining bodyweight at least four days after SARS-COV-2 challenge (FIG. 8B). A similar trend was observed following viral challenge with SARS-COV-2 B.1.1617.2 Delta variant (FIG. 8C and FIG. 8D). Mouse body temperatures followed similar trends, where SARS-COV-2 spike mRNA vaccine-immunized mice showed stable body temperatures over the days following SARS-COV-2 challenge, whereas the control mice experienced decreased body temperatures at least five days following virus challenge (FIG. 9). All SARS-COV-2 spike mRNA vaccinated mice survived at least up to 13 days following SARS-COV-2 challenge (FIG. 10). 10% of control mice survived at least 8 days post challenge (FIG. 10). High neutralizing antibody titers were observed at least 13 (FIG. 11A and FIG. 11B) and 27 (FIG. 11C and FIG. 11D) days post-immunization.

Conclusions: These data suggest that the SARS-COV-2 spike mRNA vaccines provided herein are capable of preventing a subject from having symptoms (e.g., clinical sign severity, changes in body temperature, and/or changes in body weight) of or symptomatic SARS-COV-2 infection. As demonstrated herein, immunization with the SARS-COV-2 spike mRNA vaccine is capable inducing an immune response to SARS-COV-2 in a subject by, e.g., increasing neutralizing antibody titers against SARS-COV-2 following immunization for a period of at least 13 or at least 27 days. The provided SARS-COV-2 spike mRNA vaccines can therefore treat subjects infected with SARS-COV2, and reduce a subject's SARS-COV-2 viral load.

### Example 11: Viral Titers in Nasal Passages and Lung Tissues

A portion of right lung tissue of each mouse euthanized was collected and placed into 10% formalin. Lung tissue samples fixed with formalin were removed from ABSL3 and shipped to Histoserv (19526 Amaranth Dr, Germantown, MD 20874) for histopathological assays. A portion of lung tissue for each of the mice designated for euthanasia on Day 3 post challenge was placed into cell culture medium for analysis of viral titers in lung tissues. Viral titers in nasal passages and lung tissues were determined in five mice (5) from each group three (3) days post viral challenge. Mice were anesthetized by inhalation of isoflurane and nasal washes were collected from each mouse in a total volume of 100 mL of sterile nasal wash buffer for determination of viral replication and shedding in the upper respiratory tract (pfu/mL). Mice were humanely euthanized under a heavy plane of anesthesia and lung tissues were collected. Tissue from the right lung lobe was placed into tissue culture medium, homogenized and used for determination of viral titer in the lower respiratory tract. Serial ten-fold dilutions of each nasal wash or lung homogenate were prepared in EMEM. Supernatant was removed from Vero-E6 cells seeded the night before in 12 well plates and replaced with 300 μL of diluted nasal wash or lung homogenate in each well. The plates were incubated at 37° C./5% CO₂ for 1 hour. Plates were shaken every 10-15 minutes during incubation. After one-hour incubation, 1.5 mL overlayer containing 2×EMEM and 0.6% agarose at a ratio of 1:1 was added to the plates. Plates were incubated for 72 hours at 37° C./5% CO₂ after solidification of agarose at room temperature. Following incubation, cells were fixed by adding 0.5 mL of 10% formaldehyde into each well on top of the agarose and incubating at room temperature overnight. The palettes of agarose were gently removed, and the cellular monolayers were stained for 10 to 15 minutes with the addition of 0.5 ml of 1% crystal violet. After staining, the plates were washed with water to remove excess stain solution and plaques were manually enumerated for each viral dilution. Viral titers were calculated as pfu/mL.

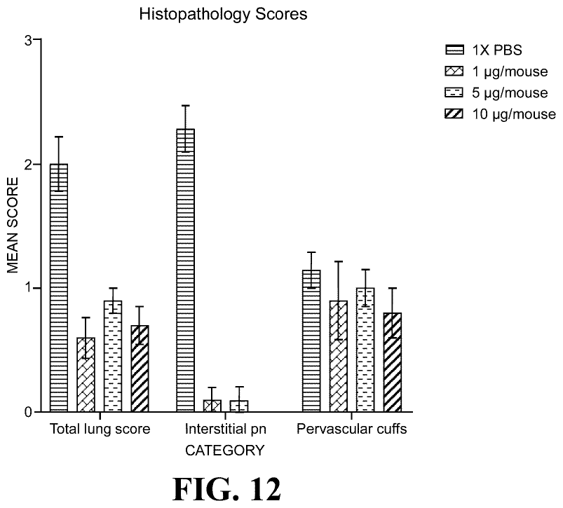

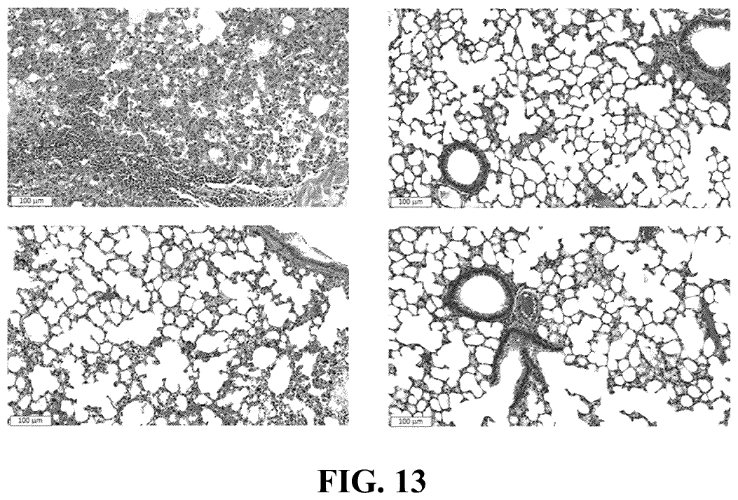

Results and Conclusions: Histopathology images for representative lung tissues harvested 13 days post-challenge with SARS-COV-2 are shown in FIG. 13. Histopathology scores were determined based upon total lung score, interstitial pneumonia, and perivascular cuffs (FIG. 12). Mice immunized with SARS-COV-2 spike mRNA vaccine had low mean histopathology scores for each of the three measures when compared to wild type mice, indicating that SARS-COV-2 spike mRNA vaccine provides protection against tissue level symptoms and damage onset by SARS-COV-2 infection.

### Example 12: Immunogenicity of Serum Immunized with RL007

To assess immunogenicity of SARS-COV-2 delta variant, RV-1730, in mice, naïve 6- to 8-week-old female BALB/c mice were randomly divided into groups of five-eight mice and vaccinated via intradermal injection with RL007 (1, 5, 10 and 20 μg per mouse). All mice were immunized twice at 21-day intervals. Blood was taken before each immunization at 14 and 35 days after the first immunization via tail bleed or cardiac puncture. The blood was allowed to clot overnight at 4° C. before the serum was harvested by centrifugation at 10,000×g for 10 min at 4° C. Samples were stored at 4° C. until further analysis. To determine S1- and RBD-specific serum IgG titers, MaxiSorp plates (BioLegend) were coated with 100 μl of recombinant S1 or RBD (1 μg/ml) in sodium carbonate buffer overnight at 4° C. The wells were washed 3 times with PBS-T and incubated the plates with 200 μl of blocking buffer for 2 hours at RT. After washing 3 times with PBS-T, the plate was incubated with 100 μl of sera for 2 hours at RT. And the plate washed 5 times with PBS-T and incubated with HRP-conjugated secondary antibody for 1 hour at RT. Finally, the plate was washed 5 times with PBS-T and incubated the plates with 100 μl of 1×TMB substrate for 10 minutes. The reaction was stopped by adding 100 μl of 1N—HCl and read at 450 nm using the Cytation7. For reciprocal endpoint IgG titers, 2-fold serial diluted sera were added to the wells immobilized with different spike proteins. All procedures were the same as described above.

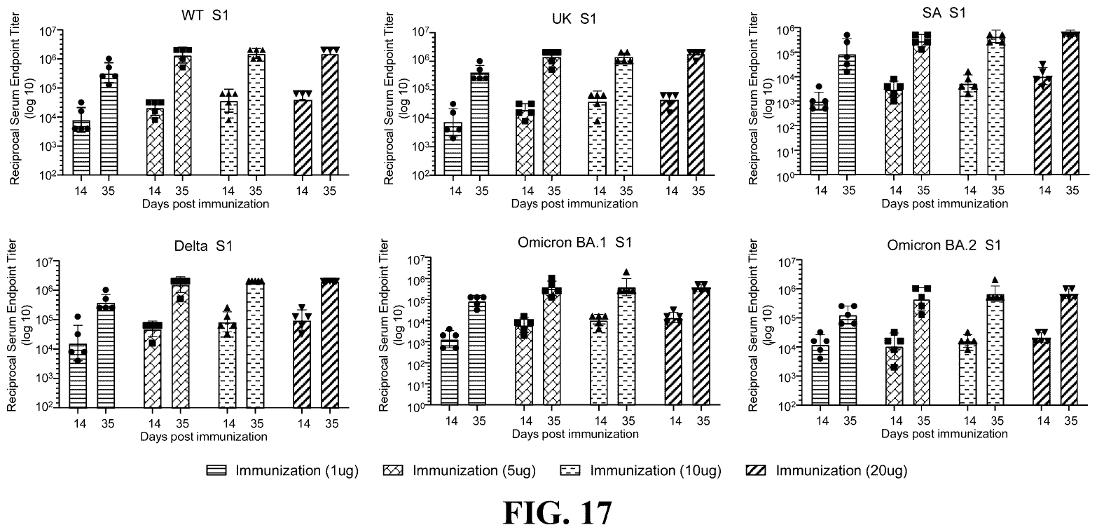

Results and Conclusions: Increased endpoint IgG titers of RL007 immunized sera against different variant spike proteins was observed after first and second immunization doses (FIG. 17). The SARS-COV-2 spike mRNA vaccine is therefore capable inducing an immune response against different SARS-COV-2 variant spike proteins in a subject by, e.g., increasing IgG titers against SARS-COV-2 following first and second immunization doses for a period of at least 14 or at least 35 days.

### Example 13: Determination of Neutralizing Antibody Titers of Serum Immunized with RL007

To determine the titers of neutralizing antibodies, we first generated SARS-CoV-2-Psudovirus Particles. In brief, murine Leukemia Virus (MLV) particles pseudotyped with a SARS-COV-2 Spike protein construct were generated in HEK293T (Cat #CRL-3216, ATCC) cells, following a protocol described previously for SARS-COV1,2 with some modification. All the plasmid DNAs were purified with ZymoPURE II Plasmid Midiprep Kit (Cat #D4201, Zymo Rescarch). In brief, 8 million HEK293T cells were plate into a 10-cm tissue culture dish (Cat #sc-251460, Santa Cruz) in 16 ml DMEM (Cat #25-500, Genesee Scientific)+10% FBS (Cat #35-010-CV, Corning Life Sciences) without any antibiotics. On the 2nd day, the cells were transfected with 8 μg pTG-Luc, 6 μg pCMV-MLVgag-pol and 6 μg pcDNA3.1-SARS-COV-2-Spike-□C19 of different variants using Lipofectamine 3000 reagent (Cat #L3000015, ThermoFisher). The cells were cultured for an additional 48 hr. The supernatant was collected into a 50-ml Falcon tube and spun at 290×g for 7 min. The supernatant (pseudotyped virus solution) was then passed through a 0.45 μm filter (Cat #sc-358814, Santa Cruz) using appropriate syringe. The pseudotyped virus solution was then aliquoted into cryovials and stored at −80° C. Each 10-cm cell culture dish produces about 16 ml SARS-COV-2-PP. The SARS-COV-2-PP was tested for the quality control with HEK293-ACE2 cell line (created at Codex BioSolutions). For neutralizing assay with pseudovirus, the serum was heat inactivated by incubate the serum at 56° C. for 30 min. The day before the infection, 7.5×10³ HEK293-ACE2 cells were plated into a 384-well white clear plate (Cat #353963, Corning Life Sciences) precoated with Poly D Lysine (Cat #3439-100-1, Trevigen, Inc) in 15 μl culture medium (DMEM+10% FetalClone II Serum, Cat #SH3006603, Fisher Scientific). The cell plate was placed in a CO₂ incubator)(37° C. On the 2nd day, the serum to be tested was diluted in the culture medium on a 96-well compound plate. 65 μl of SARS-CoV-2 MLV pseudovirus particles (pp) were mixed with 26 μl of the testing sample prepared above and incubated at 37° C. for 1 hr. After the medium in each well of 384-well cell plate was removed, 17.5 μl of each serum-pp mixture was added into each well. The plate was centrifuged at 54×g for 15 min at 4° C. and additional 7.5 ml of the culture medium was then added into each well. Luciferase activities were measured with Firefly Luciferase Assay Kit (CB-80552-010, Codex BioSolutions Inc). IC₅₀ values were calculated based on curve fitting in GraphPad Prism (data was normalized as the percentage of infectivity).

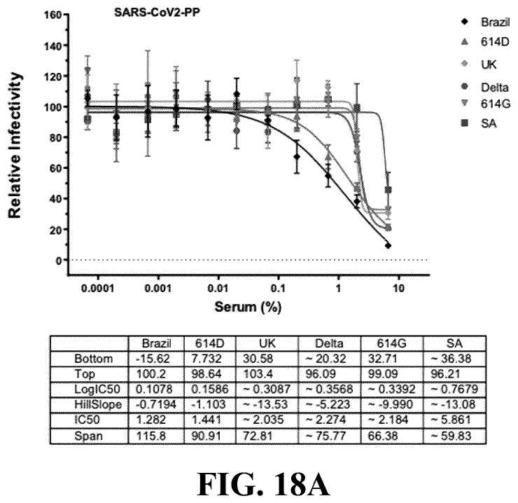

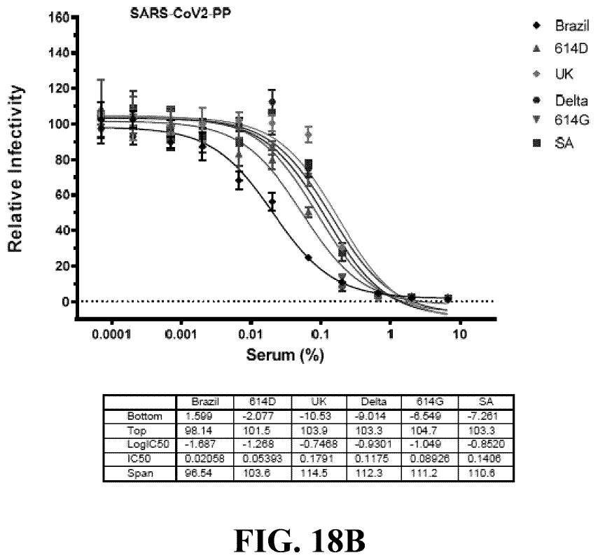

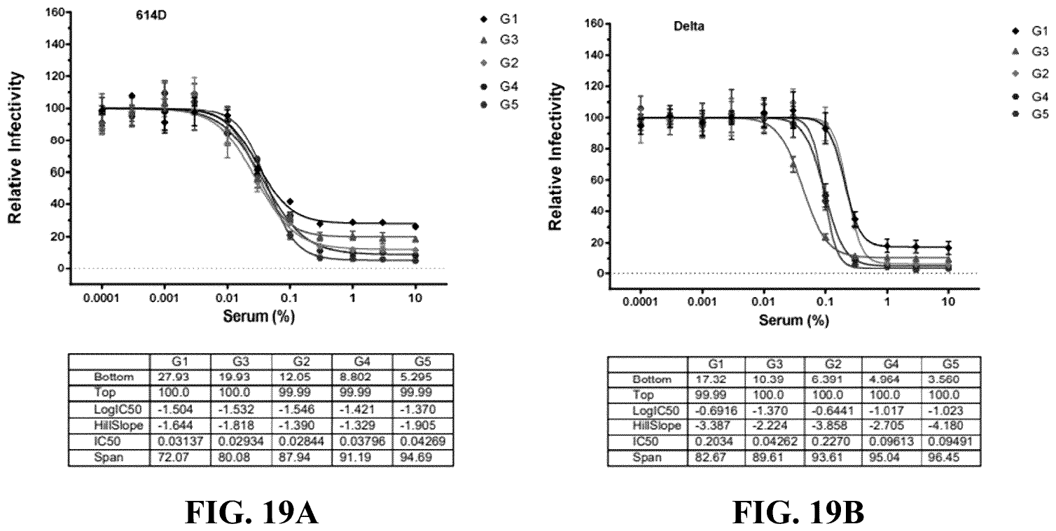

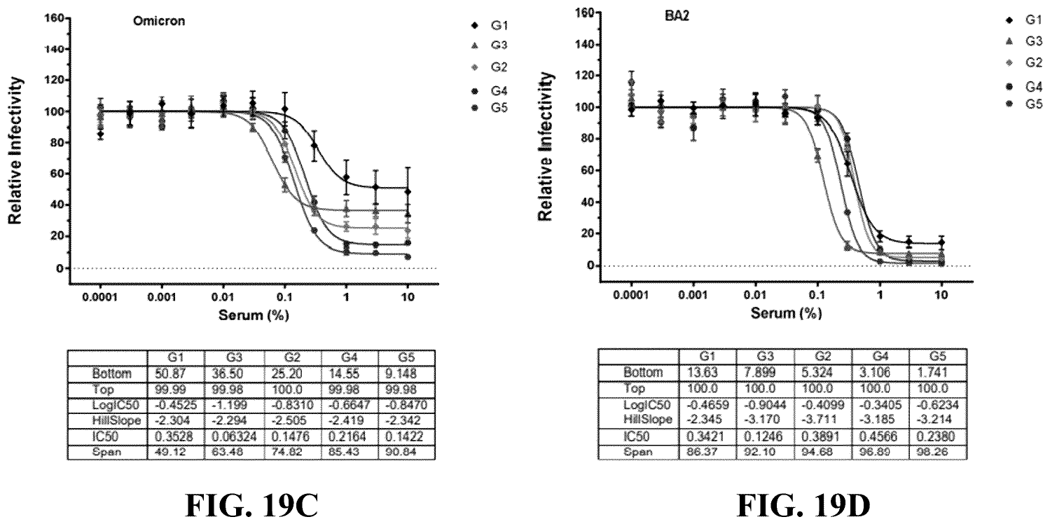

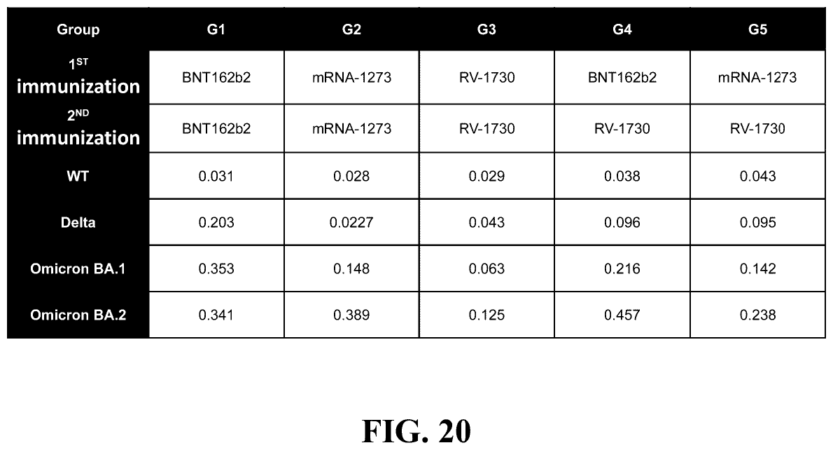

Results and Conclusions: Neutralization antibody titers against SARS-COV-2 pseudovirus particles were measured following first and second immunization doses. IC₅₀ values were calculated based on curve fitting. Strain-specific neutralization was observed. Curves and IC₅₀ values are shown in FIG. 18A, FIG. 18B, FIG. 19A, and FIG. 19B. IC₅₀ values are tabulated in FIG. 20 for each of the neutralization assay conditions provided. These data demonstrate that the SARS-COV-2 spike mRNA vaccine provided herein is capable of inducing an immune response against different SARS-COV-2 variant spike proteins in a subject infected with SARS-COV-2 by, e.g., increasing neutralizing antibody titers against SARS-COV-2 following at least 14 or 35 days after first and second immunization doses.

### Example 14: Intracellular Cytokine Staining

Spleens from mice were collected and processed into single-cell suspensions in RPMI1640 media supplemented with 10% heat-inactivated fetal calf serum and penicillin/streptomycin (R10 media). Red blood cells were lysed using RBC lysis buffer (KD Medical) and resuspended in R10 media to stop the lysis. Splenocytes were counted and 200,000 cells per well were added to 96 well plates. Cells were then stimulated for 72 hours at 37° C. with 2 μg of PepMix™ SARS-COV-2 (Spike B.1.617.2/Delta) (JPT Peptide Technologies GmbH) or media alone as a negative control. The plates were centrifuged, and supernatant was collected and frozen at −80° C. for cytokine detection. Measurements and analyses of secreted cytokines from a murine 11-plex kit were performed using a multiplex bead-based technology (Luminex) assay with a Luminex 100/200 instrument (Luminex) after 2-fold dilution of supernatants.

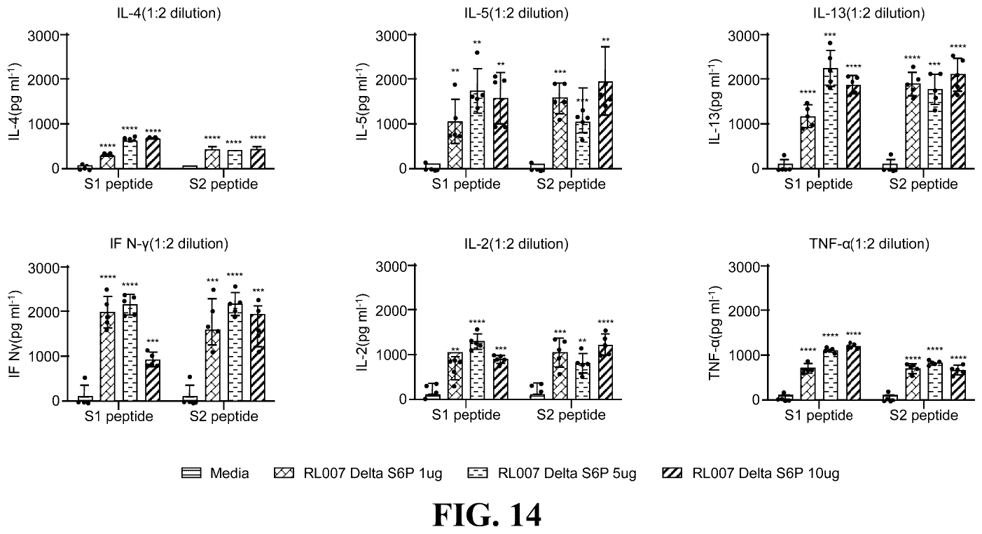

Results and Conclusions: After 72 hours, the culture supernatants were harvested by centrifugation and the secreted Th1-cytokines (IFN-γ, IL-2, TNF-α) and Th2-cytokines (IL-4, IL-5, IL-13) were measured. Stimulation with SARS-COV-2 S proteins increased the secretion of Th1-cytokines (IFN-γ, IL-2, TNF-α) and Th2-cytokines (IL-4, IL-5, IL-13 in all SARS-COV-2 spike mRNA vaccine-treated mice (FIG. 14). These data indicate that SARS-COV-2 spike mRNA vaccines of the present disclosure induce a balanced Th1/Th2 immune response against SARS-COV-2 in a subject infected with SARS-COV-2 by, e.g., inducing cytokine release against SARS-COV-2 infection.

### Example 15: IFN-γ ELISPOT Analysis on Splenocytes

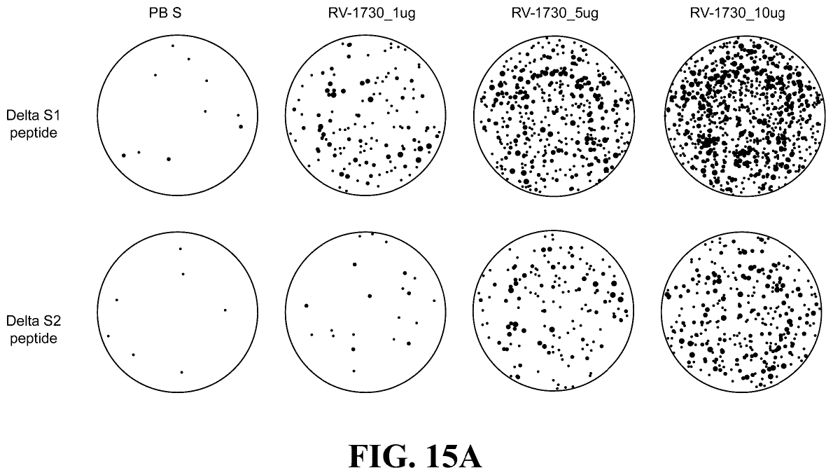

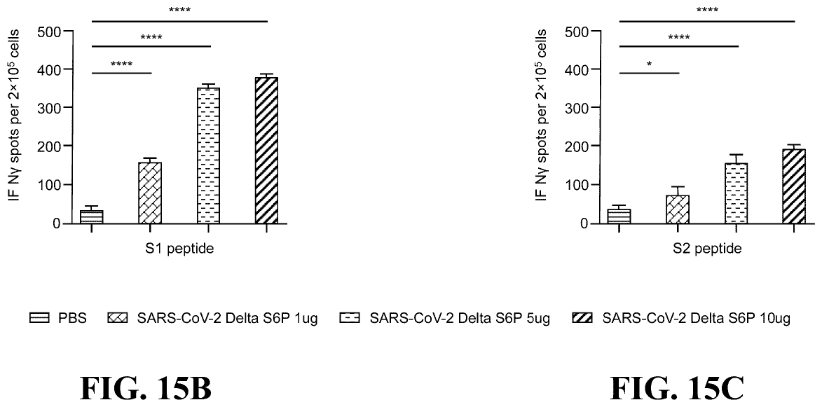

Spleens from mice were collected and processed into single-cell suspensions in RPMI1640 media supplemented with 10% heat-inactivated fetal calf serum and penicillin/streptomycin (R10 media). Red blood cells were lysed using RBC lysis buffer (KD Medical) and resuspended in R10 media to stop the lysis. Splenocytes were counted and 200,000 cells per well in a 6-well plate and were added to the plates of the Mouse IFN-g ELISpotPLUS (HRP) kit (Mabtech). Cells were then stimulated for 16 hours at 37° C. with 2 μg of PepMix™ SARS-COV-2 (Spike B.1.617.2/Delta) (JPT Peptide Technologies GmbH) or media alone as a negative control. Spots were developed as per the manufacturer's instructions. Spots were quantified by Cytation7 and are shown in FIG. 15A.

Results and Conclusions: Increased amounts of IFN-γ spots were present in cells from SARS-COV-2 spike mRNA vaccinated mice compared to unvaccinated control mice (FIG. 15B and FIG. 15C). These data indicate that the SARS-COV-2 spike mRNA vaccines of the present disclosure can induce an immune response against SARS-COV-2 in a subject, by, e.g., increasing IFN-γ levels in the subject.

### Example 16: Th1/Th2 Cytokine Analysis of Memory T Cells

Following re-stimulation with peptide pools (S1 and S2) corresponding to the S protein, the culture supernatants of splenocytes from RV-1730-immunized mice was assessed the secreted cytokines using Th1/Th2 Cytokine 11-plex mouse procartaplex panel. In brief, nine weeks post-boost, splenocytes were isolated from 5 mice per group and re-stimulated with no peptides or pools of overlapping peptides from SARS-COV-2 S protein in the presence of a protein transport inhibitor cocktail. After 16 hours, intracellular cytokine staining (ICS) was performed to quantify CD4⁺ and CD8⁺ T cell responses. Cytokine expression in the presence of no peptides was considered background and subtracted from the responses measured from the S1 and S2 peptide pools for each individual mouse.

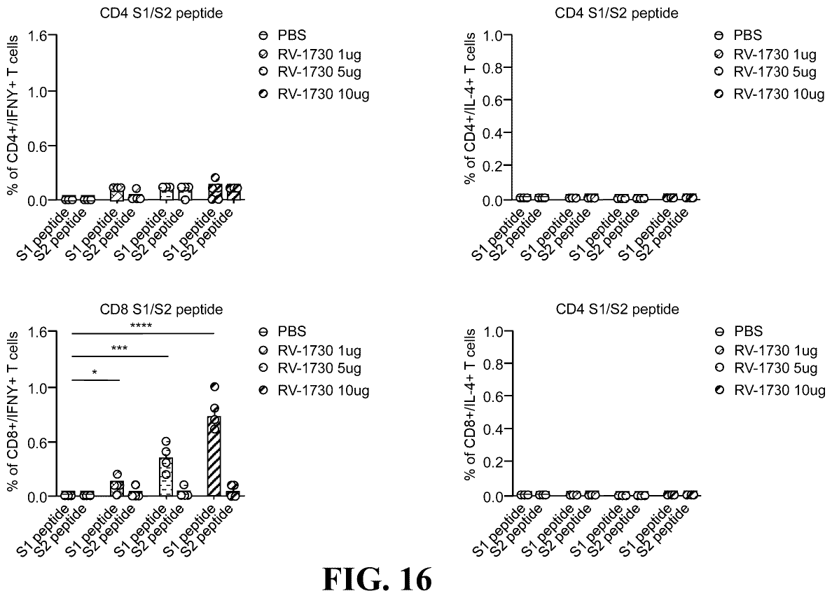

Results and Conclusions: CD4⁺/IFN-γ and CD8⁺/IFN-γ T cell responses were increased in culture supernatants of splenocytes from SARS-COV-2 spike mRNA vaccinated mice compared to unvaccinated control mice (FIG. 16). The SARS-COV-2 spike mRNA vaccines of the present disclosure can induce an immune response against SARS-CoV-2 in a subject, by, e.g., increasing CD4⁺/IFN-γ and CD8⁺/IFN-γ T cell responses in the subject. RV-1730 induces robust neutralizing activity and CD8 T cell responses, balanced Th1/Th2 antibody isotype responses, as well as cytokine release from T cells by ex vivo stimulation with SARS-COV-2 spike protein peptides. This broad spectrum effectiveness in inducing a strong cellular immune response provides the unique capability to not only use RV-1730 against current/existing SARS-COV-2 variants and descendent lineages therein, but also to implement RV-1730 against any new SARS-COV-2 variants of concern that may arise in the future.

TABLE 8
SEQUENCES

| | Amino Acid | DNA | RNA |
|---|---|---|---|
| Designation | Sequence | Sequence | Sequence |
| S protein | SEQ ID NO: 1 | — | — |
| S protein D614G | SEQ ID NO: 2 | SEQ ID NO: 3 | SEQ ID NO: 4 |
| and S6P | | (non-optimized) | (non-optimized) |
| deltaC S2P | SEQ ID NO: 5 | | |
| deltaC S2P WC | SEQ ID NO: 6 | | |
| deltaC S6P | SEQ ID NO: 7 | SEQ ID NO: 8 | SEQ ID NO: 9 |
| deltaC S6P WC | SEQ ID NO: 10 | | |
| deltaC S6P plus | SEQ ID NO: 11 | SEQ ID NO: 12 | SEQ ID NO: 13 |
| DPCh | SEQ ID NO: 14 | SEQ ID NO: 15 | SEQ ID NO: 16 |
| 3′UTR | — | SEQ ID NO: 17 | SEQ ID NO: 18 |
| β-globulin 5′UTR | — | SEQ ID NO: 19 | SEQ ID NO: 20 |
| SYS UTR 2.0 | — | SEQ ID NO: 21 | SEQ ID NO: 22 |
| SYS UTR 1.0 | — | SEQ ID NO: 23 | SEQ ID NO: 24 |
| SYS4 5′UTR | — | SEQ ID NO: 25 | SEQ ID NO: 26 |
| polyA 40 | — | SEQ ID NO: 56 | SEQ ID NO: 27 |
| polyA 60 | — | SEQ ID NO: 57 | SEQ ID NO: 28 |
| polyA signal HSV | — | SEQ ID NO: 29 | SEQ ID NO: 30 |
| 5′UTR-deltaC S6P- | | SEQ ID NO: 31 | SEQ ID NO: 32 |
| 3′ UTR-polyA | | | |
| Plasmid | — | SEQ ID NO: 33 | — |
| Side chains of HKP | SEQ ID NO: 34 to 47 | — | — |

SEQ ID NO: 1, amino acid sequence of S protein:
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKC
VN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNN
SYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQAL
NTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVY
DP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDED
DSEPVLKGVKLHYT
SEQ ID NO: 2, amino acid sequence of S protein comprising
D614G (marked in bold font with underlines) and S6P
mutations (F817P, A892P, A899P, A942P, K986P, and V987P,
marked in bold and italic font with underlines):
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKC
VNFNFNGLTGTGVLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCLIGAEHVNN
SYECDIPIGAGICASYQTQTNSPSRAGSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSPIEDLLFNKVTLADAGFIKQYGDC
LGDIAARDLICAQKFNGLTVLPPLLTDEMIAQYTSALLAGTITSGWTFGAGPALQIPFPM
QMAYRFNGIGVTQNVLYENQKLIANQFNSAIGKIQDSLSSTPSALGKLQDVVNQNAQAL
NTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESLID
L
QELGKYEQYIKWPWYIWLGFIAGLIAIVMVTIMLCCMTSCCSCLKGCCSCGSCCKFDED
DSEPVLKGVKLHYT
SEQ ID NO: 3, non-optimized DNA sequence encoding
SEQ ID NO: 2:
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGT
CTAACATAATAAGAGGCTGGATTTTTGGTACTACTTTAGATTCGAAGACCCAGTCCCT
ACTTATTGTTAATAACGCTACTAATGTTGTTATTAAAGTCTGTGAATTTCAATTTTGT
AATGATCCATTTTTGGGTGTTTATTACCACAAAAACAACAAAAGTTGGATGGAAAGT
GAGTTCAGAGTTTATTCTAGTGCGAATAATTGCACTTTTGAATATGTCTCTCAGCCTT
TTCTTATGGACCTTGAAGGAAAACAGGGTAATTTCAAAAATCTTAGGGAATTTGTGT
TTAAGAATATTGATGGTTATTTTAAAATATATTCTAAGCACACGCCTATTAATTTAGT
GCGTGATCTCCCTCAGGGTTTTTCGGCTTTAGAACCATTGGTAGATTTGCCAATAGGT
ATTAACATCACTAGGTTTCAAACTTTACTTGCTTTACATAGAAGTTATTTGACTCCTG
GTGATTCTTCTTCAGGTTGGACAGCTGGTGCTGCAGCTTATTATGTGGGTTATCTTCA
ACCTAGGACTTTTCTATTAAAATATAATGAAAATGGAACCATTACAGATGCTGTAGA
CTGTGCACTTGACCCTCTCTCAGAAACAAAGTGTACGTTGAAATCCTTCACTGTAGA
AAAAGGAATCTATCAAACTTCTAACTTTAGAGTCCAACCAACAGAATCTATTGTTAG
ATTTCCTAATATTACAAACTTGTGCCCTTTTGGTGAAGTTTTTAACGCCACCAGATTT
GCATCTGTTTATGCTTGGAACAGGAAGAGAATCAGCAACTGTGTTGCTGATTATTCT
GTCCTATATAATTCCGCATCATTTTCCACTTTTAAGTGTTATGGAGTGTCTCCTACTAA
ATTAAATGATCTCTGCTTTACTAATGTCTATGCAGATTCATTTGTAATTAGAGGTGAT
GAAGTCAGACAAATCGCTCCAGGGCAAACTGGAAAGATTGCTGATTATAATTATAAA
TTACCAGATGATTTTACAGGCTGCGTTATAGCTTGGAATTCTAACAATCTTGATTCTA
AGGTTGGTGGTAATTATAATTACCTGTATAGATTGTTTAGGAAGTCTAATCTCAAACC
TTTTGAGAGAGATATTTCAACTGAAATCTATCAGGCCGGTAGCACACCTTGTAATGG
TGTTGAAGGTTTTAATTGTTACTTTCCTTTACAATCATATGGTTTCCAACCCACTAATG
GTGTTGGTTACCAACCATACAGAGTAGTAGTACTTTCTTTTGAACTTCTACATGCACC
AGCAACTGTTTGTGGACCTAAAAAGTCTACTAATTTGGTTAAAAACAAATGTGTCAA
TTTCAACTTCAATGGTTTAACAGGCACAGGTGTTCTTACTGAGTCTAACAAAAAGTTT
CTGCCTTTCCAACAATTTGGCAGAGACATTGCTGACACTACTGATGCTGTCCGTGATC
CACAGACACTTGAGATTCTTGACATTACACCATGTTCTTTTGGTGGTGTCAGTGTTAT
AACACCAGGAACAAATACTTCTAACCAGGTTGCTGTTCTTTATCAGGGTGTTAACTG
CACAGAAGTCCCTGTTGCTATTCATGCAGATCAACTTACTCCTACTTGGCGTGTTTAT
TCTACAGGTTCTAATGTTTTTCAAACACGTGCAGGCTGTTTAATAGGGGCTGAACATG
TCAACAACTCATATGAGTGTGACATACCCATTGGTGCAGGTATATGCGCTAGTTATC
AGACTCAGACTAATTCTCCTTCGCGGGCAGGTAGTGTAGCTAGTCAATCCATCATTG
CCTACACTATGTCACTTGGTGCAGAAAATTCAGTTGCTTACTCTAATAACTCTATTGC
CATACCCACAAATTTTACTATTAGTGTTACCACAGAAATTCTACCAGTGTCTATGACC
AAGACATCAGTAGATTGTACAATGTACATTTGTGGTGATTCAACTGAATGCAGCAAT
CTTTTGTTGCAATATGGCAGTTTTTGTACACAATTAAACCGTGCTTTAACTGGAATAG
CTGTTGAACAAGACAAAAACACCCAAGAAGTTTTTGCACAAGTCAAACAAATTTACA
AAACACCACCAATTAAAGATTTTGGTGGTTTTAATTTTTCACAAATATTACCAGATCC
ATCAAAACCAAGCAAGAGGTCACCTATTGAAGATCTACTTTTCAACAAAGTGACACT
TGCAGATGCTGGCTTCATCAAACAATATGGTGATTGCCTTGGTGATATTGCTGCTAGA
GACCTCATTTGTGCACAAAAGTTTAACGGCCTTACTGTTTTGCCACCTTTGCTCACAG
ATGAAATGATTGCTCAATACACTTCTGCACTGTTAGCGGGTACAATCACTTCTGGTTG
GACCTTTGGTGCAGGTCCTGCATTACAAATACCATTTCCTATGCAAATGGCTTATAGG
TTTAATGGTATTGGAGTTACACAGAATGTTCTCTATGAGAACCAAAAATTGATTGCC
AACCAATTTAATAGTGCTATTGGCAAAATTCAAGACTCACTTTCTTCCACACCAAGTG
CACTTGGAAAACTTCAAGATGTGGTCAACCAAAATGCACAAGCTTTAAACACGCTTG
TTAAACAACTTAGCTCCAATTTTGGTGCAATTTCAAGTGTTTTAAATGATATCCTTTC
ACGTCTTGACCCACCTGAGGCTGAAGTGCAAATTGATAGGTTGATCACAGGCAGACT
TCAAAGTTTGCAGACATATGTGACTCAACAATTAATTAGAGCTGCAGAAATCAGAGC
TTCTGCTAATCTTGCTGCTACTAAAATGTCAGAGTGTGTACTTGGACAATCAAAAAG
AGTTGATTTTTGTGGAAAGGGCTATCATCTTATGTCCTTCCCTCAGTCAGCACCTCAT
GGTGTAGTCTTCTTGCATGTGACTTATGTCCCTGCACAAGAAAAGAACTTCACAACT
GCTCCTGCCATTTGTCATGATGGAAAAGCACACTTTCCTCGTGAAGGTGTCTTTGTTT
CAAATGGCACACACTGGTTTGTAACACAAAGGAATTTTTATGAACCACAAATCATTA
CTACAGACAACACATTTGTGTCTGGTAACTGTGATGTTGTAATAGGAATTGTCAACA
ACACAGTTTATGATCCTTTGCAACCTGAATTAGACTCATTCAAGGAGGAGTTAGATA
AATATTTTAAGAATCATACATCACCAGATGTTGATTTAGGTGACATCTCTGGCATTAA
TGCTTCAGTTGTAAACATTCAAAAAGAAATTGACCGCCTCAATGAGGTTGCCAAGAA
TTTAAATGAATCTCTCATCGATCTCCAAGAACTTGGAAAGTATGAGCAGTATATAAA
ATGGCCATGGTACATTTGGCTAGGTTTTATAGCTGGCTTGATTGCCATAGTAATGGTG
ACAATTATGCTTTGCTGTATGACCAGTTGCTGTAGTTGTCTCAAGGGCTGTTGTTCTT
GTGGATCCTGCTGCAAATTTGATGAAGACGACTCTGAGCCAGTGCTCAAAGGAGTCA
AATTACATTACACATAA
SEQ ID NO: 4, non-optimized RNA sequence encoding
SEQ ID NO: 2:
AUGUUUGUUUUUCUUGUUUUAUUGCCACUAGUCUCUAGUCAGUGUGUUAAUCUU
ACAACCAGAACUCAAUUACCCCCUGCAUACACUAAUUCUUUCACACGUGGUGUUU
AUUACCCUGACAAAGUUUUCAGAUCCUCAGUUUUACAUUCAACUCAGGACUUGUU
CUUACCUUUCUUUUCCAAUGUUACUUGGUUCCAUGCUAUACAUGUCUCUGGGACC
AAUGGUACUAAGAGGUUUGAUAACCCUGUCCUACCAUUUAAUGAUGGUGUUUAU
UUUGCUUCCACUGAGAAGUCUAACAUAAUAAGAGGCUGGAUUUUUGGUACUACUU
UAGAUUCGAAGACCCAGUCCCUACUUAUUGUUAAUAACGCUACUAAUGUUGUUAU
UAAAGUCUGUGAAUUUCAAUUUUGUAAUGAUCCAUUUUUGGGUGUUUAUUACCA
CAAAAACAACAAAAGUUGGAUGGAAAGUGAGUUCAGAGUUUAUUCUAGUGCGAA
UAAUUGCACUUUUGAAUAUGUCUCUCAGCCUUUUCUUAUGGACCUUGAAGGAAAA
CAGGGUAAUUUCAAAAAUCUUAGGGAAUUUGUGUUUAAGAAUAUUGAUGGUUAU
UUUAAAAUAUAUUCUAAGCACACGCCUAUUAAUUUAGUGCGUGAUCUCCCUCAGG
GUUUUUCGGCUUUAGAACCAUUGGUAGAUUUGCCAAUAGGUAUUAACAUCACUAG
GUUUCAAACUUUACUUGCUUUACAUAGAAGUUAUUUGACUCCUGGUGAUUCUUCU
UCAGGUUGGACAGCUGGUGCUGCAGCUUAUUAUGUGGGUUAUCUUCAACCUAGGA
CUUUUCUAUUAAAAUAUAAUGAAAAUGGAACCAUUACAGAUGCUGUAGACUGUG
CACUUGACCCUCUCUCAGAAACAAAGUGUACGUUGAAAUCCUUCACUGUAGAAAA
AGGAAUCUAUCAAACUUCUAACUUUAGAGUCCAACCAACAGAAUCUAUUGUUAGA
UUUCCUAAUAUUACAAACUUGUGCCCUUUUGGUGAAGUUUUUAACGCCACCAGAU
UUGCAUCUGUUUAUGCUUGGAACAGGAAGAGAAUCAGCAACUGUGUUGCUGAUU
AUUCUGUCCUAUAUAAUUCCGCAUCAUUUUCCACUUUUAAGUGUUAUGGAGUGUC
UCCUACUAAAUUAAAUGAUCUCUGCUUUACUAAUGUCUAUGCAGAUUCAUUUGUA
AUUAGAGGUGAUGAAGUCAGACAAAUCGCUCCAGGGCAAACUGGAAAGAUUGCUG
AUUAUAAUUAUAAAUUACCAGAUGAUUUUACAGGCUGCGUUAUAGCUUGGAAUU
CUAACAAUCUUGAUUCUAAGGUUGGUGGUAAUUAUAAUUACCUGUAUAGAUUGU
UUAGGAAGUCUAAUCUCAAACCUUUUGAGAGAGAUAUUUCAACUGAAAUCUAUCA
GGCCGGUAGCACACCUUGUAAUGGUGUUGAAGGUUUUAAUUGUUACUUUCCUUUA
CAAUCAUAUGGUUUCCAACCCACUAAUGGUGUUGGUUACCAACCAUACAGAGUAG
UAGUACUUUCUUUUGAACUUCUACAUGCACCAGCAACUGUUUGUGGACCUAAAAA
GUCUACUAAUUUGGUUAAAAACAAAUGUGUCAAUUUCAACUUCAAUGGUUUAAC
AGGCACAGGUGUUCUUACUGAGUCUAACAAAAAGUUUCUGCCUUUCCAACAAUUU
GGCAGAGACAUUGCUGACACUACUGAUGCUGUCCGUGAUCCACAGACACUUGAGA
UUCUUGACAUUACACCAUGUUCUUUUGGUGGUGUCAGUGUUAUAACACCAGGAAC
AAAUACUUCUAACCAGGUUGCUGUUCUUUAUCAGGGUGUUAACUGCACAGAAGUC
CCUGUUGCUAUUCAUGCAGAUCAACUUACUCCUACUUGGCGUGUUUAUUCUACAG
GUUCUAAUGUUUUUCAAACACGUGCAGGCUGUUUAAUAGGGGCUGAACAUGUCAA
CAACUCAUAUGAGUGUGACAUACCCAUUGGUGCAGGUAUAUGCGCUAGUUAUCAG
ACUCAGACUAAUUCUCCUUCGCGGGCAGGUAGUGUAGCUAGUCAAUCCAUCAUUG
CCUACACUAUGUCACUUGGUGCAGAAAAUUCAGUUGCUUACUCUAAUAACUCUAU
UGCCAUACCCACAAAUUUUACUAUUAGUGUUACCACAGAAAUUCUACCAGUGUCU
AUGACCAAGACAUCAGUAGAUUGUACAAUGUACAUUUGUGGUGAUUCAACUGAA
UGCAGCAAUCUUUUGUUGCAAUAUGGCAGUUUUUGUACACAAUUAAACCGUGCUU
UAACUGGAAUAGCUGUUGAACAAGACAAAAACACCCAAGAAGUUUUUGCACAAGU
CAAACAAAUUUACAAAACACCACCAAUUAAAGAUUUUGGUGGUUUUAAUUUUUCA
CAAAUAUUACCAGAUCCAUCAAAACCAAGCAAGAGGUCACCUAUUGAAGAUCUAC
UUUUCAACAAAGUGACACUUGCAGAUGCUGGCUUCAUCAAACAAUAUGGUGAUUG
CCUUGGUGAUAUUGCUGCUAGAGACCUCAUUUGUGCACAAAAGUUUAACGGCCUU
ACUGUUUUGCCACCUUUGCUCACAGAUGAAAUGAUUGCUCAAUACACUUCUGCAC
UGUUAGCGGGUACAAUCACUUCUGGUUGGACCUUUGGUGCAGGUCCUGCAUUACA
AAUACCAUUUCCUAUGCAAAUGGCUUAUAGGUUUAAUGGUAUUGGAGUUACACA
GAAUGUUCUCUAUGAGAACCAAAAAUUGAUUGCCAACCAAUUUAAUAGUGCUAUU
GGCAAAAUUCAAGACUCACUUUCUUCCACACCAAGUGCACUUGGAAAACUUCAAG
AUGUGGUCAACCAAAAUGCACAAGCUUUAAACACGCUUGUUAAACAACUUAGCUC
CAAUUUUGGUGCAAUUUCAAGUGUUUUAAAUGAUAUCCUUUCACGUCUUGACCCA
CCUGAGGCUGAAGUGCAAAUUGAUAGGUUGAUCACAGGCAGACUUCAAAGUUUGC
AGACAUAUGUGACUCAACAAUUAAUUAGAGCUGCAGAAAUCAGAGCUUCUGCUAA
UCUUGCUGCUACUAAAAUGUCAGAGUGUGUACUUGGACAAUCAAAAAGAGUUGA
UUUUUGUGGAAAGGGCUAUCAUCUUAUGUCCUUCCCUCAGUCAGCACCUCAUGGU
GUAGUCUUCUUGCAUGUGACUUAUGUCCCUGCACAAGAAAAGAACUUCACAACUG
CUCCUGCCAUUUGUCAUGAUGGAAAAGCACACUUUCCUCGUGAAGGUGUCUUUGU
UUCAAAUGGCACACACUGGUUUGUAACACAAAGGAAUUUUUAUGAACCACAAAUC
AUUACUACAGACAACACAUUUGUGUCUGGUAACUGUGAUGUUGUAAUAGGAAUU
GUCAACAACACAGUUUAUGAUCCUUUGCAACCUGAAUUAGACUCAUUCAAGGAGG
AGUUAGAUAAAUAUUUUAAGAAUCAUACAUCACCAGAUGUUGAUUUAGGUGACA
UCUCUGGCAUUAAUGCUUCAGUUGUAAACAUUCAAAAAGAAAUUGACCGCCUCAA
UGAGGUUGCCAAGAAUUUAAAUGAAUCUCUCAUCGAUCUCCAAGAACUUGGAAAG
UAUGAGCAGUAUAUAAAAUGGCCAUGGUACAUUUGGCUAGGUUUUAUAGCUGGC
UUGAUUGCCAUAGUAAUGGUGACAAUUAUGCUUUGCUGUAUGACCAGUUGCUGU
AGUUGUCUCAAGGGCUGUUGUUCUUGUGGAUCCUGCUGCAAAUUUGAUGAAGACG
ACUCUGAGCCAGUGCUCAAAGGAGUCAAAUUACAUUACACAUAA
SEQ ID NO: 5, amino acid sequence of S protein comprising
the following mutations: delta variant mutations of T19R,
T95I, G142D, E156Δ, F157Δ, R158G, L452R, T478K, D614G,
P681R, and D950N; S2P (K986P and V987P); and furin-like
cleavage site mutations (R682S and R685G):
MFVFLVLLPLVSSQCVNLRTRTQLPPAYTNSFTRGVYYPDKVFRSSVLHSTQDLFLPFFSN
VTWFHAIHVSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYRYRLFRKSNLKPFERDISTEIYQAGSKPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECDI
PIGAGICASYQTQTNSRSRAGSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQNVVNQNAQALNTLVKQLSS
NFGAISSVLNDILSRLDPPEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVK
LHYT.
SEQ ID NO: 6, amino acid sequence of S protein comprising
the following mutations: delta variant mutations of T19R,
T95I, G142D, E156Δ, F157Δ, R158G, L452R, T478K, D614G,
D950N; S2P (K986P and V987P); and furin-like cleavage site
mutations (R682S and R685G). It lacks the P681R mutation
compared to SEQ ID NO: 5:
MFVFLVLLPLVSSQCVNLRTRTQLPPAYTNSFTRGVYYPDKVFRSSVLHSTQDLFLPFFSN
VTWFHAIHVSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYRYRLFRKSNLKPFERDISTEIYQAGSKPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECDI
PIGAGICASYQTQTNSPSRAGSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQNVVNQNAQALNTLVKQLSS
NFGAISSVLNDILSRLDPPEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVK
LHYT.
SEQ ID NO: 7, amino acid sequence of S protein comprising
the following mutations: delta variant mutations of T19R,
T95I, G142D, E156Δ, F157Δ, R158G, L452R, T478K, D614G,
P681R, and D950N; S6P (F817P, A892P, A899P, A942P, K986P,
and V987P); and furin-like cleavage site mutations (R682S
and R685G). It further comprises the mutations of F817P,
A892P, A899P, and A942P, compared to SEQ ID NO: 5:
MFVFLVLLPLVSSQCVNLRTRTQLPPAYTNSFTRGVYYPDKVFRSSVLHSTQDLFLPFFSN
VTWFHAIHVSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYRYRLFRKSNLKPFERDISTEIYQAGSKPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECDI
PIGAGICASYQTQTNSRSRAGSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSPIEDLLFNKVTLADAGFIKQYGDCLGDIAARD
LICAQKFNGLTVLPPLLTDEMIAQYTSALLAGTITSGWTFGAGPALQIPFPMQMAYRFNGI
GVTQNVLYENQKLIANQFNSAIGKIQDSLSSTPSALGKLQNVVNQNAQALNTLVKQLSS
NFGAISSVLNDILSRLDPPEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVK
LHYT.
SEQ ID NO: 8, optimized DNA sequence encoding
SEQ ID NO: 7:
ATGTTCGTGTTCCTGGTGCTGCTGCCTCTGGTCAGCAGCCAGTGCGTGAACCTGAGA
ACAAGAACACAGCTTCCTCCAGCCTACACAAACTCTTTTACACGGGGCGTGTACTAT
CCTGACAAGGTGTTCCGGTCCAGCGTGCTGCACTCAACCCAAGACCTGTTCCTGCCCT
TCTTCAGCAACGTCACCTGGTTCCACGCCATCCACGTGTCTGGCACCAATGGCACAA
AGCGATTCGATAACCCCGTGCTGCCTTTCAACGACGGCGTGTACTTTGCCTCCATCGA
GAAGTCCAACATCATCCGGGGCTGGATCTTCGGGACCACACTGGATAGCAAGACCCA
GTCTCTGCTGATCGTAAACAACGCCACCAACGTGGTCATCAAGGTGTGCGAGTTCCA
GTTCTGCAACGACCCTTTCCTCGATGTGTACTACCACAAGAACAACAAGTCTTGGAT
GGAATCGGGCGTGTATAGCAGCGCCAACAACTGCACCTTCGAATACGTGAGCCAGCC
TTTCCTGATGGACCTGGAAGGCAAACAAGGCAATTTTAAGAACCTGAGAGAATTCGT
GTTCAAAAATATAGACGGCTATTTCAAGATCTACAGCAAGCACACCCCTATTAATCT
GGTGCGGGATCTGCCTCAGGGCTTCAGCGCCCTCGAACCTCTGGTGGACCTGCCAAT
CGGCATCAACATTACAAGATTCCAGACGCTGCTCGCTCTGCACAGATCTTACCTGAC
CCCTGGCGACAGCAGCAGCGGCTGGACCGCCGGCGCCGCCGCTTACTACGTGGGCTA
CCTGCAGCCTAGAACCTTTCTGCTGAAGTACAACGAGAACGGCACCATCACTGATGC
CGTGGATTGCGCCCTGGACCCTCTGTCCGAAACCAAATGTACACTGAAGTCTTTTACC
GTGGAAAAAGGAATCTACCAGACTTCCAACTTCCGGGTGCAGCCGACCGAGAGCAT
CGTGCGGTTCCCTAACATCACAAACCTGTGCCCCTTTGGCGAGGTGTTCAACGCCAC
AAGATTTGCTAGCGTGTACGCCTGGAATAGAAAGAGAATCAGCAACTGCGTGGCCG
ATTACAGCGTGCTGTACAATAGCGCCTCTTTCAGCACCTTCAAATGCTACGGCGTGA
GCCCCACCAAGCTGAACGATCTGTGTTTTACAAACGTGTATGCCGACTCATTCGTAAT
CAGGGGCGATGAGGTGAGACAGATCGCTCCTGGACAGACAGGCAAAATCGCGGACT
ACAACTATAAGCTGCCTGATGACTTCACAGGATGTGTGATCGCATGGAACTCCAATA
ACCTCGACAGCAAGGTGGGCGGAAATTACAATTACCGCTACAGACTGTTTAGAAAG
AGCAATCTGAAACCTTTCGAGAGAGACATCAGCACAGAGATCTACCAGGCCGGCAG
CAAGCCCTGTAACGGCGTCGAGGGCTTCAACTGCTACTTCCCCCTGCAGAGCTACGG
CTTCCAGCCTACCAACGGCGTGGGATACCAGCCTTACAGAGTGGTGGTGCTGAGCTT
CGAGCTGCTGCATGCTCCTGCTACAGTGTGTGGTCCTAAGAAGAGCACCAACCTGGT
TAAGAACAAGTGCGTGAATTTTAACTTCAATGGACTGACCGGAACCGGCGTGCTGAC
CGAAAGCAACAAGAAATTCCTGCCTTTTCAGCAGTTTGGCAGAGACATCGCCGACAC
CACCGACGCCGTGAGAGATCCACAAACCCTGGAAATCCTGGACATCACACCTTGCTC
ATTTGGAGGGGTGTCGGTGATCACACCTGGCACCAACACCAGCAACCAGGTGGCCGT
GCTGTACCAGGGAGTGAATTGTACCGAGGTCCCCGTGGCCATTCACGCCGACCAGCT
GACCCCTACCTGGCGGGTGTACTCCACCGGCTCTAACGTATTCCAGACCAGAGCCGG
CTGTCTGATCGGCGCAGAACACGTGAACAATAGCTACGAGTGCGACATCCCTATCGG
AGCCGGGATCTGCGCTAGCTACCAGACCCAGACAAACTCCAGAAGCAGAGCCGGAA
GCGTGGCCAGCCAGTCTATCATCGCCTACACCATGAGCCTGGGCGCCGAAAACAGCG
TTGCCTACAGCAACAATTCTATCGCCATCCCTACAAACTTCACCATCTCCGTGACCAC
CGAGATCCTGCCTGTCAGCATGACAAAGACCAGCGTAGACTGCACAATGTACATCTG
CGGAGATTCCACCGAGTGTAGTAACCTCCTGCTGCAATACGGATCTTTCTGTACTCAG
CTGAACAGAGCCCTGACCGGCATCGCCGTTGAACAGGACAAGAACACCCAGGAGGT
TTTCGCCCAGGTTAAGCAGATCTACAAAACCCCTCCTATCAAGGACTTCGGAGGCTT
TAACTTCTCCCAGATCCTGCCCGACCCCAGCAAGCCCAGCAAGCGGAGCCCCATCGA
GGACCTGCTGTTCAACAAGGTGACCCTGGCCGACGCCGGCTTCATCAAACAGTACGG
CGATTGCCTGGGAGACATCGCCGCTAGAGATCTAATTTGCGCCCAAAAGTTTAACGG
CCTGACAGTGCTGCCTCCACTGCTGACAGACGAGATGATCGCCCAGTACACATCTGC
CCTGCTGGCTGGTACCATCACATCTGGCTGGACCTTTGGCGCCGGCCCCGCCCTCCAG
ATCCCTTTCCCCATGCAGATGGCCTACCGGTTCAACGGCATCGGCGTGACCCAGAAC
GTGCTGTACGAAAACCAGAAACTGATCGCCAACCAGTTCAATAGCGCGATCGGCAA
AATCCAGGATAGCCTCAGCTCTACACCCAGCGCTCTTGGCAAGCTGCAAAACGTGGT
GAACCAGAATGCCCAGGCCCTTAACACCCTGGTGAAGCAGCTATCCTCTAATTTCGG
TGCCATCAGCAGCGTGCTGAATGATATCCTGAGCAGACTGGACCCCCCTGAGGCCGA
AGTGCAGATCGACAGACTGATCACCGGAAGACTGCAGAGCCTGCAAACCTACGTGA
CCCAGCAACTGATCCGGGCCGCAGAAATCCGGGCCTCCGCTAACCTGGCCGCTACCA
AGATGAGCGAGTGCGTGCTGGGTCAAAGCAAGCGCGTGGACTTCTGTGGAAAAGGC
TACCACCTGATGAGCTTCCCTCAGAGCGCTCCACACGGCGTGGTGTTCCTGCATGTG
ACTTACGTGCCTGCCCAGGAAAAGAACTTCACCACCGCCCCTGCCATTTGTCACGAC
GGCAAGGCCCACTTCCCCCGGGAAGGCGTGTTTGTGTCTAACGGAACACACTGGTTT
GTGACTCAAAGAAACTTCTACGAGCCACAGATCATCACCACAGATAACACCTTCGTC
AGCGGCAACTGCGACGTGGTGATCGGCATCGTGAACAATACTGTGTACGACCCCCTG
CAGCCAGAGCTCGATTCTTTCAAAGAGGAACTGGATAAGTACTTCAAGAACCACACA
TCCCCCGACGTCGACCTGGGCGATATCAGCGGCATTAACGCCAGCGTGGTGAACATC
CAGAAGGAAATCGATAGACTGAACGAGGTGGCAAAGAACCTGAATGAGTCCCTGAT
TGACCTGCAAGAGCTCGGGAAGTACGAGCAGTATATCAAGTGGCCTTGGTACATCTG
GCTGGGCTTCATCGCGGGCCTGATCGCCATCGTTATGGTGACGATCATGCTGTGCTGC
ATGACCAGTTGCTGTAGCTGCCTGAAGGGCTGCTGCAGCTGCGGCAGCTGTTGCAAG
TTCGACGAGGACGACAGCGAGCCTGTGCTGAAGGGCGTTAAGCTGCACTACACCTGA
SEQ ID NO: 9, optimized RNA sequence encoding
SEQ ID NO: 7:
AUGUUCGUGUUCCUGGUGCUGCUGCCUCUGGUCAGCAGCCAGUGCGUGAACCUGA
GAACAAGAACACAGCUUCCUCCAGCCUACACAAACUCUUUUACACGGGGCGUGUA
CUAUCCUGACAAGGUGUUCCGGUCCAGCGUGCUGCACUCAACCCAAGACCUGUUC
CUGCCCUUCUUCAGCAACGUCACCUGGUUCCACGCCAUCCACGUGUCUGGCACCAA
UGGCACAAAGCGAUUCGAUAACCCCGUGCUGCCUUUCAACGACGGCGUGUACUUU
GCCUCCAUCGAGAAGUCCAACAUCAUCCGGGGCUGGAUCUUCGGGACCACACUGG
AUAGCAAGACCCAGUCUCUGCUGAUCGUAAACAACGCCACCAACGUGGUCAUCAA
GGUGUGCGAGUUCCAGUUCUGCAACGACCCUUUCCUCGAUGUGUACUACCACAAG
AACAACAAGUCUUGGAUGGAAUCGGGCGUGUAUAGCAGCGCCAACAACUGCACCU
UCGAAUACGUGAGCCAGCCUUUCCUGAUGGACCUGGAAGGCAAACAAGGCAAUUU
UAAGAACCUGAGAGAAUUCGUGUUCAAAAAUAUAGACGGCUAUUUCAAGAUCUAC
AGCAAGCACACCCCUAUUAAUCUGGUGCGGGAUCUGCCUCAGGGCUUCAGCGCCC
UCGAACCUCUGGUGGACCUGCCAAUCGGCAUCAACAUUACAAGAUUCCAGACGCU
GCUCGCUCUGCACAGAUCUUACCUGACCCCUGGCGACAGCAGCAGCGGCUGGACC
GCCGGCGCCGCCGCUUACUACGUGGGCUACCUGCAGCCUAGAACCUUUCUGCUGA
AGUACAACGAGAACGGCACCAUCACUGAUGCCGUGGAUUGCGCCCUGGACCCUCU
GUCCGAAACCAAAUGUACACUGAAGUCUUUUACCGUGGAAAAAGGAAUCUACCAG
ACUUCCAACUUCCGGGUGCAGCCGACCGAGAGCAUCGUGCGGUUCCCUAACAUCA
CAAACCUGUGCCCCUUUGGCGAGGUGUUCAACGCCACAAGAUUUGCUAGCGUGUA
CGCCUGGAAUAGAAAGAGAAUCAGCAACUGCGUGGCCGAUUACAGCGUGCUGUAC
AAUAGCGCCUCUUUCAGCACCUUCAAAUGCUACGGCGUGAGCCCCACCAAGCUGA
ACGAUCUGUGUUUUACAAACGUGUAUGCCGACUCAUUCGUAAUCAGGGGCGAUGA
GGUGAGACAGAUCGCUCCUGGACAGACAGGCAAAAUCGCGGACUACAACUAUAAG
CUGCCUGAUGACUUCACAGGAUGUGUGAUCGCAUGGAACUCCAAUAACCUCGACA
GCAAGGUGGGCGGAAAUUACAAUUACCGCUACAGACUGUUUAGAAAGAGCAAUCU
GAAACCUUUCGAGAGAGACAUCAGCACAGAGAUCUACCAGGCCGGCAGCAAGCCC
UGUAACGGCGUCGAGGGCUUCAACUGCUACUUCCCCCUGCAGAGCUACGGCUUCC
AGCCUACCAACGGCGUGGGAUACCAGCCUUACAGAGUGGUGGUGCUGAGCUUCGA
GCUGCUGCAUGCUCCUGCUACAGUGUGUGGUCCUAAGAAGAGCACCAACCUGGUU
AAGAACAAGUGCGUGAAUUUUAACUUCAAUGGACUGACCGGAACCGGCGUGCUGA
CCGAAAGCAACAAGAAAUUCCUGCCUUUUCAGCAGUUUGGCAGAGACAUCGCCGA
CACCACCGACGCCGUGAGAGAUCCACAAACCCUGGAAAUCCUGGACAUCACACCU
UGCUCAUUUGGAGGGGUGUCGGUGAUCACACCUGGCACCAACACCAGCAACCAGG
UGGCCGUGCUGUACCAGGGAGUGAAUUGUACCGAGGUCCCCGUGGCCAUUCACGC
CGACCAGCUGACCCCUACCUGGCGGGUGUACUCCACCGGCUCUAACGUAUUCCAG
ACCAGAGCCGGCUGUCUGAUCGGCGCAGAACACGUGAACAAUAGCUACGAGUGCG
ACAUCCCUAUCGGAGCCGGGAUCUGCGCUAGCUACCAGACCCAGACAAACUCCAG
AAGCAGAGCCGGAAGCGUGGCCAGCCAGUCUAUCAUCGCCUACACCAUGAGCCUG
GGCGCCGAAAACAGCGUUGCCUACAGCAACAAUUCUAUCGCCAUCCCUACAAACU
UCACCAUCUCCGUGACCACCGAGAUCCUGCCUGUCAGCAUGACAAAGACCAGCGU
AGACUGCACAAUGUACAUCUGCGGAGAUUCCACCGAGUGUAGUAACCUCCUGCUG
CAAUACGGAUCUUUCUGUACUCAGCUGAACAGAGCCCUGACCGGCAUCGCCGUUG
AACAGGACAAGAACACCCAGGAGGUUUUCGCCCAGGUUAAGCAGAUCUACAAAAC
CCCUCCUAUCAAGGACUUCGGAGGCUUUAACUUCUCCCAGAUCCUGCCCGACCCCA
GCAAGCCCAGCAAGCGGAGCCCCAUCGAGGACCUGCUGUUCAACAAGGUGACCCU
GGCCGACGCCGGCUUCAUCAAACAGUACGGCGAUUGCCUGGGAGACAUCGCCGCU
AGAGAUCUAAUUUGCGCCCAAAAGUUUAACGGCCUGACAGUGCUGCCUCCACUGC
UGACAGACGAGAUGAUCGCCCAGUACACAUCUGCCCUGCUGGCUGGUACCAUCAC
AUCUGGCUGGACCUUUGGCGCCGGCCCCGCCCUCCAGAUCCCUUUCCCCAUGCAGA
UGGCCUACCGGUUCAACGGCAUCGGCGUGACCCAGAACGUGCUGUACGAAAACCA
GAAACUGAUCGCCAACCAGUUCAAUAGCGCGAUCGGCAAAAUCCAGGAUAGCCUC
AGCUCUACACCCAGCGCUCUUGGCAAGCUGCAAAACGUGGUGAACCAGAAUGCCC
AGGCCCUUAACACCCUGGUGAAGCAGCUAUCCUCUAAUUUCGGUGCCAUCAGCAG
CGUGCUGAAUGAUAUCCUGAGCAGACUGGACCCCCCUGAGGCCGAAGUGCAGAUC
GACAGACUGAUCACCGGAAGACUGCAGAGCCUGCAAACCUACGUGACCCAGCAAC
UGAUCCGGGCCGCAGAAAUCCGGGCCUCCGCUAACCUGGCCGCUACCAAGAUGAG
CGAGUGCGUGCUGGGUCAAAGCAAGCGCGUGGACUUCUGUGGAAAAGGCUACCAC
CUGAUGAGCUUCCCUCAGAGCGCUCCACACGGCGUGGUGUUCCUGCAUGUGACUU
ACGUGCCUGCCCAGGAAAAGAACUUCACCACCGCCCCUGCCAUUUGUCACGACGGC
AAGGCCCACUUCCCCCGGGAAGGCGUGUUUGUGUCUAACGGAACACACUGGUUUG
UGACUCAAAGAAACUUCUACGAGCCACAGAUCAUCACCACAGAUAACACCUUCGU
CAGCGGCAACUGCGACGUGGUGAUCGGCAUCGUGAACAAUACUGUGUACGACCCC
CUGCAGCCAGAGCUCGAUUCUUUCAAAGAGGAACUGGAUAAGUACUUCAAGAACC
ACACAUCCCCCGACGUCGACCUGGGCGAUAUCAGCGGCAUUAACGCCAGCGUGGU
GAACAUCCAGAAGGAAAUCGAUAGACUGAACGAGGUGGCAAAGAACCUGAAUGAG
UCCCUGAUUGACCUGCAAGAGCUCGGGAAGUACGAGCAGUAUAUCAAGUGGCCUU
GGUACAUCUGGCUGGGCUUCAUCGCGGGCCUGAUCGCCAUCGUUAUGGUGACGAU
CAUGCUGUGCUGCAUGACCAGUUGCUGUAGCUGCCUGAAGGGCUGCUGCAGCUGC
GGCAGCUGUUGCAAGUUCGACGAGGACGACAGCGAGCCUGUGCUGAAGGGCGUUA
AGCUGCACUACACCUGA
SEQ ID NO: 10, amino acid sequence of S protein comprising
the following mutations: delta variant mutations of T19R,
T95I, G142D, E156Δ, F157Δ, R158G, L452R, T478K, D614G, and
D950N; S6P (F817P, A892P, A899P, A942P, K986P, and V987P);
and furin-like cleavage site mutations (R682S and R685G).
It lacks the P681R mutation compared to SEQ ID NO: 7:
MFVFLVLLPLVSSQCVNLRTRTQLPPAYTNSFTRGVYYPDKVFRSSVLHSTQDLFLPFFSN
VTWFHAIHVSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYRYRLFRKSNLKPFERDISTEIYQAGSKPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECDI
PIGAGICASYQTQTNSPSRAGSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSPIEDLLFNKVTLADAGFIKQYGDCLGDIAARD
LICAQKFNGLTVLPPLLTDEMIAQYTSALLAGTITSGWTFGAGPALQIPFPMQMAYRFNGI
GVTQNVLYENQKLIANQFNSAIGKIQDSLSSTPSALGKLQNVVNQNAQALNTLVKQLSS
NFGAISSVLNDILSRLDPPEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVK
LHYT.
SEQ ID NO: 11, amino acid sequence of S protein comprising
the following mutations: delta variant mutations of T19R,
V70F, T95I, G142D, E156Δ, F157Δ, R158G, A222V, W258L, K417N,
L452R, T478K, D614G, P681R, and D950N; S6P (F817P, A892P,
A899P, A942P, K986P, and V987P); and furin-like cleavage
site mutations (R682S and R685G):
MFVFLVLLPLVSSQCVNLRTRTQLPPAYTNSFTRGVYYPDKVFRSSVLHSTQDLFLPFFSN
VTWFHAIHFSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSVLEPLVDLPIGINITRFQTLLAL
HRSYLTPGDSSSGLTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGNIADYNYK
LPDDFTGCVIAWNSNNLDSKVGGNYNYRYRLFRKSNLKPFERDISTEIYQAGSKPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEHVNNSYECDI
PIGAGICASYQTQTNSRSRAGSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSPIEDLLFNKVTLADAGFIKQYGDCLGDIAARD
LICAQKFNGLTVLPPLLTDEMIAQYTSALLAGTITSGWTFGAGPALQIPFPMQMAYRFNGI
GVTQNVLYENQKLIANQFNSAIGKIQDSLSSTPSALGKLQNVVNQNAQALNTLVKQLSS
NFGAISSVLNDILSRLDPPEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVK
LHYT.
SEQ ID NO: 12, optimized DNA sequence encoding
SEQ ID NO: 11:
ATGTTCGTGTTCCTGGTGCTGCTGCCTCTGGTCAGCAGCCAGTGCGTGAACCTGAGA
ACAAGAACACAGCTTCCTCCAGCCTACACAAACTCTTTTACACGGGGCGTGTACTAT
CCTGACAAGGTGTTCCGGTCCAGCGTGCTGCACTCAACCCAAGACCTGTTCCTGCCCT
TCTTCAGCAACGTCACCTGGTTCCACGCCATCCACTTCTCTGGCACCAATGGCACAAA
GCGATTCGATAACCCCGTGCTGCCTTTCAACGACGGCGTGTACTTTGCCTCCATCGAG
AAGTCCAACATCATCCGGGGCTGGATCTTCGGGACCACACTGGATAGCAAGACCCAG
TCTCTGCTGATCGTAAACAACGCCACCAACGTGGTCATCAAGGTGTGCGAGTTCCAG
TTCTGCAACGACCCTTTCCTCGATGTGTACTACCACAAGAACAACAAGTCTTGGATG
GAATCGGGCGTGTATAGCAGCGCCAACAACTGCACCTTCGAATACGTGAGCCAGCCT
TTCCTGATGGACCTGGAAGGCAAACAAGGCAATTTTAAGAACCTGAGAGAATTCGTG
TTCAAAAATATAGACGGCTATTTCAAGATCTACAGCAAGCACACCCCTATTAATCTG
GTGCGGGATCTGCCTCAGGGCTTCAGCGTCCTCGAACCTCTGGTGGACCTGCCAATC
GGCATCAACATTACAAGATTCCAGACGCTGCTCGCTCTGCACAGATCTTACCTGACC
CCTGGCGACAGCAGCAGCGGCCTGACCGCCGGCGCCGCCGCTTACTACGTGGGCTAC
CTGCAGCCTAGAACCTTTCTGCTGAAGTACAACGAGAACGGCACCATCACTGATGCC
GTGGATTGCGCCCTGGACCCTCTGTCCGAAACCAAATGTACACTGAAGTCTTTTACC
GTGGAAAAAGGAATCTACCAGACTTCCAACTTCCGGGTGCAGCCGACCGAGAGCAT
CGTGCGGTTCCCTAACATCACAAACCTGTGCCCCTTTGGCGAGGTGTTCAACGCCAC
AAGATTTGCTAGCGTGTACGCCTGGAATAGAAAGAGAATCAGCAACTGCGTGGCCG
ATTACAGCGTGCTGTACAATAGCGCCTCTTTCAGCACCTTCAAATGCTACGGCGTGA
GCCCCACCAAGCTGAACGATCTGTGTTTTACAAACGTGTATGCCGACTCATTCGTAAT
CAGGGGCGATGAGGTGAGACAGATCGCTCCTGGACAGACAGGCAAcATCGCGGACT
ACAACTATAAGCTGCCTGATGACTTCACAGGATGTGTGATCGCATGGAACTCCAATA
ACCTCGACAGCAAGGTGGGCGGAAATTACAATTACCGCTACAGACTGTTTAGAAAG
AGCAATCTGAAACCTTTCGAGAGAGACATCAGCACAGAGATCTACCAGGCCGGCAG
CAAGCCCTGTAACGGCGTCGAGGGCTTCAACTGCTACTTCCCCCTGCAGAGCTACGG
CTTCCAGCCTACCAACGGCGTGGGATACCAGCCTTACAGAGTGGTGGTGCTGAGCTT
CGAGCTGCTGCATGCTCCTGCTACAGTGTGTGGTCCTAAGAAGAGCACCAACCTGGT
TAAGAACAAGTGCGTGAATTTTAACTTCAATGGACTGACCGGAACCGGCGTGCTGAC
CGAAAGCAACAAGAAATTCCTGCCTTTTCAGCAGTTTGGCAGAGACATCGCCGACAC
CACCGACGCCGTGAGAGATCCACAAACCCTGGAAATCCTGGACATCACACCTTGCTC
ATTTGGAGGGGTGTCGGTGATCACACCTGGCACCAACACCAGCAACCAGGTGGCCGT
GCTGTACCAGGGAGTGAATTGTACCGAGGTCCCCGTGGCCATTCACGCCGACCAGCT
GACCCCTACCTGGCGGGTGTACTCCACCGGCTCTAACGTATTCCAGACCAGAGCCGG
CTGTCTGATCGGCGCAGAACACGTGAACAATAGCTACGAGTGCGACATCCCTATCGG
AGCCGGGATCTGCGCTAGCTACCAGACCCAGACAAACTCCAGAAGCAGAGCCGGAA
GCGTGGCCAGCCAGTCTATCATCGCCTACACCATGAGCCTGGGCGCCGAAAACAGCG
TTGCCTACAGCAACAATTCTATCGCCATCCCTACAAACTTCACCATCTCCGTGACCAC
CGAGATCCTGCCTGTCAGCATGACAAAGACCAGCGTAGACTGCACAATGTACATCTG
CGGAGATTCCACCGAGTGTAGTAACCTCCTGCTGCAATACGGATCTTTCTGTACTCAG
CTGAACAGAGCCCTGACCGGCATCGCCGTTGAACAGGACAAGAACACCCAGGAGGT
TTTCGCCCAGGTTAAGCAGATCTACAAAACCCCTCCTATCAAGGACTTCGGAGGCTT
TAACTTCTCCCAGATCCTGCCCGACCCCAGCAAGCCCAGCAAGCGGAGCCCCATCGA
GGACCTGCTGTTCAACAAGGTGACCCTGGCCGACGCCGGCTTCATCAAACAGTACGG
CGATTGCCTGGGAGACATCGCCGCTAGAGATCTAATTTGCGCCCAAAAGTTTAACGG
CCTGACAGTGCTGCCTCCACTGCTGACAGACGAGATGATCGCCCAGTACACATCTGC
CCTGCTGGCTGGTACCATCACATCTGGCTGGACCTTTGGCGCCGGCCCCGCCCTCCAG
ATCCCTTTCCCCATGCAGATGGCCTACCGGTTCAACGGCATCGGCGTGACCCAGAAC
GTGCTGTACGAAAACCAGAAACTGATCGCCAACCAGTTCAATAGCGCGATCGGCAA
AATCCAGGATAGCCTCAGCTCTACACCCAGCGCTCTTGGCAAGCTGCAAAACGTGGT
GAACCAGAATGCCCAGGCCCTTAACACCCTGGTGAAGCAGCTATCCTCTAATTTCGG
TGCCATCAGCAGCGTGCTGAATGATATCCTGAGCAGACTGGACCCCCCTGAGGCCGA
AGTGCAGATCGACAGACTGATCACCGGAAGACTGCAGAGCCTGCAAACCTACGTGA
CCCAGCAACTGATCCGGGCCGCAGAAATCCGGGCCTCCGCTAACCTGGCCGCTACCA
AGATGAGCGAGTGCGTGCTGGGTCAAAGCAAGCGCGTGGACTTCTGTGGAAAAGGC
TACCACCTGATGAGCTTCCCTCAGAGCGCTCCACACGGCGTGGTGTTCCTGCATGTG
ACTTACGTGCCTGCCCAGGAAAAGAACTTCACCACCGCCCCTGCCATTTGTCACGAC
GGCAAGGCCCACTTCCCCCGGGAAGGCGTGTTTGTGTCTAACGGAACACACTGGTTT
GTGACTCAAAGAAACTTCTACGAGCCACAGATCATCACCACAGATAACACCTTCGTC
AGCGGCAACTGCGACGTGGTGATCGGCATCGTGAACAATACTGTGTACGACCCCCTG
CAGCCAGAGCTCGATTCTTTCAAAGAGGAACTGGATAAGTACTTCAAGAACCACACA
TCCCCCGACGTCGACCTGGGCGATATCAGCGGCATTAACGCCAGCGTGGTGAACATC
CAGAAGGAAATCGATAGACTGAACGAGGTGGCAAAGAACCTGAATGAGTCCCTGAT
TGACCTGCAAGAGCTCGGGAAGTACGAGCAGTATATCAAGTGGCCTTGGTACATCTG
GCTGGGCTTCATCGCGGGCCTGATCGCCATCGTTATGGTGACGATCATGCTGTGCTGC
ATGACCAGTTGCTGTAGCTGCCTGAAGGGCTGCTGCAGCTGCGGCAGCTGTTGCAAG
TTCGACGAGGACGACAGCGAGCCTGTGCTGAAGGGCGTTAAGCTGCACTACACCTGA
SEQ ID NO: 13, optimized RNA sequence encoding
SEQ ID NO: 11:
AUGUUCGUGUUCCUGGUGCUGCUGCCUCUGGUCAGCAGCCAGUGCGUGAACCUGA
GAACAAGAACACAGCUUCCUCCAGCCUACACAAACUCUUUUACACGGGGCGUGUA
CUAUCCUGACAAGGUGUUCCGGUCCAGCGUGCUGCACUCAACCCAAGACCUGUUC
CUGCCCUUCUUCAGCAACGUCACCUGGUUCCACGCCAUCCACUUCUCUGGCACCAA
UGGCACAAAGCGAUUCGAUAACCCCGUGCUGCCUUUCAACGACGGCGUGUACUUU
GCCUCCAUCGAGAAGUCCAACAUCAUCCGGGGCUGGAUCUUCGGGACCACACUGG
AUAGCAAGACCCAGUCUCUGCUGAUCGUAAACAACGCCACCAACGUGGUCAUCAA
GGUGUGCGAGUUCCAGUUCUGCAACGACCCUUUCCUCGAUGUGUACUACCACAAG
AACAACAAGUCUUGGAUGGAAUCGGGCGUGUAUAGCAGCGCCAACAACUGCACCU
UCGAAUACGUGAGCCAGCCUUUCCUGAUGGACCUGGAAGGCAAACAAGGCAAUUU
UAAGAACCUGAGAGAAUUCGUGUUCAAAAAUAUAGACGGCUAUUUCAAGAUCUAC
AGCAAGCACACCCCUAUUAAUCUGGUGCGGGAUCUGCCUCAGGGCUUCAGCGUCC
UCGAACCUCUGGUGGACCUGCCAAUCGGCAUCAACAUUACAAGAUUCCAGACGCU
GCUCGCUCUGCACAGAUCUUACCUGACCCCUGGCGACAGCAGCAGCGGCCUGACCG
CCGGCGCCGCCGCUUACUACGUGGGCUACCUGCAGCCUAGAACCUUUCUGCUGAA
GUACAACGAGAACGGCACCAUCACUGAUGCCGUGGAUUGCGCCCUGGACCCUCUG
UCCGAAACCAAAUGUACACUGAAGUCUUUUACCGUGGAAAAAGGAAUCUACCAGA
CUUCCAACUUCCGGGUGCAGCCGACCGAGAGCAUCGUGCGGUUCCCUAACAUCAC
AAACCUGUGCCCCUUUGGCGAGGUGUUCAACGCCACAAGAUUUGCUAGCGUGUAC
GCCUGGAAUAGAAAGAGAAUCAGCAACUGCGUGGCCGAUUACAGCGUGCUGUACA
AUAGCGCCUCUUUCAGCACCUUCAAAUGCUACGGCGUGAGCCCCACCAAGCUGAA
CGAUCUGUGUUUUACAAACGUGUAUGCCGACUCAUUCGUAAUCAGGGGCGAUGAG
GUGAGACAGAUCGCUCCUGGACAGACAGGCAACAUCGCGGACUACAACUAUAAGC
UGCCUGAUGACUUCACAGGAUGUGUGAUCGCAUGGAACUCCAAUAACCUCGACAG
CAAGGUGGGCGGAAAUUACAAUUACCGCUACAGACUGUUUAGAAAGAGCAAUCUG
AAACCUUUCGAGAGAGACAUCAGCACAGAGAUCUACCAGGCCGGCAGCAAGCCCU
GUAACGGCGUCGAGGGCUUCAACUGCUACUUCCCCCUGCAGAGCUACGGCUUCCA
GCCUACCAACGGCGUGGGAUACCAGCCUUACAGAGUGGUGGUGCUGAGCUUCGAG
CUGCUGCAUGCUCCUGCUACAGUGUGUGGUCCUAAGAAGAGCACCAACCUGGUUA
AGAACAAGUGCGUGAAUUUUAACUUCAAUGGACUGACCGGAACCGGCGUGCUGAC
CGAAAGCAACAAGAAAUUCCUGCCUUUUCAGCAGUUUGGCAGAGACAUCGCCGAC
ACCACCGACGCCGUGAGAGAUCCACAAACCCUGGAAAUCCUGGACAUCACACCUU
GCUCAUUUGGAGGGGUGUCGGUGAUCACACCUGGCACCAACACCAGCAACCAGGU
GGCCGUGCUGUACCAGGGAGUGAAUUGUACCGAGGUCCCCGUGGCCAUUCACGCC
GACCAGCUGACCCCUACCUGGCGGGUGUACUCCACCGGCUCUAACGUAUUCCAGA
CCAGAGCCGGCUGUCUGAUCGGCGCAGAACACGUGAACAAUAGCUACGAGUGCGA
CAUCCCUAUCGGAGCCGGGAUCUGCGCUAGCUACCAGACCCAGACAAACUCCAGA
AGCAGAGCCGGAAGCGUGGCCAGCCAGUCUAUCAUCGCCUACACCAUGAGCCUGG
GCGCCGAAAACAGCGUUGCCUACAGCAACAAUUCUAUCGCCAUCCCUACAAACUU
CACCAUCUCCGUGACCACCGAGAUCCUGCCUGUCAGCAUGACAAAGACCAGCGUA
GACUGCACAAUGUACAUCUGCGGAGAUUCCACCGAGUGUAGUAACCUCCUGCUGC
AAUACGGAUCUUUCUGUACUCAGCUGAACAGAGCCCUGACCGGCAUCGCCGUUGA
ACAGGACAAGAACACCCAGGAGGUUUUCGCCCAGGUUAAGCAGAUCUACAAAACC
CCUCCUAUCAAGGACUUCGGAGGCUUUAACUUCUCCCAGAUCCUGCCCGACCCCAG
CAAGCCCAGCAAGCGGAGCCCCAUCGAGGACCUGCUGUUCAACAAGGUGACCCUG
GCCGACGCCGGCUUCAUCAAACAGUACGGCGAUUGCCUGGGAGACAUCGCCGCUA
GAGAUCUAAUUUGCGCCCAAAAGUUUAACGGCCUGACAGUGCUGCCUCCACUGCU
GACAGACGAGAUGAUCGCCCAGUACACAUCUGCCCUGCUGGCUGGUACCAUCACA
UCUGGCUGGACCUUUGGCGCCGGCCCCGCCCUCCAGAUCCCUUUCCCCAUGCAGAU
GGCCUACCGGUUCAACGGCAUCGGCGUGACCCAGAACGUGCUGUACGAAAACCAG
AAACUGAUCGCCAACCAGUUCAAUAGCGCGAUCGGCAAAAUCCAGGAUAGCCUCA
GCUCUACACCCAGCGCUCUUGGCAAGCUGCAAAACGUGGUGAACCAGAAUGCCCA
GGCCCUUAACACCCUGGUGAAGCAGCUAUCCUCUAAUUUCGGUGCCAUCAGCAGC
GUGCUGAAUGAUAUCCUGAGCAGACUGGACCCCCCUGAGGCCGAAGUGCAGAUCG
ACAGACUGAUCACCGGAAGACUGCAGAGCCUGCAAACCUACGUGACCCAGCAACU
GAUCCGGGCCGCAGAAAUCCGGGCCUCCGCUAACCUGGCCGCUACCAAGAUGAGC
GAGUGCGUGCUGGGUCAAAGCAAGCGCGUGGACUUCUGUGGAAAAGGCUACCACC
UGAUGAGCUUCCCUCAGAGCGCUCCACACGGCGUGGUGUUCCUGCAUGUGACUUA
CGUGCCUGCCCAGGAAAAGAACUUCACCACCGCCCCUGCCAUUUGUCACGACGGCA
AGGCCCACUUCCCCCGGGAAGGCGUGUUUGUGUCUAACGGAACACACUGGUUUGU
GACUCAAAGAAACUUCUACGAGCCACAGAUCAUCACCACAGAUAACACCUUCGUC
AGCGGCAACUGCGACGUGGUGAUCGGCAUCGUGAACAAUACUGUGUACGACCCCC
UGCAGCCAGAGCUCGAUUCUUUCAAAGAGGAACUGGAUAAGUACUUCAAGAACCA
CACAUCCCCCGACGUCGACCUGGGCGAUAUCAGCGGCAUUAACGCCAGCGUGGUG
AACAUCCAGAAGGAAAUCGAUAGACUGAACGAGGUGGCAAAGAACCUGAAUGAGU
CCCUGAUUGACCUGCAAGAGCUCGGGAAGUACGAGCAGUAUAUCAAGUGGCCUUG
GUACAUCUGGCUGGGCUUCAUCGCGGGCCUGAUCGCCAUCGUUAUGGUGACGAUC
AUGCUGUGCUGCAUGACCAGUUGCUGUAGCUGCCUGAAGGGCUGCUGCAGCUGCG
GCAGCUGUUGCAAGUUCGACGAGGACGACAGCGAGCCUGUGCUGAAGGGCGUUAA
GCUGCACUACACCUGA
SEQ ID NO: 14, amino acid sequence of S protein comprising
the following mutations: delta variant mutations of T19R,
V70F, T95I, G142D, E156Δ, F157Δ, R158G, A222V, W258L, K417N,
L452R, T478K, D614G, P681R, and D950N; a beta variant mu-
tation of E484K; a variant mutation of N440K; S6P (F817P,
A892P, A899P, A942P, K986P, and V987P); and furin-like
cleavage site mutations (R682S and R685G):
MFVFLVLLPLVSSQCVNLRTRTQLPPAYTNSFTRGVYYPDKVFRSSVLHSTQDLFLPFFSN
VTWFHAIHFSGTNGTKRFDNPVLPFNDGVYFASIEKSNIIRGWIFGTTLDSKTQSLLIVNN
ATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSVLEPLVDLPIGINITRFQTLLAL
HRSYLTPGDSSSGLTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGNIADYNYK
LPDDFTGCVIAWNSNKLDSKVGGNYNYRYRLFRKSNLKPFERDISTEIYQAGSKPCNGV
KGFNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVITPGTNT
SNQVAVLYQGVNCTEVPVAIHADQLTPTWRVYSTGSNVFQTRAGCLIGAEHVNNSYEC
DIPIGAGICASYQTQTNSRSRAGSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQV
KQIYKTPPIKDFGGFNFSQILPDPSKPSKRSPIEDLLFNKVTLADAGFIKQYGDCLGDIAAR
DLICAQKFNGLTVLPPLLTDEMIAQYTSALLAGTITSGWTFGAGPALQIPFPMQMAYRFN
GIGVTQNVLYENQKLIANQFNSAIGKIQDSLSSTPSALGKLQNVVNQNAQALNTLVKQLS
SNFGAISSVLNDILSRLDPPEAEVQIDRLITGRLQSLQTYVTQQLIRAAEIRASANLAATKM
SECVLGQSKRVDFCGKGYHLMSFPQSAPHGVVFLHVTYVPAQEKNFTTAPAICHDGKA
HFPREGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDEDDSEPVLKGV
KLHYT.
SEQ ID NO: 15, optimized DNA sequence encoding
SEQ ID NO: 14:
ATGTTCGTGTTCCTGGTGCTGCTGCCTCTGGTCAGCAGCCAGTGCGTGAACCTGAGA
ACAAGAACACAGCTTCCTCCAGCCTACACAAACTCTTTTACACGGGGCGTGTACTAT
CCTGACAAGGTGTTCCGGTCCAGCGTGCTGCACTCAACCCAAGACCTGTTCCTGCCCT
TCTTCAGCAACGTCACCTGGTTCCACGCCATCCACTTCTCTGGCACCAATGGCACAAA
GCGATTCGATAACCCCGTGCTGCCTTTCAACGACGGCGTGTACTTTGCCTCCATCGAG
AAGTCCAACATCATCCGGGGCTGGATCTTCGGGACCACACTGGATAGCAAGACCCAG
TCTCTGCTGATCGTAAACAACGCCACCAACGTGGTCATCAAGGTGTGCGAGTTCCAG
TTCTGCAACGACCCTTTCCTCGATGTGTACTACCACAAGAACAACAAGTCTTGGATG
GAATCGGGCGTGTATAGCAGCGCCAACAACTGCACCTTCGAATACGTGAGCCAGCCT
TTCCTGATGGACCTGGAAGGCAAACAAGGCAATTTTAAGAACCTGAGAGAATTCGTG
TTCAAAAATATAGACGGCTATTTCAAGATCTACAGCAAGCACACCCCTATTAATCTG
GTGCGGGATCTGCCTCAGGGCTTCAGCGTCCTCGAACCTCTGGTGGACCTGCCAATC
GGCATCAACATTACAAGATTCCAGACGCTGCTCGCTCTGCACAGATCTTACCTGACC
CCTGGCGACAGCAGCAGCGGCCTGACCGCCGGCGCCGCCGCTTACTACGTGGGCTAC
CTGCAGCCTAGAACCTTTCTGCTGAAGTACAACGAGAACGGCACCATCACTGATGCC
GTGGATTGCGCCCTGGACCCTCTGTCCGAAACCAAATGTACACTGAAGTCTTTTACC
GTGGAAAAAGGAATCTACCAGACTTCCAACTTCCGGGTGCAGCCGACCGAGAGCAT
CGTGCGGTTCCCTAACATCACAAACCTGTGCCCCTTTGGCGAGGTGTTCAACGCCAC
AAGATTTGCTAGCGTGTACGCCTGGAATAGAAAGAGAATCAGCAACTGCGTGGCCG
ATTACAGCGTGCTGTACAATAGCGCCTCTTTCAGCACCTTCAAATGCTACGGCGTGA
GCCCCACCAAGCTGAACGATCTGTGTTTTACAAACGTGTATGCCGACTCATTCGTAAT
CAGGGGCGATGAGGTGAGACAGATCGCTCCTGGACAGACAGGCAAcATCGCGGACT
ACAACTATAAGCTGCCTGATGACTTCACAGGATGTGTGATCGCATGGAACTCCAATA
AGCTCGACAGCAAGGTGGGCGGAAATTACAATTACCGCTACAGACTGTTTAGAAAG
AGCAATCTGAAACCTTTCGAGAGAGACATCAGCACAGAGATCTACCAGGCCGGCAG
CAAGCCCTGTAACGGCGTCAAGGGCTTCAACTGCTACTTCCCCCTGCAGAGCTACGG
CTTCCAGCCTACCAACGGCGTGGGATACCAGCCTTACAGAGTGGTGGTGCTGAGCTT
CGAGCTGCTGCATGCTCCTGCTACAGTGTGTGGTCCTAAGAAGAGCACCAACCTGGT
TAAGAACAAGTGCGTGAATTTTAACTTCAATGGACTGACCGGAACCGGCGTGCTGAC
CGAAAGCAACAAGAAATTCCTGCCTTTTCAGCAGTTTGGCAGAGACATCGCCGACAC
CACCGACGCCGTGAGAGATCCACAAACCCTGGAAATCCTGGACATCACACCTTGCTC
ATTTGGAGGGGTGTCGGTGATCACACCTGGCACCAACACCAGCAACCAGGTGGCCGT
GCTGTACCAGGGAGTGAATTGTACCGAGGTCCCCGTGGCCATTCACGCCGACCAGCT
GACCCCTACCTGGCGGGTGTACTCCACCGGCTCTAACGTATTCCAGACCAGAGCCGG
CTGTCTGATCGGCGCAGAACACGTGAACAATAGCTACGAGTGCGACATCCCTATCGG
AGCCGGGATCTGCGCTAGCTACCAGACCCAGACAAACTCCAGAAGCAGAGCCGGAA
GCGTGGCCAGCCAGTCTATCATCGCCTACACCATGAGCCTGGGCGCCGAAAACAGCG
TTGCCTACAGCAACAATTCTATCGCCATCCCTACAAACTTCACCATCTCCGTGACCAC
CGAGATCCTGCCTGTCAGCATGACAAAGACCAGCGTAGACTGCACAATGTACATCTG
CGGAGATTCCACCGAGTGTAGTAACCTCCTGCTGCAATACGGATCTTTCTGTACTCAG
CTGAACAGAGCCCTGACCGGCATCGCCGTTGAACAGGACAAGAACACCCAGGAGGT
TTTCGCCCAGGTTAAGCAGATCTACAAAACCCCTCCTATCAAGGACTTCGGAGGCTT
TAACTTCTCCCAGATCCTGCCCGACCCCAGCAAGCCCAGCAAGCGGAGCCCCATCGA
GGACCTGCTGTTCAACAAGGTGACCCTGGCCGACGCCGGCTTCATCAAACAGTACGG
CGATTGCCTGGGAGACATCGCCGCTAGAGATCTAATTTGCGCCCAAAAGTTTAACGG
CCTGACAGTGCTGCCTCCACTGCTGACAGACGAGATGATCGCCCAGTACACATCTGC
CCTGCTGGCTGGTACCATCACATCTGGCTGGACCTTTGGCGCCGGCCCCGCCCTCCAG
ATCCCTTTCCCCATGCAGATGGCCTACCGGTTCAACGGCATCGGCGTGACCCAGAAC
GTGCTGTACGAAAACCAGAAACTGATCGCCAACCAGTTCAATAGCGCGATCGGCAA
AATCCAGGATAGCCTCAGCTCTACACCCAGCGCTCTTGGCAAGCTGCAAAACGTGGT
GAACCAGAATGCCCAGGCCCTTAACACCCTGGTGAAGCAGCTATCCTCTAATTTCGG
TGCCATCAGCAGCGTGCTGAATGATATCCTGAGCAGACTGGACCCCCCTGAGGCCGA
AGTGCAGATCGACAGACTGATCACCGGAAGACTGCAGAGCCTGCAAACCTACGTGA
CCCAGCAACTGATCCGGGCCGCAGAAATCCGGGCCTCCGCTAACCTGGCCGCTACCA
AGATGAGCGAGTGCGTGCTGGGTCAAAGCAAGCGCGTGGACTTCTGTGGAAAAGGC
TACCACCTGATGAGCTTCCCTCAGAGCGCTCCACACGGCGTGGTGTTCCTGCATGTG
ACTTACGTGCCTGCCCAGGAAAAGAACTTCACCACCGCCCCTGCCATTTGTCACGAC
GGCAAGGCCCACTTCCCCCGGGAAGGCGTGTTTGTGTCTAACGGAACACACTGGTTT
GTGACTCAAAGAAACTTCTACGAGCCACAGATCATCACCACAGATAACACCTTCGTC
AGCGGCAACTGCGACGTGGTGATCGGCATCGTGAACAATACTGTGTACGACCCCCTG
CAGCCAGAGCTCGATTCTTTCAAAGAGGAACTGGATAAGTACTTCAAGAACCACACA
TCCCCCGACGTCGACCTGGGCGATATCAGCGGCATTAACGCCAGCGTGGTGAACATC
CAGAAGGAAATCGATAGACTGAACGAGGTGGCAAAGAACCTGAATGAGTCCCTGAT
TGACCTGCAAGAGCTCGGGAAGTACGAGCAGTATATCAAGTGGCCTTGGTACATCTG
GCTGGGCTTCATCGCGGGCCTGATCGCCATCGTTATGGTGACGATCATGCTGTGCTGC
ATGACCAGTTGCTGTAGCTGCCTGAAGGGCTGCTGCAGCTGCGGCAGCTGTTGCAAG
TTCGACGAGGACGACAGCGAGCCTGTGCTGAAGGGCGTTAAGCTGCACTACACCTGA
SEQ ID NO: 16, optimized RNA sequence encoding
SEQ ID NO: 14:
AUGUUCGUGUUCCUGGUGCUGCUGCCUCUGGUCAGCAGCCAGUGCGUGAACCUGA
GAACAAGAACACAGCUUCCUCCAGCCUACACAAACUCUUUUACACGGGGCGUGUA
CUAUCCUGACAAGGUGUUCCGGUCCAGCGUGCUGCACUCAACCCAAGACCUGUUC
CUGCCCUUCUUCAGCAACGUCACCUGGUUCCACGCCAUCCACUUCUCUGGCACCAA
UGGCACAAAGCGAUUCGAUAACCCCGUGCUGCCUUUCAACGACGGCGUGUACUUU
GCCUCCAUCGAGAAGUCCAACAUCAUCCGGGGCUGGAUCUUCGGGACCACACUGG
AUAGCAAGACCCAGUCUCUGCUGAUCGUAAACAACGCCACCAACGUGGUCAUCAA
GGUGUGCGAGUUCCAGUUCUGCAACGACCCUUUCCUCGAUGUGUACUACCACAAG
AACAACAAGUCUUGGAUGGAAUCGGGCGUGUAUAGCAGCGCCAACAACUGCACCU
UCGAAUACGUGAGCCAGCCUUUCCUGAUGGACCUGGAAGGCAAACAAGGCAAUUU
UAAGAACCUGAGAGAAUUCGUGUUCAAAAAUAUAGACGGCUAUUUCAAGAUCUAC
AGCAAGCACACCCCUAUUAAUCUGGUGCGGGAUCUGCCUCAGGGCUUCAGCGUCC
UCGAACCUCUGGUGGACCUGCCAAUCGGCAUCAACAUUACAAGAUUCCAGACGCU
GCUCGCUCUGCACAGAUCUUACCUGACCCCUGGCGACAGCAGCAGCGGCCUGACCG
CCGGCGCCGCCGCUUACUACGUGGGCUACCUGCAGCCUAGAACCUUUCUGCUGAA
GUACAACGAGAACGGCACCAUCACUGAUGCCGUGGAUUGCGCCCUGGACCCUCUG
UCCGAAACCAAAUGUACACUGAAGUCUUUUACCGUGGAAAAAGGAAUCUACCAGA
CUUCCAACUUCCGGGUGCAGCCGACCGAGAGCAUCGUGCGGUUCCCUAACAUCAC
AAACCUGUGCCCCUUUGGCGAGGUGUUCAACGCCACAAGAUUUGCUAGCGUGUAC
GCCUGGAAUAGAAAGAGAAUCAGCAACUGCGUGGCCGAUUACAGCGUGCUGUACA
AUAGCGCCUCUUUCAGCACCUUCAAAUGCUACGGCGUGAGCCCCACCAAGCUGAA
CGAUCUGUGUUUUACAAACGUGUAUGCCGACUCAUUCGUAAUCAGGGGCGAUGAG
GUGAGACAGAUCGCUCCUGGACAGACAGGCAACAUCGCGGACUACAACUAUAAGC
UGCCUGAUGACUUCACAGGAUGUGUGAUCGCAUGGAACUCCAAUAAGCUCGACAG
CAAGGUGGGCGGAAAUUACAAUUACCGCUACAGACUGUUUAGAAAGAGCAAUCUG
AAACCUUUCGAGAGAGACAUCAGCACAGAGAUCUACCAGGCCGGCAGCAAGCCCU
GUAACGGCGUCAAGGGCUUCAACUGCUACUUCCCCCUGCAGAGCUACGGCUUCCA
GCCUACCAACGGCGUGGGAUACCAGCCUUACAGAGUGGUGGUGCUGAGCUUCGAG
CUGCUGCAUGCUCCUGCUACAGUGUGUGGUCCUAAGAAGAGCACCAACCUGGUUA
AGAACAAGUGCGUGAAUUUUAACUUCAAUGGACUGACCGGAACCGGCGUGCUGAC
CGAAAGCAACAAGAAAUUCCUGCCUUUUCAGCAGUUUGGCAGAGACAUCGCCGAC
ACCACCGACGCCGUGAGAGAUCCACAAACCCUGGAAAUCCUGGACAUCACACCUU
GCUCAUUUGGAGGGGUGUCGGUGAUCACACCUGGCACCAACACCAGCAACCAGGU
GGCCGUGCUGUACCAGGGAGUGAAUUGUACCGAGGUCCCCGUGGCCAUUCACGCC
GACCAGCUGACCCCUACCUGGCGGGUGUACUCCACCGGCUCUAACGUAUUCCAGA
CCAGAGCCGGCUGUCUGAUCGGCGCAGAACACGUGAACAAUAGCUACGAGUGCGA
CAUCCCUAUCGGAGCCGGGAUCUGCGCUAGCUACCAGACCCAGACAAACUCCAGA
AGCAGAGCCGGAAGCGUGGCCAGCCAGUCUAUCAUCGCCUACACCAUGAGCCUGG
GCGCCGAAAACAGCGUUGCCUACAGCAACAAUUCUAUCGCCAUCCCUACAAACUU
CACCAUCUCCGUGACCACCGAGAUCCUGCCUGUCAGCAUGACAAAGACCAGCGUA
GACUGCACAAUGUACAUCUGCGGAGAUUCCACCGAGUGUAGUAACCUCCUGCUGC
AAUACGGAUCUUUCUGUACUCAGCUGAACAGAGCCCUGACCGGCAUCGCCGUUGA
ACAGGACAAGAACACCCAGGAGGUUUUCGCCCAGGUUAAGCAGAUCUACAAAACC
CCUCCUAUCAAGGACUUCGGAGGCUUUAACUUCUCCCAGAUCCUGCCCGACCCCAG
CAAGCCCAGCAAGCGGAGCCCCAUCGAGGACCUGCUGUUCAACAAGGUGACCCUG
GCCGACGCCGGCUUCAUCAAACAGUACGGCGAUUGCCUGGGAGACAUCGCCGCUA
GAGAUCUAAUUUGCGCCCAAAAGUUUAACGGCCUGACAGUGCUGCCUCCACUGCU
GACAGACGAGAUGAUCGCCCAGUACACAUCUGCCCUGCUGGCUGGUACCAUCACA
UCUGGCUGGACCUUUGGCGCCGGCCCCGCCCUCCAGAUCCCUUUCCCCAUGCAGAU
GGCCUACCGGUUCAACGGCAUCGGCGUGACCCAGAACGUGCUGUACGAAAACCAG
AAACUGAUCGCCAACCAGUUCAAUAGCGCGAUCGGCAAAAUCCAGGAUAGCCUCA
GCUCUACACCCAGCGCUCUUGGCAAGCUGCAAAACGUGGUGAACCAGAAUGCCCA
GGCCCUUAACACCCUGGUGAAGCAGCUAUCCUCUAAUUUCGGUGCCAUCAGCAGC
GUGCUGAAUGAUAUCCUGAGCAGACUGGACCCCCCUGAGGCCGAAGUGCAGAUCG
ACAGACUGAUCACCGGAAGACUGCAGAGCCUGCAAACCUACGUGACCCAGCAACU
GAUCCGGGCCGCAGAAAUCCGGGCCUCCGCUAACCUGGCCGCUACCAAGAUGAGC
GAGUGCGUGCUGGGUCAAAGCAAGCGCGUGGACUUCUGUGGAAAAGGCUACCACC
UGAUGAGCUUCCCUCAGAGCGCUCCACACGGCGUGGUGUUCCUGCAUGUGACUUA
CGUGCCUGCCCAGGAAAAGAACUUCACCACCGCCCCUGCCAUUUGUCACGACGGCA
AGGCCCACUUCCCCCGGGAAGGCGUGUUUGUGUCUAACGGAACACACUGGUUUGU
GACUCAAAGAAACUUCUACGAGCCACAGAUCAUCACCACAGAUAACACCUUCGUC
AGCGGCAACUGCGACGUGGUGAUCGGCAUCGUGAACAAUACUGUGUACGACCCCC
UGCAGCCAGAGCUCGAUUCUUUCAAAGAGGAACUGGAUAAGUACUUCAAGAACCA
CACAUCCCCCGACGUCGACCUGGGCGAUAUCAGCGGCAUUAACGCCAGCGUGGUG
AACAUCCAGAAGGAAAUCGAUAGACUGAACGAGGUGGCAAAGAACCUGAAUGAGU
CCCUGAUUGACCUGCAAGAGCUCGGGAAGUACGAGCAGUAUAUCAAGUGGCCUUG
GUACAUCUGGCUGGGCUUCAUCGCGGGCCUGAUCGCCAUCGUUAUGGUGACGAUC
AUGCUGUGCUGCAUGACCAGUUGCUGUAGCUGCCUGAAGGGCUGCUGCAGCUGCG
GCAGCUGUUGCAAGUUCGACGAGGACGACAGCGAGCCUGUGCUGAAGGGCGUUAA
GCUGCACUACACCUGA
SEQ ID NO: 17, DNA sequence of 3′ UTR:
GCTCGCTTTCTTGCTGTCCAATTTCTATTAAAGGTTCCTTTGTTCCCTAAGTCCAACTA
CTAAACTGGGGGATATTATGAAGGGCCTTGAGCATCTGGATTCTGCCTAATAAAAAA
CATTTATTTTCATTGC
SEQ ID NO: 18, RNA sequence of 3′ UTR:
GCUCGCUUUCUUGCUGUCCAAUUUCUAUUAAAGGUUCCUUUGUUCCCUAAGUCCA
ACUACUAAACUGGGGGAUAUUAUGAAGGGCCUUGAGCAUCUGGAUUCUGCCUAAU
AAAAAACAUUUAUUUUCAUUGC
SEQ ID NO: 19, DNA sequence of β-globulin 5′UTR:
ACATTTGCTTCTGACACAACTGTGTTCACTAGCAACCTCAAACAGACACC
SEQ ID NO: 20, RNA sequence of β-globulin 5′UTR:
ACAUUUGCUUCUGACACAACUGUGUUCACUAGCAACCUCAAACAGACACC
SEQ ID NO: 21, DNA sequence of SYS UTR 2.0:
GGCGCTCGAGCAGGTTCAGAAGGAGATCAAAAACCCCCAAGGATCAAACGCCACC
SEQ ID NO: 22, RNA sequence of SYS UTR 2.0:
GGCGCUCGAGCAGGUUCAGAAGGAGAUCAAAAACCCCCAAGGAUCAAACGCCACC
SEQ ID NO: 23, DNA sequence of SYS UTR 1.0:
GGGCGCTCGAGCAGGTTCAGAAGGAGATCAAAAACCCCCAAGGATCAAAC
SEQ ID NO: 24, RNA sequence of SYS UTR 1.0:
GGGCGCUCGAGCAGGUUCAGAAGGAGAUCAAAAACCCCCAAGGAUCAAAC
SEQ ID NO: 25, DNA sequence of SYS4 5′UTR:
GGCGCACGAGCAGGGAGAGAAGGAGATCAAAAACCCCCAAGGATCAAACGCCACC
SEQ ID NO: 26, RNA sequence of SYS4 5′UTR:
GGCGCACGAGCAGGGAGAGAAGGAGAUCAAAAACCCCCAAGGAUCAAACGCCACC
SEQ ID NO: 56 (DNA) and SEQ ID NO: 27
(RNA) sequence of polyA 40:
AAAAAAAAAAAAAAAAAAAAAAAAAAAAAAAAAAAAAAAA
SEQ ID NO: 57 (DNA) and SEQ ID NO: 28
(RNA) sequence of polyA 60:
AAAAAAAAAAAAAAAAAAAAAAAAAAAAAAAAAAAAAAAAAAAAAAAAAAAAAA
AAAAAAGAAAAAAAAAAAAAAAAAAAAAAAAAAAAAAAAAAAAAAAAAAAAAAA
AAAAAAAAAAAAA
SEQ ID NO: 29, DNA sequence of polyA signal HSV:
CGGCAATAAAAAGACAGAATAAAACGCACGGTGTTGGGTCGTTTGTTC
SEQ ID NO: 30, RNA sequence of polyA signal HSV:
CGGCAAUAAAAAGACAGAAUAAAACGCACGGUGUUGGGUCGUUUGUUC
SEQ ID NO: 31, DNA sequence of an exemplified
5′UTR-deltaC S6P-3′ UTR-polyA:
GGACATTTGCTTCTGACACAACTGTGTTCACTAGCAACCTCAAACAGACACCGCCAC
CATGTTCGTGTTCCTGGTGCTGCTGCCTCTGGTCAGCAGCCAGTGCGTGAACCTGAGA
ACAAGAACACAGCTTCCTCCAGCCTACACAAACTCTTTTACACGGGGCGTGTACTAT
CCTGACAAGGTGTTCCGGTCCAGCGTGCTGCACTCAACCCAAGACCTGTTCCTGCCCT
TCTTCAGCAACGTCACCTGGTTCCACGCCATCCACGTGTCTGGCACCAATGGCACAA
AGCGATTCGATAACCCCGTGCTGCCTTTCAACGACGGCGTGTACTTTGCCTCCATCGA
GAAGTCCAACATCATCCGGGGCTGGATCTTCGGGACCACACTGGATAGCAAGACCCA
GTCTCTGCTGATCGTAAACAACGCCACCAACGTGGTCATCAAGGTGTGCGAGTTCCA
GTTCTGCAACGACCCTTTCCTCGATGTGTACTACCACAAGAACAACAAGTCTTGGAT
GGAATCGGGCGTGTATAGCAGCGCCAACAACTGCACCTTCGAATACGTGAGCCAGCC
TTTCCTGATGGACCTGGAAGGCAAACAAGGCAATTTTAAGAACCTGAGAGAATTCGT
GTTCAAAAATATAGACGGCTATTTCAAGATCTACAGCAAGCACACCCCTATTAATCT
GGTGCGGGATCTGCCTCAGGGCTTCAGCGCCCTCGAACCTCTGGTGGACCTGCCAAT
CGGCATCAACATTACAAGATTCCAGACGCTGCTCGCTCTGCACAGATCTTACCTGAC
CCCTGGCGACAGCAGCAGCGGCTGGACCGCCGGCGCCGCCGCTTACTACGTGGGCTA
CCTGCAGCCTAGAACCTTTCTGCTGAAGTACAACGAGAACGGCACCATCACTGATGC
CGTGGATTGCGCCCTGGACCCTCTGTCCGAAACCAAATGTACACTGAAGTCTTTTACC
GTGGAAAAAGGAATCTACCAGACTTCCAACTTCCGGGTGCAGCCGACCGAGAGCAT
CGTGCGGTTCCCTAACATCACAAACCTGTGCCCCTTTGGCGAGGTGTTCAACGCCAC
AAGATTTGCTAGCGTGTACGCCTGGAATAGAAAGAGAATCAGCAACTGCGTGGCCG
ATTACAGCGTGCTGTACAATAGCGCCTCTTTCAGCACCTTCAAATGCTACGGCGTGA
GCCCCACCAAGCTGAACGATCTGTGTTTTACAAACGTGTATGCCGACTCATTCGTAAT
CAGGGGCGATGAGGTGAGACAGATCGCTCCTGGACAGACAGGCAAAATCGCGGACT
ACAACTATAAGCTGCCTGATGACTTCACAGGATGTGTGATCGCATGGAACTCCAATA
ACCTCGACAGCAAGGTGGGCGGAAATTACAATTACCGCTACAGACTGTTTAGAAAG
AGCAATCTGAAACCTTTCGAGAGAGACATCAGCACAGAGATCTACCAGGCCGGCAG
CAAGCCCTGTAACGGCGTCGAGGGCTTCAACTGCTACTTCCCCCTGCAGAGCTACGG
CTTCCAGCCTACCAACGGCGTGGGATACCAGCCTTACAGAGTGGTGGTGCTGAGCTT
CGAGCTGCTGCATGCTCCTGCTACAGTGTGTGGTCCTAAGAAGAGCACCAACCTGGT
TAAGAACAAGTGCGTGAATTTTAACTTCAATGGACTGACCGGAACCGGCGTGCTGAC
CGAAAGCAACAAGAAATTCCTGCCTTTTCAGCAGTTTGGCAGAGACATCGCCGACAC
CACCGACGCCGTGAGAGATCCACAAACCCTGGAAATCCTGGACATCACACCTTGCTC
ATTTGGAGGGGTGTCGGTGATCACACCTGGCACCAACACCAGCAACCAGGTGGCCGT
GCTGTACCAGGGAGTGAATTGTACCGAGGTCCCCGTGGCCATTCACGCCGACCAGCT
GACCCCTACCTGGCGGGTGTACTCCACCGGCTCTAACGTATTCCAGACCAGAGCCGG
CTGTCTGATCGGCGCAGAACACGTGAACAATAGCTACGAGTGCGACATCCCTATCGG
AGCCGGGATCTGCGCTAGCTACCAGACCCAGACAAACTCCAGAAGCAGAGCCGGAA
GCGTGGCCAGCCAGTCTATCATCGCCTACACCATGAGCCTGGGCGCCGAAAACAGCG
TTGCCTACAGCAACAATTCTATCGCCATCCCTACAAACTTCACCATCTCCGTGACCAC
CGAGATCCTGCCTGTCAGCATGACAAAGACCAGCGTAGACTGCACAATGTACATCTG
CGGAGATTCCACCGAGTGTAGTAACCTCCTGCTGCAATACGGATCTTTCTGTACTCAG
CTGAACAGAGCCCTGACCGGCATCGCCGTTGAACAGGACAAGAACACCCAGGAGGT
TTTCGCCCAGGTTAAGCAGATCTACAAAACCCCTCCTATCAAGGACTTCGGAGGCTT
TAACTTCTCCCAGATCCTGCCCGACCCCAGCAAGCCCAGCAAGCGGAGCCCCATCGA
GGACCTGCTGTTCAACAAGGTGACCCTGGCCGACGCCGGCTTCATCAAACAGTACGG
CGATTGCCTGGGAGACATCGCCGCTAGAGATCTAATTTGCGCCCAAAAGTTTAACGG
CCTGACAGTGCTGCCTCCACTGCTGACAGACGAGATGATCGCCCAGTACACATCTGC
CCTGCTGGCTGGTACCATCACATCTGGCTGGACCTTTGGCGCCGGCCCCGCCCTCCAG
ATCCCTTTCCCCATGCAGATGGCCTACCGGTTCAACGGCATCGGCGTGACCCAGAAC
GTGCTGTACGAAAACCAGAAACTGATCGCCAACCAGTTCAATAGCGCGATCGGCAA
AATCCAGGATAGCCTCAGCTCTACACCCAGCGCTCTTGGCAAGCTGCAAAACGTGGT
GAACCAGAATGCCCAGGCCCTTAACACCCTGGTGAAGCAGCTATCCTCTAATTTCGG
TGCCATCAGCAGCGTGCTGAATGATATCCTGAGCAGACTGGACCCCCCTGAGGCCGA
AGTGCAGATCGACAGACTGATCACCGGAAGACTGCAGAGCCTGCAAACCTACGTGA
CCCAGCAACTGATCCGGGCCGCAGAAATCCGGGCCTCCGCTAACCTGGCCGCTACCA
AGATGAGCGAGTGCGTGCTGGGTCAAAGCAAGCGCGTGGACTTCTGTGGAAAAGGC
TACCACCTGATGAGCTTCCCTCAGAGCGCTCCACACGGCGTGGTGTTCCTGCATGTG
ACTTACGTGCCTGCCCAGGAAAAGAACTTCACCACCGCCCCTGCCATTTGTCACGAC
GGCAAGGCCCACTTCCCCCGGGAAGGCGTGTTTGTGTCTAACGGAACACACTGGTTT
GTGACTCAAAGAAACTTCTACGAGCCACAGATCATCACCACAGATAACACCTTCGTC
AGCGGCAACTGCGACGTGGTGATCGGCATCGTGAACAATACTGTGTACGACCCCCTG
CAGCCAGAGCTCGATTCTTTCAAAGAGGAACTGGATAAGTACTTCAAGAACCACACA
TCCCCCGACGTCGACCTGGGCGATATCAGCGGCATTAACGCCAGCGTGGTGAACATC
CAGAAGGAAATCGATAGACTGAACGAGGTGGCAAAGAACCTGAATGAGTCCCTGAT
TGACCTGCAAGAGCTCGGGAAGTACGAGCAGTATATCAAGTGGCCTTGGTACATCTG
GCTGGGCTTCATCGCGGGCCTGATCGCCATCGTTATGGTGACGATCATGCTGTGCTGC
ATGACCAGTTGCTGTAGCTGCCTGAAGGGCTGCTGCAGCTGCGGCAGCTGTTGCAAG
TTCGACGAGGACGACAGCGAGCCTGTGCTGAAGGGCGTTAAGCTGCACTACACCTGA
GCTCGCTTTCTTGCTGTCCAATTTCTATTAAAGGTTCCTTTGTTCCCTAAGTCCAACTA
CTAAACTGGGGGATATTATGAAGGGCCTTGAGCATCTGGATTCTGCCTAATAAAAAA
CATTTATTTTCATTGCCAATAGGCCGAAATCGGCAAGCGCGATCGCAAAAAAAAAAA
AAAAAAAAAAAAAAAAAAAAAAAAAAAAAAAAAAAAAAAGAAAAAAAAAAAAAA
AAAAAAAAAAAAAAAAAAAAAAAAAAAAAAAAAAAAAAAAAAAAA
SEQ ID NO: 32, RNA sequence of an exemplified
5′UTR-deltaC S6P-3′ UTR-polyA:
GGACAUUUGCUUCUGACACAACUGUGUUCACUAGCAACCUCAAACAGACACCGCC
ACCAUGUUCGUGUUCCUGGUGCUGCUGCCUCUGGUCAGCAGCCAGUGCGUGAACC
UGAGAACAAGAACACAGCUUCCUCCAGCCUACACAAACUCUUUUACACGGGGCGU
GUACUAUCCUGACAAGGUGUUCCGGUCCAGCGUGCUGCACUCAACCCAAGACCUG
UUCCUGCCCUUCUUCAGCAACGUCACCUGGUUCCACGCCAUCCACGUGUCUGGCAC
CAAUGGCACAAAGCGAUUCGAUAACCCCGUGCUGCCUUUCAACGACGGCGUGUAC
UUUGCCUCCAUCGAGAAGUCCAACAUCAUCCGGGGCUGGAUCUUCGGGACCACAC
UGGAUAGCAAGACCCAGUCUCUGCUGAUCGUAAACAACGCCACCAACGUGGUCAU
CAAGGUGUGCGAGUUCCAGUUCUGCAACGACCCUUUCCUCGAUGUGUACUACCAC
AAGAACAACAAGUCUUGGAUGGAAUCGGGCGUGUAUAGCAGCGCCAACAACUGCA
CCUUCGAAUACGUGAGCCAGCCUUUCCUGAUGGACCUGGAAGGCAAACAAGGCAA
UUUUAAGAACCUGAGAGAAUUCGUGUUCAAAAAUAUAGACGGCUAUUUCAAGAU
CUACAGCAAGCACACCCCUAUUAAUCUGGUGCGGGAUCUGCCUCAGGGCUUCAGC
GCCCUCGAACCUCUGGUGGACCUGCCAAUCGGCAUCAACAUUACAAGAUUCCAGA
CGCUGCUCGCUCUGCACAGAUCUUACCUGACCCCUGGCGACAGCAGCAGCGGCUG
GACCGCCGGCGCCGCCGCUUACUACGUGGGCUACCUGCAGCCUAGAACCUUUCUGC
UGAAGUACAACGAGAACGGCACCAUCACUGAUGCCGUGGAUUGCGCCCUGGACCC
UCUGUCCGAAACCAAAUGUACACUGAAGUCUUUUACCGUGGAAAAAGGAAUCUAC
CAGACUUCCAACUUCCGGGUGCAGCCGACCGAGAGCAUCGUGCGGUUCCCUAACA
UCACAAACCUGUGCCCCUUUGGCGAGGUGUUCAACGCCACAAGAUUUGCUAGCGU
GUACGCCUGGAAUAGAAAGAGAAUCAGCAACUGCGUGGCCGAUUACAGCGUGCUG
UACAAUAGCGCCUCUUUCAGCACCUUCAAAUGCUACGGCGUGAGCCCCACCAAGC
UGAACGAUCUGUGUUUUACAAACGUGUAUGCCGACUCAUUCGUAAUCAGGGGCGA
UGAGGUGAGACAGAUCGCUCCUGGACAGACAGGCAAAAUCGCGGACUACAACUAU
AAGCUGCCUGAUGACUUCACAGGAUGUGUGAUCGCAUGGAACUCCAAUAACCUCG
ACAGCAAGGUGGGCGGAAAUUACAAUUACCGCUACAGACUGUUUAGAAAGAGCAA
UCUGAAACCUUUCGAGAGAGACAUCAGCACAGAGAUCUACCAGGCCGGCAGCAAG
CCCUGUAACGGCGUCGAGGGCUUCAACUGCUACUUCCCCCUGCAGAGCUACGGCU
UCCAGCCUACCAACGGCGUGGGAUACCAGCCUUACAGAGUGGUGGUGCUGAGCUU
CGAGCUGCUGCAUGCUCCUGCUACAGUGUGUGGUCCUAAGAAGAGCACCAACCUG
GUUAAGAACAAGUGCGUGAAUUUUAACUUCAAUGGACUGACCGGAACCGGCGUGC
UGACCGAAAGCAACAAGAAAUUCCUGCCUUUUCAGCAGUUUGGCAGAGACAUCGC
CGACACCACCGACGCCGUGAGAGAUCCACAAACCCUGGAAAUCCUGGACAUCACA
CCUUGCUCAUUUGGAGGGGUGUCGGUGAUCACACCUGGCACCAACACCAGCAACC
AGGUGGCCGUGCUGUACCAGGGAGUGAAUUGUACCGAGGUCCCCGUGGCCAUUCA
CGCCGACCAGCUGACCCCUACCUGGCGGGUGUACUCCACCGGCUCUAACGUAUUCC
AGACCAGAGCCGGCUGUCUGAUCGGCGCAGAACACGUGAACAAUAGCUACGAGUG
CGACAUCCCUAUCGGAGCCGGGAUCUGCGCUAGCUACCAGACCCAGACAAACUCC
AGAAGCAGAGCCGGAAGCGUGGCCAGCCAGUCUAUCAUCGCCUACACCAUGAGCC
UGGGCGCCGAAAACAGCGUUGCCUACAGCAACAAUUCUAUCGCCAUCCCUACAAA
CUUCACCAUCUCCGUGACCACCGAGAUCCUGCCUGUCAGCAUGACAAAGACCAGC
GUAGACUGCACAAUGUACAUCUGCGGAGAUUCCACCGAGUGUAGUAACCUCCUGC
UGCAAUACGGAUCUUUCUGUACUCAGCUGAACAGAGCCCUGACCGGCAUCGCCGU
UGAACAGGACAAGAACACCCAGGAGGUUUUCGCCCAGGUUAAGCAGAUCUACAAA
ACCCCUCCUAUCAAGGACUUCGGAGGCUUUAACUUCUCCCAGAUCCUGCCCGACCC
CAGCAAGCCCAGCAAGCGGAGCCCCAUCGAGGACCUGCUGUUCAACAAGGUGACC
CUGGCCGACGCCGGCUUCAUCAAACAGUACGGCGAUUGCCUGGGAGACAUCGCCG
CUAGAGAUCUAAUUUGCGCCCAAAAGUUUAACGGCCUGACAGUGCUGCCUCCACU
GCUGACAGACGAGAUGAUCGCCCAGUACACAUCUGCCCUGCUGGCUGGUACCAUC
ACAUCUGGCUGGACCUUUGGCGCCGGCCCCGCCCUCCAGAUCCCUUUCCCCAUGCA
GAUGGCCUACCGGUUCAACGGCAUCGGCGUGACCCAGAACGUGCUGUACGAAAAC
CAGAAACUGAUCGCCAACCAGUUCAAUAGCGCGAUCGGCAAAAUCCAGGAUAGCC
UCAGCUCUACACCCAGCGCUCUUGGCAAGCUGCAAAACGUGGUGAACCAGAAUGC
CCAGGCCCUUAACACCCUGGUGAAGCAGCUAUCCUCUAAUUUCGGUGCCAUCAGC
AGCGUGCUGAAUGAUAUCCUGAGCAGACUGGACCCCCCUGAGGCCGAAGUGCAGA
UCGACAGACUGAUCACCGGAAGACUGCAGAGCCUGCAAACCUACGUGACCCAGCA
ACUGAUCCGGGCCGCAGAAAUCCGGGCCUCCGCUAACCUGGCCGCUACCAAGAUG
AGCGAGUGCGUGCUGGGUCAAAGCAAGCGCGUGGACUUCUGUGGAAAAGGCUACC
ACCUGAUGAGCUUCCCUCAGAGCGCUCCACACGGCGUGGUGUUCCUGCAUGUGAC
UUACGUGCCUGCCCAGGAAAAGAACUUCACCACCGCCCCUGCCAUUUGUCACGAC
GGCAAGGCCCACUUCCCCCGGGAAGGCGUGUUUGUGUCUAACGGAACACACUGGU
UUGUGACUCAAAGAAACUUCUACGAGCCACAGAUCAUCACCACAGAUAACACCUU
CGUCAGCGGCAACUGCGACGUGGUGAUCGGCAUCGUGAACAAUACUGUGUACGAC
CCCCUGCAGCCAGAGCUCGAUUCUUUCAAAGAGGAACUGGAUAAGUACUUCAAGA
ACCACACAUCCCCCGACGUCGACCUGGGCGAUAUCAGCGGCAUUAACGCCAGCGU
GGUGAACAUCCAGAAGGAAAUCGAUAGACUGAACGAGGUGGCAAAGAACCUGAAU
GAGUCCCUGAUUGACCUGCAAGAGCUCGGGAAGUACGAGCAGUAUAUCAAGUGGC
CUUGGUACAUCUGGCUGGGCUUCAUCGCGGGCCUGAUCGCCAUCGUUAUGGUGAC
GAUCAUGCUGUGCUGCAUGACCAGUUGCUGUAGCUGCCUGAAGGGCUGCUGCAGC
UGCGGCAGCUGUUGCAAGUUCGACGAGGACGACAGCGAGCCUGUGCUGAAGGGCG
UUAAGCUGCACUACACCUGAGCUCGCUUUCUUGCUGUCCAAUUUCUAUUAAAGGU
UCCUUUGUUCCCUAAGUCCAACUACUAAACUGGGGGAUAUUAUGAAGGGCCUUGA
GCAUCUGGAUUCUGCCUAAUAAAAAACAUUUAUUUUCAUUGCCAAUAGGCCGAAA
UCGGCAAGCGCGAUCGCAAAAAAAAAAAAAAAAAAAAAAAAAAAAAAAAAAAAA
AAAAAAAAAAAAAGAAAAAAAAAAAAAAAAAAAAAAAAAAAAAAAAAAAAAAAA
AAAAAAAAAAAAAAAAAAA
SEQ ID NO: 33, plasmid DNA sequence:
TCGCGCGTTTCGGTGATGACGGTGAAAACCTCTGACACATGCAGCTCCCGGAGACGG
TCACAGCTTGTCTGTAAGCGGATGCCGGGAGCAGACAAGCCCGTCAGGGCGCGTCA
GCGGGTGTTGGCGGGTGTCGGGGCTGGCTTAACTATGCGGCATCAGAGCAGATTGTA
CTGAGAGTGCACCATATGCGGTGTGAAATACCGCACAGATGCGTAAGGAGAAAATA
CCGCATCAGGCGCCATTCGCCATTCAGGCTGCGCAACTGTTGGGAAGGGCGATCGGT
GCGGGCCTCTTCGCTATTACGCCAGCTGGCGAAAGGGGGATGTGCTGCAAGGCGATT
AAGTTGGGTAACGCCAGGGTTTTCCCAGTCACGACGTTGTAAAACGACGGCCAGTGA
ATTCGAGCTCGGTACCTCGCGAATGCATCTAGATATCGGATCCCGGGCCCGTCGACT
GCAGAGGCCTGCATGCAAGCTTTAATACGACTCACTATAAGGACATTTGCTTCTGAC
ACAACTGTGTTCACTAGCAACCTCAAACAGACACCGCCACCATGTTCGTGTTCCTGG
TGCTGCTGCCTCTGGTCAGCAGCCAGTGCGTGAACCTGAGAACAAGAACACAGCTTC
CTCCAGCCTACACAAACTCTTTTACACGGGGCGTGTACTATCCTGACAAGGTGTTCCG
GTCCAGCGTGCTGCACTCAACCCAAGACCTGTTCCTGCCCTTCTTCAGCAACGTCACC
TGGTTCCACGCCATCCACGTGTCTGGCACCAATGGCACAAAGCGATTCGATAACCCC
GTGCTGCCTTTCAACGACGGCGTGTACTTTGCCTCCATCGAGAAGTCCAACATCATCC
GGGGCTGGATCTTCGGGACCACACTGGATAGCAAGACCCAGTCTCTGCTGATCGTAA
ACAACGCCACCAACGTGGTCATCAAGGTGTGCGAGTTCCAGTTCTGCAACGACCCTT
TCCTCGATGTGTACTACCACAAGAACAACAAGTCTTGGATGGAATCGGGCGTGTATA
GCAGCGCCAACAACTGCACCTTCGAATACGTGAGCCAGCCTTTCCTGATGGACCTGG
AAGGCAAACAAGGCAATTTTAAGAACCTGAGAGAATTCGTGTTCAAAAATATAGAC
GGCTATTTCAAGATCTACAGCAAGCACACCCCTATTAATCTGGTGCGGGATCTGCCT
CAGGGCTTCAGCGCCCTCGAACCTCTGGTGGACCTGCCAATCGGCATCAACATTACA
AGATTCCAGACGCTGCTCGCTCTGCACAGATCTTACCTGACCCCTGGCGACAGCAGC
AGCGGCTGGACCGCCGGCGCCGCCGCTTACTACGTGGGCTACCTGCAGCCTAGAACC
TTTCTGCTGAAGTACAACGAGAACGGCACCATCACTGATGCCGTGGATTGCGCCCTG
GACCCTCTGTCCGAAACCAAATGTACACTGAAGTCTTTTACCGTGGAAAAAGGAATC
TACCAGACTTCCAACTTCCGGGTGCAGCCGACCGAGAGCATCGTGCGGTTCCCTAAC
ATCACAAACCTGTGCCCCTTTGGCGAGGTGTTCAACGCCACAAGATTTGCTAGCGTG
TACGCCTGGAATAGAAAGAGAATCAGCAACTGCGTGGCCGATTACAGCGTGCTGTAC
AATAGCGCCTCTTTCAGCACCTTCAAATGCTACGGCGTGAGCCCCACCAAGCTGAAC
GATCTGTGTTTTACAAACGTGTATGCCGACTCATTCGTAATCAGGGGCGATGAGGTG
AGACAGATCGCTCCTGGACAGACAGGCAAAATCGCGGACTACAACTATAAGCTGCC
TGATGACTTCACAGGATGTGTGATCGCATGGAACTCCAATAACCTCGACAGCAAGGT
GGGCGGAAATTACAATTACCGCTACAGACTGTTTAGAAAGAGCAATCTGAAACCTTT
CGAGAGAGACATCAGCACAGAGATCTACCAGGCCGGCAGCAAGCCCTGTAACGGCG
TCGAGGGCTTCAACTGCTACTTCCCCCTGCAGAGCTACGGCTTCCAGCCTACCAACG
GCGTGGGATACCAGCCTTACAGAGTGGTGGTGCTGAGCTTCGAGCTGCTGCATGCTC
CTGCTACAGTGTGTGGTCCTAAGAAGAGCACCAACCTGGTTAAGAACAAGTGCGTGA
ATTTTAACTTCAATGGACTGACCGGAACCGGCGTGCTGACCGAAAGCAACAAGAAAT
TCCTGCCTTTTCAGCAGTTTGGCAGAGACATCGCCGACACCACCGACGCCGTGAGAG
ATCCACAAACCCTGGAAATCCTGGACATCACACCTTGCTCATTTGGAGGGGTGTCGG
TGATCACACCTGGCACCAACACCAGCAACCAGGTGGCCGTGCTGTACCAGGGAGTG
AATTGTACCGAGGTCCCCGTGGCCATTCACGCCGACCAGCTGACCCCTACCTGGCGG
GTGTACTCCACCGGCTCTAACGTATTCCAGACCAGAGCCGGCTGTCTGATCGGCGCA
GAACACGTGAACAATAGCTACGAGTGCGACATCCCTATCGGAGCCGGGATCTGCGCT
AGCTACCAGACCCAGACAAACTCCAGAAGCAGAGCCGGAAGCGTGGCCAGCCAGTC
TATCATCGCCTACACCATGAGCCTGGGCGCCGAAAACAGCGTTGCCTACAGCAACAA
TTCTATCGCCATCCCTACAAACTTCACCATCTCCGTGACCACCGAGATCCTGCCTGTC
AGCATGACAAAGACCAGCGTAGACTGCACAATGTACATCTGCGGAGATTCCACCGA
GTGTAGTAACCTCCTGCTGCAATACGGATCTTTCTGTACTCAGCTGAACAGAGCCCTG
ACCGGCATCGCCGTTGAACAGGACAAGAACACCCAGGAGGTTTTCGCCCAGGTTAA
GCAGATCTACAAAACCCCTCCTATCAAGGACTTCGGAGGCTTTAACTTCTCCCAGAT
CCTGCCCGACCCCAGCAAGCCCAGCAAGCGGAGCCCCATCGAGGACCTGCTGTTCAA
CAAGGTGACCCTGGCCGACGCCGGCTTCATCAAACAGTACGGCGATTGCCTGGGAGA
CATCGCCGCTAGAGATCTAATTTGCGCCCAAAAGTTTAACGGCCTGACAGTGCTGCC
TCCACTGCTGACAGACGAGATGATCGCCCAGTACACATCTGCCCTGCTGGCTGGTAC
CATCACATCTGGCTGGACCTTTGGCGCCGGCCCCGCCCTCCAGATCCCTTTCCCCATG
CAGATGGCCTACCGGTTCAACGGCATCGGCGTGACCCAGAACGTGCTGTACGAAAAC
CAGAAACTGATCGCCAACCAGTTCAATAGCGCGATCGGCAAAATCCAGGATAGCCTC
AGCTCTACACCCAGCGCTCTTGGCAAGCTGCAAAACGTGGTGAACCAGAATGCCCAG
GCCCTTAACACCCTGGTGAAGCAGCTATCCTCTAATTTCGGTGCCATCAGCAGCGTG
CTGAATGATATCCTGAGCAGACTGGACCCCCCTGAGGCCGAAGTGCAGATCGACAG
ACTGATCACCGGAAGACTGCAGAGCCTGCAAACCTACGTGACCCAGCAACTGATCCG
GGCCGCAGAAATCCGGGCCTCCGCTAACCTGGCCGCTACCAAGATGAGCGAGTGCGT
GCTGGGTCAAAGCAAGCGCGTGGACTTCTGTGGAAAAGGCTACCACCTGATGAGCTT
CCCTCAGAGCGCTCCACACGGCGTGGTGTTCCTGCATGTGACTTACGTGCCTGCCCA
GGAAAAGAACTTCACCACCGCCCCTGCCATTTGTCACGACGGCAAGGCCCACTTCCC
CCGGGAAGGCGTGTTTGTGTCTAACGGAACACACTGGTTTGTGACTCAAAGAAACTT
CTACGAGCCACAGATCATCACCACAGATAACACCTTCGTCAGCGGCAACTGCGACGT
GGTGATCGGCATCGTGAACAATACTGTGTACGACCCCCTGCAGCCAGAGCTCGATTC
TTTCAAAGAGGAACTGGATAAGTACTTCAAGAACCACACATCCCCCGACGTCGACCT
GGGCGATATCAGCGGCATTAACGCCAGCGTGGTGAACATCCAGAAGGAAATCGATA
GACTGAACGAGGTGGCAAAGAACCTGAATGAGTCCCTGATTGACCTGCAAGAGCTC
GGGAAGTACGAGCAGTATATCAAGTGGCCTTGGTACATCTGGCTGGGCTTCATCGCG
GGCCTGATCGCCATCGTTATGGTGACGATCATGCTGTGCTGCATGACCAGTTGCTGTA
GCTGCCTGAAGGGCTGCTGCAGCTGCGGCAGCTGTTGCAAGTTCGACGAGGACGACA
GCGAGCCTGTGCTGAAGGGCGTTAAGCTGCACTACACCTGAGCTCGCTTTCTTGCTGT
CCAATTTCTATTAAAGGTTCCTTTGTTCCCTAAGTCCAACTACTAAACTGGGGGATAT
TATGAAGGGCCTTGAGCATCTGGATTCTGCCTAATAAAAAACATTTATTTTCATTGCC
AATAGGCCGAAATCGGCAAGCGCGATCGCAAAAAAAAAAAAAAAAAAAAAAAAAA
AAAAAAAAAAAAAAAAAAAAAAAAGAAAAAAAAAAAAAAAAAAAAAAAAAAAAA
AAAAAAAAAAAAAAAAAAAAAAAAAAAAAAAGAATTCCTCGAGATTTAAATTCGC
GAGTACTATGCATATGGGCCCAATATTAATTAAGCGCTAGCACGCGTTTAAACAGGC
CTCGAGGCGCGCCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAA
AAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGA
GATTATCAAAAAGGATCTTCACCTAGATCCTTTTAAATTAAAAATGAAGTTTTAAATC
AAGCCCAATCTGAATAATGTTACAACCAATTAACCAATTCTGATTAGAAAAACTCAT
CGAGCATCAAATGAAACTGCAATTTATTCATATCAGGATTATCAATACCATATTTTTG
AAAAAGCCGTTTCTGTAATGAAGGAGAAAACTCACCGAGGCAGTTCCATAGGATGG
CAAGATCCTGGTATCGGTCTGCGATTCCGACTCGTCCAACATCAATACAACCTATTA
ATTTCCCCTCGTCAAAAATAAGGTTATCAAGTGAGAAATCACCATGAGTGACGACTG
AATCCGGTGAGAATGGCAAAAGTTTATGCATTTCTTTCCAGACTTGTTCAACAGGCC
AGCCATTACGCTCGTCATCAAAATCACTCGCATCAACCAAACCGTTATTCATTCGTGA
TTGCGCCTGAGCGAGACGAAATACGCGATCGCTGTTAAAAGGACAATTACAAACAG
GAATCGAATGCAACCGGCGCAGGAACACTGCCAGCGCATCAACAATATTTTCACCTG
AATCAGGATATTCTTCTAATACCTGGAATGCTGTTTTTCCGGGGATCGCAGTGGTGAG
TAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACC
TTTGCCATGTTTCAGAAACAACTCTGGCGCATCGGGCTTCCCATACAAGCGATAGAT
TGTCGCACCTGATTGCCCGACATTATCGCGAGCCCATTTATACCCATATAAATCAGCA
TCCATGTTGGAATTTAATCGCGGCCTCGACGTTTCCCGTTGAATATGGCTCATAACAC
CCCTTGTATTACTGTTTATGTAAGCAGACAGTTTTATTGTTCATGATGATATATTTTTA
TCTTGTGCAATGTAACATCAGAGATTTTGAGACACGGGCCAGAGCTGCA
SEQ ID NO: 34, HKP side chain:
KHKHKHKHK
SEQ ID NO: 35, HKP side chain:
HKHKHKHKHK
SEQ ID NO: 36, HKP side chain:
KHKHKHKHKH
SEQ ID NO: 37, HKP side chain:
HKHKHKHKHKH
SEQ ID NO: 38, HKP side chain:
KHKHHKHHKHHKHHKHHKHK
SEQ ID NO: 39, HKP side chain:
KHHHKHHHKHHHKHHHK
SEQ ID NO: 40, HKP side chain:
KHHHKHHHKHHHHKHHHK
SEQ ID NO: 41, HKP side chain:
KHHHKHHHKHHHHKHHHK,
wherein the 1^{st}, 5^{th}, 9^{th}, 14^{th} and 18^{th} amino
acids are D-amino acids.
SEQ ID NO: 42, HKP side chain:
HKHHHKHHHKHHHHKHHHK
SEQ ID NO: 43, HKP side chain:
HHKHHHKHHHKHHHHKHHHK
SEQ ID NO: 44, HKP side chain:
KHHHHKHHHHKHHHHKHHHHK
SEQ ID NO: 45, HKP side chain:
KHHHKHHHKHHHKHHHHK
SEQ ID NO: 46, HKP side chain:
KHHHKHHHHKHHHKHHHK
SEQ ID NO: 47, HKP side chain:
KHHHKHHHHKHHHKHHHHK
SEQ ID NO: 48, furin-like cleavage site:
RRAR
SEQ ID NO: 49:
-HHHK-
RX(K/R)R
SEQ ID NO: 51:
TAATACGACTCACTATAA
SEQ ID NO: 52:
AGGACAUUUGCUUCUGACACAACUGUGUUCACUAGCAACCUCAAACAGACACC
GCCACCAUGUUCGUGUUCCUGGUGCUGCUGCCUCUGGUCAGCAGCCAGUGCGU
GAACCUGAGAACAAGAACACAGCUUCCUCCAGCCUACACAAACUCUUUUACAC
GGGGCGUGUACUAUCCUGACAAGGUGUUCCGGUCCAGCGUGCUGCACUCAACC
CAAGACCUGUUCCUGCCCUUCUUCAGCAACGUCACCUGGUUCCACGCCAUCCA
CGUGUCUGGCACCAAUGGCACAAAGCGAUUCGAUAACCCCGUGCUGCCUUUCA
ACGACGGCGUGUACUUUGCCUCCAUCGAGAAGUCCAACAUCAUCCGGGGCUGG
AUCUUCGGGACCACACUGGAUAGCAAGACCCAGUCUCUGCUGAUCGUAAACAA
CGCCACCAACGUGGUCAUCAAGGUGUGCGAGUUCCAGUUCUGCAACGACCCUU
UCCUCGAUGUGUACUACCACAAGAACAACAAGUCUUGGAUGGAAUCGGGCGU
GUAUAGCAGCGCCAACAACUGCACCUUCGAAUACGUGAGCCAGCCUUUCCUGA
UGGACCUGGAAGGCAAACAAGGCAAUUUUAAGAACCUGAGAGAAUUCGUGUU
CAAAAAUAUAGACGGCUAUUUCAAGAUCUACAGCAAGCACACCCCUAUUAAUC
UGGUGCGGGAUCUGCCUCAGGGCUUCAGCGCCCUCGAACCUCUGGUGGACCUG
CCAAUCGGCAUCAACAUUACAAGAUUCCAGACGCUGCUCGCUCUGCACAGAUC
UUACCUGACCCCUGGCGACAGCAGCAGCGGCUGGACCGCCGGCGCCGCCGCUU
ACUACGUGGGCUACCUGCAGCCUAGAACCUUUCUGCUGAAGUACAACGAGAAC
GGCACCAUCACUGAUGCCGUGGAUUGCGCCCUGGACCCUCUGUCCGAAACCAA
AUGUACACUGAAGUCUUUUACCGUGGAAAAAGGAAUCUACCAGACUUCCAAC
UUCCGGGUGCAGCCGACCGAGAGCAUCGUGCGGUUCCCUAACAUCACAAACCU
GUGCCCCUUUGGCGAGGUGUUCAACGCCACAAGAUUUGCUAGCGUGUACGCCU
GGAAUAGAAAGAGAAUCAGCAACUGCGUGGCCGAUUACAGCGUGCUGUACAA
UAGCGCCUCUUUCAGCACCUUCAAAUGCUACGGCGUGAGCCCCACCAAGCUGA
ACGAUCUGUGUUUUACAAACGUGUAUGCCGACUCAUUCGUAAUCAGGGGCGA
UGAGGUGAGACAGAUCGCUCCUGGACAGACAGGCAAAAUCGCGGACUACAACU
AUAAGCUGCCUGAUGACUUCACAGGAUGUGUGAUCGCAUGGAACUCCAAUAA
CCUCGACAGCAAGGUGGGCGGAAAUUACAAUUACCGCUACAGACUGUUUAGA
AAGAGCAAUCUGAAACCUUUCGAGAGAGACAUCAGCACAGAGAUCUACCAGGC
CGGCAGCAAGCCCUGUAACGGCGUCGAGGGCUUCAACUGCUACUUCCCCCUGC
AGAGCUACGGCUUCCAGCCUACCAACGGCGUGGGAUACCAGCCUUACAGAGUG
GUGGUGCUGAGCUUCGAGCUGCUGCAUGCUCCUGCUACAGUGUGUGGUCCUAA
GAAGAGCACCAACCUGGUUAAGAACAAGUGCGUGAAUUUUAACUUCAAUGGA
CUGACCGGAACCGGCGUGCUGACCGAAAGCAACAAGAAAUUCCUGCCUUUUCA
GCAGUUUGGCAGAGACAUCGCCGACACCACCGACGCCGUGAGAGAUCCACAAA
CCCUGGAAAUCCUGGACAUCACACCUUGCUCAUUUGGAGGGGUGUCGGUGAUC
ACACCUGGCACCAACACCAGCAACCAGGUGGCCGUGCUGUACCAGGGAGUGAA
UUGUACCGAGGUCCCCGUGGCCAUUCACGCCGACCAGCUGACCCCUACCUGGC
GGGUGUACUCCACCGGCUCUAACGUAUUCCAGACCAGAGCAGGCUGUCUGAUC
GGCGCAGAACACGUGAACAAUAGCUACGAGUGCGACAUCCCUAUCGGAGCCGG
GAUCUGCGCUAGCUACCAGACCCAGACAAACUCCAGAAGCAGAGCCGGAAGCG
UGGCCAGCCAGUCUAUCAUCGCCUACACCAUGAGCCUGGGCGCCGAAAACAGC
GUUGCCUACAGCAACAAUUCUAUCGCCAUCCCUACAAACUUCACCAUCUCCGU
GACCACCGAGAUCCUGCCUGUCAGCAUGACAAAGACCAGCGUAGACUGCACAA
UGUACAUCUGCGGAGAUUCCACCGAGUGUAGUAACCUCCUGCUGCAAUACGGA
UCUUUCUGUACUCAGCUGAACAGAGCCCUGACCGGCAUCGCCGUUGAACAGGA
CAAGAACACCCAGGAGGUUUUCGCCCAGGUUAAGCAGAUCUACAAAACCCCUC
CUAUCAAGGACUUCGGAGGCUUUAACUUCUCCCAGAUCCUGCCCGACCCCAGC
AAGCCCAGCAAGCGGAGCCCCAUCGAGGACCUGCUGUUCAACAAGGUGACCCU
GGCCGACGCCGGCUUCAUCAAACAGUACGGCGAUUGCCUGGGAGACAUCGCCG
CUAGAGAUCUAAUUUGCGCCCAAAAGUUUAACGGCCUGACAGUGCUGCCUCCA
CUGCUGACAGACGAGAUGAUCGCCCAGUACACAUCUGCCCUGCUGGCUGGUAC
CAUCACAUCUGGCUGGACCUUUGGCGCCGGCCCCGCCCUCCAGAUCCCUUUCC
CCAUGCAGAUGGCCUACCGGUUCAACGGCAUCGGCGUGACCCAGAACGUGCUG
UACGAAAACCAGAAACUGAUCGCCAACCAGUUCAAUAGCGCGAUCGGCAAAAU
CCAGGAUAGCCUCAGCUCUACACCCAGCGCUCUUGGCAAGCUGCAAAACGUGG
UGAACCAGAAUGCCCAGGCCCUUAACACCCUGGUGAAGCAGCUAUCCUCUAAU
UUCGGUGCCAUCAGCAGCGUGCUGAAUGAUAUCCUGAGCAGACUGGACCCCCC
UGAGGCCGAAGUGCAGAUCGACAGACUGAUCACCGGAAGACUGCAGAGCCUGC
AAACCUACGUGACCCAGCAACUGAUCCGGGCCGCAGAAAUCCGGGCCUCCGCU
AACCUGGCCGCUACCAAGAUGAGCGAGUGCGUGCUGGGUCAAAGCAAGCGCGU
GGACUUCUGUGGAAAAGGCUACCACCUGAUGAGCUUCCCUCAGAGCGCUCCAC
ACGGCGUGGUGUUCCUGCAUGUGACUUACGUGCCUGCCCAGGAAAAGAACUUC
ACCACCGCCCCUGCCAUUUGUCACGACGGCAAGGCCCACUUCCCCCGGGAAGG
CGUGUUUGUGUCUAACGGAACACACUGGUUUGUGACUCAAAGAAACUUCUAC
GAGCCACAGAUCAUCACCACAGAUAACACCUUCGUCAGCGGCAACUGCGACGU
GGUGAUCGGCAUCGUGAACAAUACUGUGUACGACCCCCUGCAGCCAGAGCUCG
AUUCUUUCAAAGAGGAACUGGAUAAGUACUUCAAGAACCACACAUCCCCCGAC
GUCGACCUGGGCGAUAUCAGCGGCAUUAACGCCAGCGUGGUGAACAUCCAGAA
GGAAAUCGAUAGACUGAACGAGGUGGCAAAGAACCUGAAUGAGUCCCUGAUU
GACCUGCAAGAGCUCGGGAAGUACGAGCAGUAUAUCAAGUGGCCUUGGUACA
UCUGGCUGGGCUUCAUCGCGGGCCUGAUCGCCAUCGUUAUGGUGACGAUCAUG
CUGUGCUGCAUGACCAGUUGCUGUAGCUGCCUGAAGGGCUGCUGCAGCUGCGG
CAGCUGUUGCAAGUUCGACGAGGACGACAGCGAGCCUGUGCUGAAGGGCGUU
AAGCUGCACUACACCUGAGCUCGCUUUCUUGCUGUCCAAUUUCUAUUAAAGGU
UCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCCAAUAGG
CCGAAAUCGGCAAGCGCGAUCGCAAAAAAAAAAAAAAAAAAAAAAAAAAAAA
AAAAAAAAAAAAAAAAGAAAAAAAAAAAAAAAAAAAAAAAAAAAAAAAAAAA
AAAAAAAAAAAAAAAAAAAAAAAAGAAUUCC
SEQ ID NO: 53:
ttcctcgaggcgcgcccgcttcctcgctcactgactcgctgcgctcggtcgttcggctgcggc
gagcggtatcagctcactcaaaggcggtaatacggttatccacagaatcaggggataacgcag
gaaagaacatgtgagcaaaaggccagcaaaaggccaggaaccgtaaaaaggccgcgttgctgg
cgtttttccataggctccgcccccctgacgagcatcacaaaaatcgacgctcaagtcagaggt
ggcgaaacccgacaggactataaagataccaggcgtttccccctggaagctccctcgtgcgct
ctcctgttccgaccctgccgcttaccggatacctgtccgcctttctcccttcgggaagcgtgg
cgctttctcatagctcacgctgtaggtatctcagttcggtgtaggtcgttcgctccaagctgg
gctgtgtgcacgaaccccccgttcagcccgaccgctgcgccttatccggtaactatcgtcttg
agtccaacccggtaagacacgacttatcgccactggcagcagccactggtaacaggattagca
gagcgaggtatgtaggcggtgctacagagttcttgaagtggtggcctaactacggctacacta
gaagaacagtatttggtatctgcgctctgctgaagccagttaccttcggaaaaagagttggta
gctcttgatccggcaaacaaaccaccgctggtagcggtggtttttttgtttgcaagcagcaga
ttacgcgcagaaaaaaaggatctcaagaagatcctttgatcttttctacggggtctgacgctc
agtggaacgaaaactcacgttaagggattttggtcattagattatcaaaaaggatcttcacct
agatccttttaaattaaaaatgaagttttaaatcaatctaaagtatatatgagtaaacttggt
ctgacagttagaaaaactcatcgagcatcaaatgaaactgcaatttattcatatcaggattat
caataccatatttttgaaaaagccgtttctgtaatgaaggagaaaactcaccgaggcagttcc
ataggatggcaagatcctggtatcggtctgcgattccgactcgtccaacatcaatacaaccta
ttaatttcccctcgtcaaaaataaggttatcaagtgagaaatcaccatgagtgacgactgaat
ccggtgagaatggcaaaagtttatgcatttctttccagacttgttcaacaggccagccattac
gctcgtcatcaaaatcactcgcatcaaccaaaccgttattcattcgtgattgcgcctgagcga
gacgaaatacgcgatcgctgttaaaaggacaattacaaacaggaatcgaatgcaaccggcgca
ggaacactgccagcgcatcaacaatattttcacctgaatcatgatattcttctaatacctgga
atgctgttttcccagggatcgcagtggtgagtaaccatgcatcatcaggagtacggataaaat
gcttgatggtcggaagaggcataaattccgtcagccagtttagtctgaccatctcatctgtaa
catcattggcaacgctacctttgccatgtttcagaaacaactctggcgcatcgggcttcccat
acaatcgatagattgtcgcacctgattgccctacattatcgcgagcccatttatacccatata
aatcagcatccatgttggaatttaatcgcggcctagagcaagacgtttcccgttgaatatggc
tcatactcttcctttttcaatattattgaagcatttatcagggttattgtctcatgagcggat
acatatttgaatgtatttagaaaaataaacaaataggggttccgcgcacatttccccgaaaag
tgccacctgacgtctaagaaaccattattatcatgacattaacctataaaaataggcgtatca
cgaggccctttcgtctcgcgcgtttcggtgatgacggtgaaaacctctgacacatgcagctcc
cggagacggtcacagcttgtctgtaagcggatgccgggagcagacaagcccgtcagggcgcgt
cagcgggtgttggcgggtgtcggggctggcttaactatgcggcatcagagcagattgtactga
gagtgcaccatatgcggtgtgaaataccgcacagatgcgtaaggagaaaataccgcatcaggc
gccattcgccattcaggctgcgcaactgttgggaagggcgatcggtgcgggcctcttcgctat
tacgccagctggcgaaagggggatgtgctgcaaggcgattaagttgggtaacgccagggtttt
cccagtcacgacgttgtaaaacgacggccagtgaattcgagctcggtacctcgcgaatgcatc
tagatatcggatcccgggcccgtcgactgcagaggcctgcatgcaagctttaatacgactcac
tataaggacatttgcttctgacacaactgtgttcactagcaacctcaaacagacaccgccacc
atgttcgtgttcctggtgctgctgcctctggtcagcagccagtgcgtgaacctgagaacaaga
acacagcttcctccagcctacacaaactcttttacacggggcgtgtactatcctgacaaggtg
ttccggtccagcgtgctgcactcaacccaagacctgttcctgcccttcttcagcaacgtcacc
tggttccacgccatccacgtgtctggcaccaatggcacaaagcgattcgataaccccgtgctg
cctttcaacgacggcgtgtactttgcctccatcgagaagtccaacatcatccggggctggatc
ttcgggaccacactggatagcaagacccagtctctgctgatcgtaaacaacgccaccaacgtg
gtcatcaaggtgtgcgagttccagttctgcaacgaccctttcctcgatgtgtactaccacaag
aacaacaagtcttggatggaatcgggcgtgtatagcagcgccaacaactgcaccttcgaatac
gtgagccagcctttcctgatggacctggaaggcaaacaaggcaattttaagaacctgagagaa
ttcgtgttcaaaaatatagacggctatttcaagatctacagcaagcacacccctattaatctg
gtgcgggatctgcctcagggcttcagcgccctcgaacctctggtggacctgccaatcggcatc
aacattacaagattccagacgctgctcgctctgcacagatcttacctgacccctggcgacagc
agcagcggctggaccgccggcgccgccgcttactacgtgggctacctgcagcctagaaccttt
ctgctgaagtacaacgagaacggcaccatcactgatgccgtggattgcgccctggaccctctg
tccgaaaccaaatgtacactgaagtcttttaccgtggaaaaaggaatctaccagacttccaac
ttccgggtgcagccgaccgagagcatcgtgcggttccctaacatcacaaacctgtgccccttt
ggcgaggtgttcaacgccacaagatttgctagcgtgtacgcctggaatagaaagagaatcagc
aactgcgtggccgattacagcgtgctgtacaatagcgcctctttcagcaccttcaaatgctac
ggcgtgagccccaccaagctgaacgatctgtgttttacaaacgtgtatgccgactcattcgta
atcaggggcgatgaggtgagacagatcgctcctggacagacaggcaaaatcgcggactacaac
tataagctgcctgatgacttcacaggatgtgtgatcgcatggaactccaataacctcgacagc
aaggtgggcggaaattacaattaccgctacagactgtttagaaagagcaatctgaaacctttc
gagagagacatcagcacagagatctaccaggccggcagcaagccctgtaacggcgtcgagggc
ttcaactgctacttccccctgcagagctacggcttccagcctaccaacggcgtgggataccag
ccttacagagtggtggtgctgagcttcgagctgctgcatgctcctgctacagtgtgtggtcct
aagaagagcaccaacctggttaagaacaagtgcgtgaattttaacttcaatggactgaccgga
accggcgtgctgaccgaaagcaacaagaaattcctgccttttcagcagtttggcagagacatc
gccgacaccaccgacgccgtgagagatccacaaaccctggaaatcctggacatcacaccttgc
tcatttggaggggtgtcggtgatcacacctggcaccaacaccagcaaccaggtggccgtgctg
taccagggagtgaattgtaccgaggtccccgtggccattcacgccgaccagctgacccctacc
tggcgggtgtactccaccggctctaacgtattccagaccagagcaggctgtctgatcggcgca
gaacacgtgaacaatagctacgagtgcgacatccctatcggagccgggatctgcgctagctac
cagacccagacaaactccagaagcagagccggaagcgtggccagccagtctatcatcgcctac
accatgagcctgggcgccgaaaacagcgttgcctacagcaacaattctatcgccatccctaca
aacttcaccatctccgtgaccaccgagatcctgcctgtcagcatgacaaagaccagcgtagac
tgcacaatgtacatctgcggagattccaccgagtgtagtaacctcctgctgcaatacggatct
ttctgtactcagctgaacagagccctgaccggcatcgccgttgaacaggacaagaacacccag
gaggttttcgcccaggttaagcagatctacaaaacccctcctatcaaggacttcggaggcttt
aacttctcccagatcctgcccgaccccagcaagcccagcaagcggagccccatcgaggacctg
ctgttcaacaaggtgaccctggccgacgccggcttcatcaaacagtacggcgattgcctggga
gacatcgccgctagagatctaatttgcgcccaaaagtttaacggcctgacagtgctgcctcca
ctgctgacagacgagatgatcgcccagtacacatctgccctgctggctggtaccatcacatct
ggctggacctttggcgccggccccgccctccagatccctttccccatgcagatggcctaccgg
ttcaacggcatcggcgtgacccagaacgtgctgtacgaaaaccagaaactgatcgccaaccag
ttcaatagcgcgatcggcaaaatccaggatagcctcagctctacacccagcgctcttggcaag
ctgcaaaacgtggtgaaccagaatgcccaggcccttaacaccctggtgaagcagctatcctct
aatttcggtgccatcagcagcgtgctgaatgatatcctgagcagactggacccccctgaggcc
gaagtgcagatcgacagactgatcaccggaagactgcagagcctgcaaacctacgtgacccag
caactgatccgggccgcagaaatccgggcctccgctaacctggccgctaccaagatgagcgag
tgcgtgctgggtcaaagcaagcgcgtggacttctgtggaaaaggctaccacctgatgagcttc
cctcagagcgctccacacggcgtggtgttcctgcatgtgacttacgtgcctgcccaggaaaag
aacttcaccaccgcccctgccatttgtcacgacggcaaggcccacttcccccgggaaggcgtg
tttgtgtctaacggaacacactggtttgtgactcaaagaaacttctacgagccacagatcatc
accacagataacaccttcgtcagcggcaactgcgacgtggtgatcggcatcgtgaacaatact
gtgtacgaccccctgcagccagagctcgattctttcaaagaggaactggataagtacttcaag
aaccacacatcccccgacgtcgacctgggcgatatcagcggcattaacgccagcgtggtgaac
atccagaaggaaatcgatagactgaacgaggtggcaaagaacctgaatgagtccctgattgac
ctgcaagagctcgggaagtacgagcagtatatcaagtggccttggtacatctggctgggcttc
atcgcgggcctgatcgccatcgttatggtgacgatcatgctgtgctgcatgaccagttgctgt
agctgcctgaagggctgctgcagctgcggcagctgttgcaagttcgacgaggacgacagcgag
cctgtgctgaagggcgttaagctgcactacacctgagctcgctttcttgctgtccaatttcta
ttaaaggttcctttgttccctaagtccaactactaaactgggggatattatgaagggccttga
gcatctggattctgcctaataaaaaacatttattttcattgccaataggccgaaatcggcaag
cgcgatcgcaaaaaaaaaaaaaaaaaaaaaaaaaaaaaaaaaaaaaaaagaaaaaaaaaaaaa
aaaaaaaaaaaaaaaaaaaaaaaaaaaaaaaaaaaaaaaaaaaaaaa
SEQ ID NO: 54:
Atgtttgtttttcttgttttattgccactagtctctagtcagtgtgttaattttacaaacaga
actcaattaccctctgcatacactaattctttcacacgtggtgtttattaccctgacaaagtt
ttcagatcctcagttttacattcaactcaggacttgttcttacctttcttttccaatgttact
tggttccatgctatacatgtctctgggaccaatggtactaagaggtttgataaccctgtccta
ccatttaatgatggtgtttattttgcttccactgagaagtctaacataataagaggctggatt
tttggtactactttagattcgaagacccagtccctacttattgttaataacgctactaatgtt
gttattaaagtctgtgaatttcaattttgtaattatccatttttgggtgtttattaccacaaa
aacaacaaaagttggatggaaagtgagttcagagtttattctagtgcgaataattgcactttt
gaatatgtctctcagccttttcttatggaccttgaaggaaaacagggtaatttcaaaaatctt
agtgaatttgtgtttaagaatattgatggttattttaaaatatattctaagcacacgcctatt
aatttagtgcgtgatctccctcagggtttttcggctttagaaccattggtagatttgccaata
ggtattaacatcactaggtttcaaactttacttgctttacatagaagttatttgactcctggt
gattcttcttcaggttggacagctggtgctgcagcttattatgtgggttatcttcaacctagg
acttttctattaaaatataatgaaaatggaaccattacagatgctgtagactgtgcacttgac
cctctctcagaaacaaagtgtacgttgaaatccttcactgtagaaaaaggaatctatcaaact
tctaactttagagtccaaccaacagaatctattgttagatttcctaatattacaaacttgtgc
ccttttggtgaagtttttaacgccaccagatttgcatctgtttatgcttggaacaggaagaga
atcagcaactgtgttgctgattattctgtcctatataattccgcatcattttccacttttaag
tgttatggagtgtctcctactaaattaaatgatctctgctttactaatgtctatgcagattca
tttgtaattagaggtgatgaagtcagacaaatcgctccagggcaaactggaaacattgctgat
tataattataaattaccagatgattttacaggctgcgttatagcttggaattctaacaatctt
gattctaaggttggtggtaattataattacctgtatagattgtttaggaagtctaatctcaaa
ccttttgagagagatatttcaactgaaatctatcaggccggtagcacaccttgtaatggtgtt
aaaggttttaattgttactttcctttacaatcatatggtttccaacccacttatggtgttggt
taccaaccatacagagtagtagtactttcttttgaacttctacatgcaccagcaactgtttgt
ggacctaaaaagtctactaatttggttaaaaacaaatgtgtcaatttcaacttcaatggttta
acaggcacaggtgttcttactgagtctaacaaaaagtttctgcctttccaacaatttggcaga
gacattgctgacactactgatgctgtccgtgatccacagacacttgagattcttgacattaca
ccatgttcttttggtggtgtcagtgttataacaccaggaacaaatacttctaaccaggttgct
gttctttatcagggtgttaactgcacagaagtccctgttgctattcatgcagatcaacttact
cctacttggcgtgtttattctacaggttctaatgtttttcaaacacgtgcaggctgtttaata
ggggctgaacatgtcaacaactcatatgagtgtgacatacccattggtgcaggtatatgcgct
agttatcagactcagactaattctccttcgcgggcaggtagtgtagctagtcaatccatcatt
gcctacactatgtcacttggtgcagaaaattcagttgcttactctaataactctattgccata
cccacaaattttactattagtgttaccacagaaattctaccagtgtctatgaccaagacatca
gtagattgtacaatgtacatttgtggtgattcaactgaatgcagcaatcttttgttgcaatat
ggcagtttttgtacacaattaaaccgtgctttaactggaatagctgttgaacaagacaaaaac
acccaagaagtttttgcacaagtcaaacaaatttacaaaacaccaccaattaaagattttggt
ggttttaatttttcacaaatattaccagatccatcaaaaccaagcaagaggtcacctattgaa
gatctacttttcaacaaagtgacacttgcagatgctggcttcatcaaacaatatggtgattgc
cttggtgatattgctgctagagacctcatttgtgcacaaaagtttaacggccttactgttttg
ccacctttgctcacagatgaaatgattgctcaatacacttctgcactgttagcgggtacaatc
acttctggttggacctttggtgcaggtcctgcattacaaataccatttcctatgcaaatggct
tataggtttaatggtattggagttacacagaatgttctctatgagaaccaaaaattgattgcc
aaccaatttaatagtgctattggcaaaattcaagactcactttcttccacaccaagtgcactt
ggaaaacttcaagatgtggtcaaccaaaatgcacaagctttaaacacgcttgttaaacaactt
agctccaattttggtgcaatttcaagtgttttaaatgatatcctttcacgtcttgacccacct
gaggctgaagtgcaaattgataggttgatcacaggcagacttcaaagtttgcagacatatgtg
actcaacaattaattagagctgcagaaatcagagcttctgctaatcttgctgctactaaaatg
tcagagtgtgtacttggacaatcaaaaagagttgatttttgtggaaagggctatcatcttatg
tccttccctcagtcagcacctcatggtgtagtcttcttgcatgtgacttatgtccctgcacaa
gaaaagaacttcacaactgctcctgccatttgtcatgatggaaaagcacactttcctcgtgaa
ggtgtctttgtttcaaatggcacacactggtttgtaacacaaaggaatttttatgaaccacaa
atcattactacagacaacacatttgtgtctggtaactgtgatgttgtaataggaattgtcaac
aacacagtttatgatcctttgcaacctgaattagactcattcaaggaggagttagataaatat
tttaagaatcatacatcaccagatgttgatttaggtgacatctctggcattaatgcttcagtt
gtaaacattcaaaaagaaattgaccgcctcaatgaggttgccaagaatttaaatgaatctctc
atcgatctccaagaacttggaaagtatgagcagtatataaaatggccatggtacatttggcta
ggttttatagctggcttgattgccatagtaatggtgacaattatgctttgctgtatgaccagt
tgctgtagttgtctcaagggctgttgttcttgtggatcctgctgcaaatttgatgaagacgac
tctgagccagtgctcaaaggagtcaaattacattacacataa
SEQ ID NO: 55:
auguucguguuccuggugcugcugccucuggucagcagccagugcgugaaccugagaacaaga
acacagcuuccuccagccuacacaaacucuuuuacacggggcguguacuauccugacaaggug
uuccgguccagcgugcugcacucaacccaagaccuguuccugcccuucuucagcaacgucacc
ugguuccacgccauccacgugucuggcaccaauggcacaaagcgauucgauaaccccgugcug
ccuuucaacgacggcguguacuuugccuccaucgagaaguccaacaucauccggggcuggauc
uucgggaccacacuggauagcaagacccagucucugcugaucguaaacaacgccaccaacgug
gucaucaaggugugcgaguuccaguucugcaacgacccuuuccucgauguguacuaccacaag
aacaacaagucuuggauggaaucgggcguguauagcagcgccaacaacugcaccuucgaauac
gugagccagccuuuccugauggaccuggaaggcaaacaaggcaauuuuaagaaccugagagaa
uucguguucaaaaauauagacggcuauuucaagaucuacagcaagcacaccccuauuaaucug
gugcgggaucugccucagggcuucagcgcccucgaaccucugguggaccugccaaucggcauc
aacauuacaagauuccagacgcugcucgcucugcacagaucuuaccugaccccuggcgacagc
agcagcggcuggaccgccggcgccgccgcuuacuacgugggcuaccugcagccuagaaccuuu
cugcugaaguacaacgagaacggcaccaucacugaugccguggauuggcccuggacccucugu
ccgaaaccaaauguacacugaagucuuuuaccguggaaaaaggaaucuaccagacuuccaacu
uccgggugcagccgaccgagagcaucgugcgguucccuaacaucacaaaccugugccccuuug
gcgagguguucaacgccacaagauuugcuagcguguacgccuggaauagaaagagaaucagca
acugcguggccgauuacagcgugcuguacaauagcgccucuuucagcaccuucaaaugcuacg
gcgugagccccaccaagcugaacgaucuguguuuuacaaacguguaugccgacucauucguaa
ucaggggcgaugaggugagacagaucgcuccuggacagacaggcaaaaucgcggacuacaacu
auaagcugccugaugacuucacaggaugugugaucgcauggaacuccaauaaccucgacagca
aggugggggaaauuacaauuaccgcuacagacuguuuagaaagagcaaucugaaaccuuucga
gagagacaucagcacagagaucuaccaggccggcagcaagcccuguaacggcgucgagggcuu
caacugcuacuucccccugcagagcuacggcuuccagccuaccaacggcgugggauaccagcc
uuacagagugguggugcugagcuucgagcugcugcaugcuccugcuacagugugugguccuaa
gaagagcaccaaccugguuaagaacaagugcgugaauuuuaacuucaauggacugaccggaac
cggcgugcugaccgaaagcaacaagaaauuccugccuuuucagcaguuuggcagagacaucgc
cgacaccaccgacgccgugagagauccacaaacccuggaaauccuggacaucacaccuugcuc
auuuggaggggugucggugaucacaccuggcaccaacaccagcaaccagguggccgugcugua
ccagggagugaauuguaccgagguccccguggccauucacgccgaccagcugaccccuaccug
gggguguacuccaccggcucuaacguauuccagaccagagcaggcugucugaucggcgcagaa
cacgugaacaauagcuacgagugcgacaucccuaucggagccgggaucugcgcuagcuaccag
acccagacaaacuccagaagcagagccggaagcguggccagccagucuaucaucgccuacacc
augagccugggcgccgaaaacagcguugccuacagcaacaauucuaucgccaucccuacaaac
uucaccaucuccgugaccaccgagauccugccugucagcaugacaaagaccagcguagacugc
acaauguacaucugcggagauuccaccgaguguaguaaccuccugcugcaauacggaucuuuc
uguacucagcugaacagagcccugaccggcaucgccguugaacaggacaagaacacccaggag
guuuucgcccagguuaagcagaucuacaaaaccccuccuaucaaggacuucggaggcuuuaac
uucucccagauccugcccgaccccagcaagcccagcaagcggagccccaucgaggaccugcug
uucaacaaggugacccuggccgacgccggcuucaucaaacaguacggcgauugccugggagac
aucgccgcuagagaucuaauuugcgcccaaaaguuuaacggccugacagugcugccuccacug
cugacagacgagaugaucgcccaguacacaucugcccugcuggcugguaccaucacaucuggc
uggaccuuuggcgccggccccgcccuccagaucccuuuccccaugcagauggccuaccgguuc
aacggcaucggcgugacccagaacgugcuguacgaaaaccagaaacugaucgccaaccaguuc
aauagcgcgaucggcaaaauccaggauagccucagcucuacacccagcgcucuuggcaagcug
caaaacguggugaaccagaaugcccaggcccuuaacacccuggugaagcagcuauccucuaau
uucggugccaucagcagcgugcugaaugauauccugagcagacuggaccccccugaggccgaa
gugcagaucgacagacugaucaccggaagacugcagagccugcaaaccuacgugacccagcaa
cugauccgggccgcagaaauccgggccuccgcuaaccuggccgcuaccaagaugagcgagugc
gugcugggucaaagcaagcgcguggacuucuguggaaaaggcuaccaccugaugagcuucccu
cagagcgcuccacacggcgugguguuccugcaugugacuuacgugccugcccaggaaaagaac
uucaccaccgccccugccauuugucacgacggcaaggcccacuucccccgggaaggcguguuu
gugucuaacggaacacacugguuugugacucaaagaaacuucuacgagccacagaucaucacc
acagauaacaccuucgucagcggcaacugcgacguggugaucggcaucgugaacaauacugug
uacgacccccugcagccagagcucgauucuuucaaagaggaacuggauaaguacuucaagaac
cacacaucccccgacgucgaccugggcgauaucagcggcauuaacgccagcguggugaacauc
cagaaggaaaucgauagacugaacgagguggcaaagaaccugaaugagucccugauugaccug
caagagcucgggaaguacgagcaguauaucaaguggccuugguacaucuggcugggcuucauc
ggggccugaucgccaucguuauggugacgaucaugcugugcugcaugaccaguugcuguagcu
gccugaagggcugcugcagcugggcagcuguugcaaguucgacgaggacgacagcgagccugu
gcugaagggcguuaagcugcacuacaccuga

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. A ribonucleic acid (RNA) encoding a spike (S) protein or an immunogenic fragment thereof of a severe acute respiratory syndrome coronavirus 2 (SARS-COV-2) comprising at least one non-naturally occurring amino acid mutation, wherein the at least one non-naturally occurring amino acid mutation comprises one or more of:
a serine (S) as the amino acid corresponding to R682 of SEQ ID NO: 1 (R682S),
a glycine (G) as the amino acid corresponding to R685 of SEQ ID NO: 1 (R685G),
a proline (P) as the amino acid corresponding to F817 of SEQ ID NO: 1 (F817P),
a P as the amino acid corresponding to A892 of SEQ ID NO: 1 (A892P),
a P as the amino acid corresponding to A899 of SEQ ID NO: 1 (A899P),
a P as the amino acid corresponding to A942 of SEQ ID NO: 1 (A942P),
a P as the amino acid corresponding to K986 of SEQ ID NO: 1 (K986P), or
a P as the amino acid corresponding to V987 of SEQ ID NO: 1 (V987P),
with the proviso that the RNA does not comprise SEQ ID NO: 2.

2. The RNA of claim 1, wherein the S protein further comprises one or more of:
a lysine (K) as the amino acid corresponding to N440 of SEQ ID NO: 1 (N440K),
a K as the amino acid corresponding to E484 of SEQ ID NO: 1 (E484K)
an arginine (R) as the amino acid corresponding to T19 of SEQ ID NO: 1 (T19R),
a phenylalanine (F) as the amino acid corresponding to V70 of SEQ ID NO: 1 (V70F),
an isoleucine (I) as the amino acid corresponding to T95 of SEQ ID NO: 1 (T95I),
an aspartic acid (D) as the amino acid corresponding to G142 of SEQ ID NO: 1 (G142D),
a deletion corresponding to E156 of SEQ ID NO: 1 (E156Δ),
a deletion corresponding to F157 of SEQ ID NO: 1 (F157Δ),
a G as the amino acid corresponding to R158 of SEQ ID NO:1 (R158G),
a valine (V) as the amino acid corresponding to A222 of SEQ ID NO: 1 (A222V),
a leucine (L) as the amino acid corresponding to W258 of SEQ ID NO: 1 (W258L),
an asparagine (N) as the amino acid corresponding to K417 of SEQ ID NO: 1 (K417N),
an R as the amino acid corresponding to K417 of SEQ ID NO: 1 (L452R),
a K as the amino acid corresponding to T478 of SEQ ID NO: 1 (T478K),
a G as the amino acid corresponding to D614 of SEQ ID NO: 1 (D614G),
an R as the amino acid corresponding to P681 of SEQ ID NO: 1 (P681R), or
an N as the amino acid corresponding to D950 of SEQ ID NO: 1 (D950N).

3. The RNA of claim 1, wherein the at least one non-naturally occurring amino acid mutation comprises R682S and R685G, optionally wherein the at least one non-naturally occurring amino acid mutation further comprises any one, or any two, or any three, or any four, or any five of F817P, A892P, A899P, A942P, K986P, or V987P, and optionally wherein the at least one non-naturally occurring amino acid mutation comprises or further comprises F817P, A892P, A899P, A942P, K986P, and V987P.

4. The RNA of claim 1, wherein the S protein comprises
the polypeptide of SEQ ID NO: 5 or an equivalent thereof, wherein the equivalent of SEQ ID NO: 5 retains the mutations of T19R, T95I, G142D, E156Δ, F157Δ, R158G, L452R, T478K, D614G, P681R, R682S, R685G, D950N, K986P, and V987P,
the polypeptide of SEQ ID NO: 6 or an equivalent thereof, wherein the equivalent of SEQ ID NO: 6 retains the mutations of T19R, T95I, G142D, E156Δ, F157Δ, R158G, L452R, T478K, D614G, R682S, R685G, D950N, K986P, and V987P,
the polypeptide of SEQ ID NO: 7 or an equivalent thereof, wherein the equivalent of SEQ ID NO: 7 retains the mutations of T19R, T95I, G142D, E156Δ, F157Δ, R158G, L452R, T478K, D614G, P681R, D950N, F817P, A892P, A899P, A942P, K986P, V987P, R682S and R685G,
the polypeptide of SEQ ID NO: 10 or an equivalent thereof, wherein the equivalent of SEQ ID NO: 10 retains the mutations of T19R, T95I, G142D, E156Δ, F157Δ, R158G, L452R, T478K, D614G, D950N, F817P, A892P, A899P, A942P, K986P, V987P, R682S and R685G,
the polypeptide of SEQ ID NO: 11 or an equivalent thereof, wherein the equivalent of SEQ ID NO: 11 retains the mutations of T19R, V70F, T95I, G142D, E156Δ, F157Δ, R158G, A222V, W258L, K417N, L452R, T478K, D614G, P681R, D950N, F817P, A892P, A899P, A942P, K986P, V987P, R682S and R685G, or
the polypeptide of SEQ ID NO: 14 or an equivalent thereof, wherein the equivalent of SEQ ID NO: 14 retains the mutations of T19R, V70F, T95I, G142D, E156Δ, F157Δ, R158G, A222V, W258L, K417N, L452R, T478K, D614G, P681R, D950N, E484K, N440K, F817P, A892P, A899P, A942P, K986P, V987P, R682S and R685G.

5. The RNA of claim 4, comprising
a polynucleotide encoding SEQ ID NO: 5 or an equivalent thereof,
a polynucleotide of encoding SEQ ID NO: 6 or an equivalent thereof,
the polynucleotide of SEQ ID NO: 9 or an equivalent thereof, wherein the equivalent of SEQ ID NO: 9 encodes SEQ ID NO: 7 or an equivalent thereof,
a polynucleotide encoding SEQ ID NO: 10 or an equivalent thereof,
the polynucleotide of SEQ ID NO: 13 or an equivalent thereof, wherein the equivalent of SEQ ID NO: 13 encodes SEQ ID NO: 11 or an equivalent thereof, or
the polynucleotide of SEQ ID NO: 16 or an equivalent thereof, wherein the equivalent of SEQ ID NO: 16 encodes SEQ ID NO: 14 or an equivalent thereof.

6. The RNA of claim 5, wherein the equivalent of any one of SEQ ID NOs: 9, 13, or 16 consists of an GC content of about 35% to about 70% across the full length of the equivalent.

7. The RNA of claim 4, the equivalent is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or more identical to the full-length reference sequence.

8. The RNA of claim 1, further comprising
a 3′ UTR, optionally wherein the 3′UTR comprises any one of SEQ ID NOs: 18, 22, or 24;
a 5′ UTR, optionally wherein the 5′ UTR comprises SEQ ID NO: 20 or 26; or
a polyA tail, optionally wherein the polyA tail comprises any one of SEQ ID NOs: 27, 28, or 30.

9. The RNA of claim 1, comprising or consisting of SEQ ID NO: 32.

10. The RNA of claim 1, wherein the RNA is chemically modified and optionally comprises one or more of: an N1-methyl-pseudouridine residue or a pseudouridine residue.

11. The RNA of claim 10, wherein at least about 50%, or at least about 70%, or about 100% of the uridine residues in the RNA are N1-methyl pseudouridine or pseudouridine.

12. A polynucleotide encoding the RNA of claim 1, or a polynucleotide complementary thereto, optionally wherein the polynucleotide is selected from the group of: a deoxyribonucleic acid (DNA), an RNA, a hybrid of DNA and RNA, or an analog of each thereof.

13. A polynucleotide encoding a spike (S) protein or an immunogenic fragment thereof of a severe acute respiratory syndrome coronavirus 2 (SARS-COV-2) comprising at least one non-naturally occurring amino acid mutation, wherein the polynucleotide comprises the sequence of SEQ ID NO: 55 or an equivalent thereof.

14. A polynucleotide encoding a spike (S) protein or an immunogenic fragment thereof of a severe acute respiratory syndrome coronavirus 2 (SARS-COV-2) comprising at least one non-naturally occurring amino acid mutation, wherein the polynucleotide comprises the sequence of SEQ ID NO: 52 or an equivalent thereof.

15. A vector or cell comprising the polynucleotide of claim 12.

16. A composition comprising a carrier and the RNA of claim 1.

17. The composition of claim 16, wherein the carrier is a pharmaceutically acceptable carrier.

18. A method of producing the RNA of claim 1, comprising culturing the cell of claim 15 under conditions suitable for expressing the RNA.

19. A method of producing the RNA of claim 1, comprising contacting the polynucleotide of claim 12 with an RNA polymerase, adenosine triphosphate (ATP), cytidine triphosphate (CTP), guanosine-5′-triphosphate (GTP), and uridine triphosphate (UTP) or a chemically modified UTP under conditions suitable for expressing the RNA.

20. A composition comprising the RNA of claim 1 and a pharmaceutically acceptable carrier.

21. The composition of claim 20, wherein the pharmaceutically acceptable carrier comprises a lipid nanoparticle (LNP).

22. The composition of claim 21, wherein the LNP comprises a cationic or an ionizable lipid, a cholesterol, a pegylated lipid, and a helper lipid.

23. The composition of claim 20, wherein the pharmaceutically acceptable carrier comprises a polymeric nanoparticle that comprises a Histidine-Lysine co-polymer (HKP), optionally
wherein the HKP comprises a side chain selected from SEQ ID NOs: 34-47, and optionally
wherein the pharmaceutically acceptable carrier further comprises a lipid, optionally a cationic lipid, optionally wherein the cationic lipid is ionizable, or further optionally wherein the cationic lipid comprises Dlin-MC3-DMA (MC3) or dioleoyloxy-3-(trimethylammonio)propane (DOTAP) or both.

24. The composition of claim 23, wherein the lipid further comprises one or more of: a helper lipid, a cholesterol, or a PEGylated lipid, and optionally wherein the pharmaceutically acceptable carrier comprises a lipid nanoparticle (LNP) and optionally wherein the LNP comprises one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof, and optionally wherein the LNP further comprises one or more of: a helper lipid, a cholesterol, or a PEGylated lipid, and optionally wherein the helper lipid comprises one or more of: disteroylphosphatidyl choline (DSPC), Dipalmitoylphosphatidylcholine (DPPC), (2R)-3-(Hexadecanoyloxy)-2-{[(9Z)-octadec-9-enoyl]oxy}propyl 2-(trimethylazaniumyl)ethyl phosphate (POPC), or dioleoyl phosphatidylethanolamine (DOPE), and optionally wherein the cholesterol comprises a plant cholesterol or an animal cholesterol or both.

25. The composition of claim 24, wherein the PEGylated lipid comprises one or more of: PEG-c-DOMG (R-3-[(ω-methoxy-poly(ethyleneglycol)2000)carbamoyl)]-1,2-dimyristyloxypropyl-3-amine), PEG-DSG (1,2-Distearoyl-sn-glycerol, methoxypolyethylene glycol), PEG-DMG (1,2-Dimyristoyl-sn-glycerol) optionally PEG2000-DMG ((1,2-dimyristoyl-sn-glycero-3-phophoethanolamine-N-[methoxy(polyethylene glycol)-2000)], or PEG-DPG (1,2-Dipalmitoyl-sn-glycerol, methoxypolyethylene glycol), and optionally wherein the LNP comprises SM-102, DSPC, cholesterol and PEG2000-DMG, and further optionally wherein the mass ratio of the SM-102, DSPC, cholesterol and PEG200-DMG is about 1:1:1:1 and/or wherein the molar ratio of the SM-102, DSPC, cholesterol and PEG2000-DMG is about 50:10:38.5:1.5.

26. A method of producing the composition of claim 20, comprising contacting the RNA of claim 1 with an HKP, thereby the RNA and the HKP are self-assembled into nanoparticles, and optionally wherein the mass ratio of HKP and the RNA in the contacting step is about 10:1 to about 1:10, optionally 2.5:1, and optionally further comprising contacting the HKP and RNA with cationic lipid, optionally wherein the cationic lipid comprises Dlin-MC3-DMA (MC3) or DOTAP (dioleoyloxy-3-(trimethylammonio)propane) or both, and further optionally wherein the mass ratio of the cationic lipid and the RNA in the contacting step is about 10:1 to about 1:10, optionally 1:1, and optionally wherein the mass ratio of the HKP, the mRNA and the cationic lipid in the contacting step is about 4:1:1.

27. A method of producing a composition, comprising contacting the RNA of claim 1 with a lipid, thereby the RNA and the lipid are self-assembled into lipid nanoparticles (LNPs), and optionally
wherein the LNPs comprise one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof,
and further optionally wherein the LNPs further comprise one or more of: a helper lipid, a cholesterol, or a PEGylated lipid, optionally wherein the helper lipid comprises one or more of: disteroylphosphatidyl choline (DSPC), Dipalmitoylphosphatidylcholine (DPPC), (2R)-3-(Hexadecanoyloxy)-2-{[(9Z)-octadec-9-enoyl]oxy}propyl 2-(trimethylazaniumyl)ethyl phosphate (POPC), or dioleoyl phosphatidylethanolamine (DOPE), optionally wherein the cholesterol comprises a plant cholesterol or an animal cholesterol or both, and optionally wherein the PEGylated lipid comprises one or more of: PEG-c-DOMG (R-3-[(ω-methoxy-poly(ethyleneglycol)2000)carbamoyl)]-1,2-dimyristyloxypropyl-3-amine), PEG-DSG (1,2-Distearoyl-sn-glycerol, methoxypolyethylene glycol), PEG-DMG (1,2-Dimyristoyl-sn-glycerol) optionally PEG2000-DMG ((1,2-dimyristoyl-sn-glycero-3-phophoethanolamine-N-[methoxy(polyethylene glycol)-2000)], or PEG-DPG (1,2-Dipalmitoyl-sn-glycerol, methoxypolyethylene glycol), and optionally wherein the LNPs comprise SM-102, DSPC, cholesterol and PEG2000-DMG.

28. The method of claim 27, wherein the LNPs comprise SM-102, DSPC, cholesterol and PEG2000-DMG, optionally wherein the mass ratio of the SM-102, DSPC, cholesterol and PEG200-DMG is about 1:1:1:1 or optionally wherein the molar ratio of the SM-102, DSPC, cholesterol and PEG2000-DMG is about 50:10:38.5:1.5.

29. A method of one or more of:
(a) preventing a subject from having a symptomatic SARS-COV-2 infection,
(b) inducing an immune response to SARS-COV-2 in a subject in need thereof,
(c) reducing the binding of a SARS-COV-2 or an S protein thereof with angiotensin converting enzyme 2 (ACE2) in a subject in need thereof,
(d) treating a subject infected with SARS-COV-2, or
(e) reducing a SARS-COV-2 viral load in a subject in need thereof,
comprising administering to the subject one or more of: the RNA of claim 1.

30. The method of claim 29, wherein the subject does not have a SARS-COV-2 infection when administrated with the RNA or the composition.


SEQUENCE LISTING (57 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, synthetic construct
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctccttcgcgggcaggtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcacctattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtcctgcattacaaataccatttcctatgcaaatggcttataggtttaatggtattggagttacacagaatgttctctatgagaaccaaaaattgattgccaaccaatttaatagtgctattggcaaaattcaagactcactttcttccacaccaagtgcacttggaaaacttcaagatgtggtcaaccaaaatgcacaagctttaaacacgcttgttaaacaacttagctccaattttggtgcaatttcaagtgttttaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - RNA, 3822 nt, synthetic construct
  source 1..3822: mol_type = other RNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctccttcgcgggcaggtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcacctattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtcctgcattacaaataccatttcctatgcaaatggcttataggtttaatggtattggagttacacagaatgttctctatgagaaccaaaaattgattgccaaccaatttaatagtgctattggcaaaattcaagactcactttcttccacaccaagtgcacttggaaaacttcaagatgtggtcaaccaaaatgcacaagctttaaacacgcttgttaaacaacttagctccaattttggtgcaatttcaagtgttttaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 5 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3816 bp, synthetic construct
  source 1..3816: mol_type = other DNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 9 - RNA, 3816 nt, synthetic construct
  source 1..3816: mol_type = other RNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 10 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1271 aa, synthetic construct
MFVFLVLLPLVSSQCVNLRTRTQLPPAYTNSFTRGVYYPDKVFRSSVLHSTQDLFLPFFSNVTWFHAIHFSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - DNA, 3816 bp, synthetic construct
  source 1..3816: mol_type = other DNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacttctctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgtcctcgaacctctggtggacctgccaatcggcatcaacattacaagattccagacgctgctcgctctgcacagatcttacctgacccctggcgacagcagcagcggcctgaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaacatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 13 - RNA, 3816 nt, synthetic construct
  source 1..3816: mol_type = other RNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacttctctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgtcctcgaacctctggtggacctgccaatcggcatcaacattacaagattccagacgctgctcgctctgcacagatcttacctgacccctggcgacagcagcagcggcctgaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaacatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 14 - protein, 1271 aa, synthetic construct
MFVFLVLLPLVSSQCVNLRTRTQLPPAYTNSFTRGVYYPDKVFRSSVLHSTQDLFLPFFSNVTWFHAIHFSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVGGNYNYRYRLFRKSNLKPFERDISTEIYQAGSKPCNGVKGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3816 bp, synthetic construct
  source 1..3816: mol_type = other DNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacttctctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgtcctcgaacctctggtggacctgccaatcggcatcaacattacaagattccagacgctgctcgctctgcacagatcttacctgacccctggcgacagcagcagcggcctgaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaacatcgcggactacaactataagctgcctgatgacttcacaggatgtgtgatcgcatggaactccaataagctcgacagcaaggtgggcggaaattacaattaccgctacagactgtttagaaagagcaatctgaaacctttcgagagagacatcagcacagagatctaccaggccggcagcaagccctgtaacggcgtcaagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 16 - RNA, 3816 nt, synthetic construct
  source 1..3816: mol_type = other RNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacttctctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgtcctcgaacctctggtggacctgccaatcggcatcaacattacaagattccagacgctgctcgctctgcacagatcttacctgacccctggcgacagcagcagcggcctgaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaacatcgcggactacaactataagctgcctgatgacttcacaggatgtgtgatcgcatggaactccaataagctcgacagcaaggtgggcggaaattacaattaccgctacagactgtttagaaagagcaatctgaaacctttcgagagagacatcagcacagagatctaccaggccggcagcaagccctgtaacggcgtcaagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 17 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 18 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 19 - DNA, 50 bp, synthetic construct
  source 1..50: mol_type = other DNA
acatttgcttctgacacaactgtgttcactagcaacctcaaacagacacc

SEQ ID NO: 20 - RNA, 50 nt, synthetic construct
  source 1..50: mol_type = other RNA
acatttgcttctgacacaactgtgttcactagcaacctcaaacagacacc

SEQ ID NO: 21 - DNA, 55 bp, synthetic construct
  source 1..55: mol_type = other DNA
ggcgctcgagcaggttcagaaggagatcaaaaacccccaaggatcaaacgccacc

SEQ ID NO: 22 - RNA, 55 nt, synthetic construct
  source 1..55: mol_type = other RNA
ggcgctcgagcaggttcagaaggagatcaaaaacccccaaggatcaaacgccacc

SEQ ID NO: 23 - DNA, 50 bp, synthetic construct
  source 1..50: mol_type = other DNA
gggcgctcgagcaggttcagaaggagatcaaaaacccccaaggatcaaac

SEQ ID NO: 24 - RNA, 50 nt, synthetic construct
  source 1..50: mol_type = other RNA
gggcgctcgagcaggttcagaaggagatcaaaaacccccaaggatcaaac

SEQ ID NO: 25 - DNA, 55 bp, synthetic construct
  source 1..55: mol_type = other DNA
ggcgcacgagcagggagagaaggagatcaaaaacccccaaggatcaaacgccacc

SEQ ID NO: 26 - RNA, 55 nt, synthetic construct
  source 1..55: mol_type = other RNA
ggcgcacgagcagggagagaaggagatcaaaaacccccaaggatcaaacgccacc

SEQ ID NO: 27 - RNA, 40 nt, synthetic construct
  source 1..40: mol_type = other RNA
aaaaaaaaaaaaaaaaaaaaaaaaaaaaaaaaaaaaaaaa

SEQ ID NO: 28 - RNA, 121 nt, synthetic construct
  source 1..121: mol_type = other RNA
aaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa

SEQ ID NO: 29 - DNA, 48 bp, synthetic construct
  source 1..48: mol_type = other DNA
cggcaataaaaagacagaataaaacgcacggtgttgggtcgtttgttc

SEQ ID NO: 30 - RNA, 48 nt, synthetic construct
  source 1..48: mol_type = other RNA
cggcaataaaaagacagaataaaacgcacggtgttgggtcgtttgttc

SEQ ID NO: 31 - DNA, 4146 bp, synthetic construct
  source 1..4146: mol_type = other DNA
ggacatttgcttctgacacaactgtgttcactagcaacctcaaacagacaccgccaccatgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctgagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaa

SEQ ID NO: 32 - RNA, 4146 nt, synthetic construct
  source 1..4146: mol_type = other RNA
ggacatttgcttctgacacaactgtgttcactagcaacctcaaacagacaccgccaccatgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctgagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaa

SEQ ID NO: 33 - DNA, 6601 bp, synthetic construct
  source 1..6601: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagctttaatacgactcactataaggacatttgcttctgacacaactgtgttcactagcaacctcaaacagacaccgccaccatgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagccggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctgagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaagaattcctcgagatttaaattcgcgagtactatgcatatgggcccaatattaattaagcgctagcacgcgtttaaacaggcctcgaggcgcgcccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 34 - protein, 9 aa, synthetic construct
KHKHKHKHK

SEQ ID NO: 35 - protein, 10 aa, synthetic construct
HKHKHKHKHK

SEQ ID NO: 36 - protein, 10 aa, synthetic construct
KHKHKHKHKH

SEQ ID NO: 37 - protein, 11 aa, synthetic construct
HKHKHKHKHKH

SEQ ID NO: 38 - protein, 20 aa, synthetic construct
KHKHHKHHKHHKHHKHHKHK

SEQ ID NO: 39 - protein, 17 aa, synthetic construct
KHHHKHHHKHHHKHHHK

SEQ ID NO: 40 - protein, 18 aa, synthetic construct
KHHHKHHHKHHHHKHHHK

SEQ ID NO: 41 - protein, 18 aa, synthetic construct
  SITE 1: note = D-lysine
  SITE 5: note = D-lysine
  SITE 9: note = D-lysine
  SITE 14: note = D-lysine
  SITE 18: note = D-lysine
KHHHKHHHKHHHHKHHHK

SEQ ID NO: 42 - protein, 19 aa, synthetic construct
HKHHHKHHHKHHHHKHHHK

SEQ ID NO: 43 - protein, 20 aa, synthetic construct
HHKHHHKHHHKHHHHKHHHK

SEQ ID NO: 44 - protein, 21 aa, synthetic construct
KHHHHKHHHHKHHHHKHHHHK

SEQ ID NO: 45 - protein, 18 aa, synthetic construct
KHHHKHHHKHHHKHHHHK

SEQ ID NO: 46 - protein, 18 aa, synthetic construct
KHHHKHHHHKHHHKHHHK

SEQ ID NO: 47 - protein, 19 aa, synthetic construct
KHHHKHHHHKHHHKHHHHK

SEQ ID NO: 48 - protein, 4 aa, synthetic construct
RRAR

SEQ ID NO: 49 - protein, 4 aa, synthetic construct
HHHK

SEQ ID NO: 50 - skipped

SEQ ID NO: 51 - DNA, 18 bp, synthetic construct
  source 1..18: mol_type = other DNA
taatacgactcactataa

SEQ ID NO: 52 - RNA, 4149 nt, synthetic construct
  source 1..4149: mol_type = other RNA
aggacatttgcttctgacacaactgtgttcactagcaacctcaaacagacaccgccaccatgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagcaggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctgagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaagaattcc

SEQ ID NO: 53 - DNA, 6599 bp, synthetic construct
  source 1..6599: mol_type = other DNA
ttcctcgaggcgcgcccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcattagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcatgatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgccctacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagctttaatacgactcactataaggacatttgcttctgacacaactgtgttcactagcaacctcaaacagacaccgccaccatgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagcaggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctgagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa

SEQ ID NO: 54 - DNA, 3822 bp, synthetic construct
  source 1..3822: mol_type = other DNA
atgtttgtttttcttgttttattgccactagtctctagtcagtgtgttaattttacaaacagaactcaattaccctctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaattatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagtgaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaacattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctccttcgcgggcaggtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcacctattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtcctgcattacaaataccatttcctatgcaaatggcttataggtttaatggtattggagttacacagaatgttctctatgagaaccaaaaattgattgccaaccaatttaatagtgctattggcaaaattcaagactcactttcttccacaccaagtgcacttggaaaacttcaagatgtggtcaaccaaaatgcacaagctttaaacacgcttgttaaacaacttagctccaattttggtgcaatttcaagtgttttaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 55 - RNA, 3816 nt, synthetic construct
  source 1..3816: mol_type = other RNA
atgttcgtgttcctggtgctgctgcctctggtcagcagccagtgcgtgaacctgagaacaagaacacagcttcctccagcctacacaaactcttttacacggggcgtgtactatcctgacaaggtgttccggtccagcgtgctgcactcaacccaagacctgttcctgcccttcttcagcaacgtcacctggttccacgccatccacgtgtctggcaccaatggcacaaagcgattcgataaccccgtgctgcctttcaacgacggcgtgtactttgcctccatcgagaagtccaacatcatccggggctggatcttcgggaccacactggatagcaagacccagtctctgctgatcgtaaacaacgccaccaacgtggtcatcaaggtgtgcgagttccagttctgcaacgaccctttcctcgatgtgtactaccacaagaacaacaagtcttggatggaatcgggcgtgtatagcagcgccaacaactgcaccttcgaatacgtgagccagcctttcctgatggacctggaaggcaaacaaggcaattttaagaacctgagagaattcgtgttcaaaaatatagacggctatttcaagatctacagcaagcacacccctattaatctggtgcgggatctgcctcagggcttcagcgccctcgaacctctggtggacctgccaatcggcatcaacattacaagattccagacgctgctcgctctgcacagatcttacctgacccctggcgacagcagcagcggctggaccgccggcgccgccgcttactacgtgggctacctgcagcctagaacctttctgctgaagtacaacgagaacggcaccatcactgatgccgtggattgcgccctggaccctctgtccgaaaccaaatgtacactgaagtcttttaccgtggaaaaaggaatctaccagacttccaacttccgggtgcagccgaccgagagcatcgtgcggttccctaacatcacaaacctgtgcccctttggcgaggtgttcaacgccacaagatttgctagcgtgtacgcctggaatagaaagagaatcagcaactgcgtggccgattacagcgtgctgtacaatagcgcctctttcagcaccttcaaatgctacggcgtgagccccaccaagctgaacgatctgtgttttacaaacgtgtatgccgactcattcgtaatcaggggcgatgaggtgagacagatcgctcctggacagacaggcaaaatcgcggactacaactataagctgcctgatgacttcacaggatgtgtgatcgcatggaactccaataacctcgacagcaaggtgggcggaaattacaattaccgctacagactgtttagaaagagcaatctgaaacctttcgagagagacatcagcacagagatctaccaggccggcagcaagccctgtaacggcgtcgagggcttcaactgctacttccccctgcagagctacggcttccagcctaccaacggcgtgggataccagccttacagagtggtggtgctgagcttcgagctgctgcatgctcctgctacagtgtgtggtcctaagaagagcaccaacctggttaagaacaagtgcgtgaattttaacttcaatggactgaccggaaccggcgtgctgaccgaaagcaacaagaaattcctgccttttcagcagtttggcagagacatcgccgacaccaccgacgccgtgagagatccacaaaccctggaaatcctggacatcacaccttgctcatttggaggggtgtcggtgatcacacctggcaccaacaccagcaaccaggtggccgtgctgtaccagggagtgaattgtaccgaggtccccgtggccattcacgccgaccagctgacccctacctggcgggtgtactccaccggctctaacgtattccagaccagagcaggctgtctgatcggcgcagaacacgtgaacaatagctacgagtgcgacatccctatcggagccgggatctgcgctagctaccagacccagacaaactccagaagcagagccggaagcgtggccagccagtctatcatcgcctacaccatgagcctgggcgccgaaaacagcgttgcctacagcaacaattctatcgccatccctacaaacttcaccatctccgtgaccaccgagatcctgcctgtcagcatgacaaagaccagcgtagactgcacaatgtacatctgcggagattccaccgagtgtagtaacctcctgctgcaatacggatctttctgtactcagctgaacagagccctgaccggcatcgccgttgaacaggacaagaacacccaggaggttttcgcccaggttaagcagatctacaaaacccctcctatcaaggacttcggaggctttaacttctcccagatcctgcccgaccccagcaagcccagcaagcggagccccatcgaggacctgctgttcaacaaggtgaccctggccgacgccggcttcatcaaacagtacggcgattgcctgggagacatcgccgctagagatctaatttgcgcccaaaagtttaacggcctgacagtgctgcctccactgctgacagacgagatgatcgcccagtacacatctgccctgctggctggtaccatcacatctggctggacctttggcgccggccccgccctccagatccctttccccatgcagatggcctaccggttcaacggcatcggcgtgacccagaacgtgctgtacgaaaaccagaaactgatcgccaaccagttcaatagcgcgatcggcaaaatccaggatagcctcagctctacacccagcgctcttggcaagctgcaaaacgtggtgaaccagaatgcccaggcccttaacaccctggtgaagcagctatcctctaatttcggtgccatcagcagcgtgctgaatgatatcctgagcagactggacccccctgaggccgaagtgcagatcgacagactgatcaccggaagactgcagagcctgcaaacctacgtgacccagcaactgatccgggccgcagaaatccgggcctccgctaacctggccgctaccaagatgagcgagtgcgtgctgggtcaaagcaagcgcgtggacttctgtggaaaaggctaccacctgatgagcttccctcagagcgctccacacggcgtggtgttcctgcatgtgacttacgtgcctgcccaggaaaagaacttcaccaccgcccctgccatttgtcacgacggcaaggcccacttcccccgggaaggcgtgtttgtgtctaacggaacacactggtttgtgactcaaagaaacttctacgagccacagatcatcaccacagataacaccttcgtcagcggcaactgcgacgtggtgatcggcatcgtgaacaatactgtgtacgaccccctgcagccagagctcgattctttcaaagaggaactggataagtacttcaagaaccacacatcccccgacgtcgacctgggcgatatcagcggcattaacgccagcgtggtgaacatccagaaggaaatcgatagactgaacgaggtggcaaagaacctgaatgagtccctgattgacctgcaagagctcgggaagtacgagcagtatatcaagtggccttggtacatctggctgggcttcatcgcgggcctgatcgccatcgttatggtgacgatcatgctgtgctgcatgaccagttgctgtagctgcctgaagggctgctgcagctgcggcagctgttgcaagttcgacgaggacgacagcgagcctgtgctgaagggcgttaagctgcactacacctga

SEQ ID NO: 56 - DNA, 40 bp, synthetic construct
  source 1..40: mol_type = other DNA
aaaaaaaaaaaaaaaaaaaaaaaaaaaaaaaaaaaaaaaa

SEQ ID NO: 57 - DNA, 121 bp, synthetic construct
  source 1..121: mol_type = other DNA
aaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa